SEQUENCE LISTING (57 sequences)

SEQ ID NO: 1 - protein, 5 aa, Mus musculus
TYAMN

SEQ ID NO: 2 - protein, 19 aa, Mus musculus
RIRSKYNNYATYYADSVKG

SEQ ID NO: 3 - protein, 14 aa, Mus musculus
HGNFGNSYVSWFAY

SEQ ID NO: 4 - protein, 14 aa, Mus musculus
GSSTGAVTTSNYAN

SEQ ID NO: 5 - protein, 7 aa, Mus musculus
GTNKRAP

SEQ ID NO: 6 - protein, 9 aa, Mus musculus
ALWYSNLWV

SEQ ID NO: 7 - protein, 125 aa, Mus musculus
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 8 - protein, 109 aa, Mus musculus
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 116 aa, Artificial Sequence
  Variable region VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 10 - protein, 116 aa, Artificial Sequence
  Variable region VH
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSS

SEQ ID NO: 11 - protein, 109 aa, Artificial Sequence
  Variable region VL 83A10
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 12 - protein, 109 aa, Artificial Sequence
  Variable region VL
EIVLTQSPGTLSLSPGERATLSCRASQSVSEYYLAWYQQKPGQAPRLLIEHASTRATGIPDRFSGSGSGTDFTLTISRLEPEDFAVYYCQQYGYPPDFTFGQGTKVEIK

SEQ ID NO: 13 - protein, 109 aa, Artificial Sequence
  Variable region VL Mab22
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIK

SEQ ID NO: 14 - protein, 109 aa, Artificial Sequence
  Variable region VL Mab42
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIK

SEQ ID NO: 15 - protein, 5 aa, Homo sapiens
  83A10 CDR1H
SYAMS

SEQ ID NO: 16 - protein, 17 aa, Homo sapiens
  83A10 CDR2H
AISGSGGSTYYADSVKG

SEQ ID NO: 17 - protein, 7 aa, Homo sapiens
  CDRH3
VLGWFDY

SEQ ID NO: 18 - protein, 13 aa, Homo sapiens
  83A10 CDR1L
RASQSVSSSYLAW

SEQ ID NO: 19 - protein, 8 aa, Homo sapiens
  83A10 CDR2L
YGASSRAT

SEQ ID NO: 20 - protein, 10 aa, Homo sapiens
  CDRL3
QQYGYPPDFT

SEQ ID NO: 21 - protein, 5 aa, Artificial Sequence
  CDR1H
DNAMG

SEQ ID NO: 22 - protein, 17 aa, Artificial Sequence
  CDR2H
AISGPGSSTYYADSVKG

SEQ ID NO: 23 - protein, 13 aa, Artificial Sequence
  Mab21 CDR1L
RASQSVSEYYLAW

SEQ ID NO: 24 - protein, 8 aa, Artificial Sequence
  Mab21 CDR2L
EHASTRAT

SEQ ID NO: 25 - protein, 13 aa, Artificial Sequence
  Mab22 CDR1L
RASQSVSSYYLAW

SEQ ID NO: 26 - protein, 8 aa, Artificial Sequence
  Mab22 CDR2L
SGAGSRAT

SEQ ID NO: 27 - protein, 13 aa, Artificial Sequence
  Mab42 CDR1L
RASQSVSDEYLSW

SEQ ID NO: 28 - protein, 8 aa, Artificial Sequence
  Mab42 CDR2L
HSASTRAT

SEQ ID NO: 29 - protein, 5 aa, Artificial Sequence
  Mab27 CDR1H
SAPMG

SEQ ID NO: 30 - protein, 16 aa, Artificial Sequence
  Mab27 CDR2H
AISYIGHTYYADSVKG

SEQ ID NO: 31 - protein, 12 aa, Artificial Sequence
  CDR1L
RASQSVSEYYLA

SEQ ID NO: 32 - protein, 7 aa, Artificial Sequence
  CDR2L
HASTRAT

SEQ ID NO: 33 - protein, 10 aa, Artificial Sequence
  CDR3L
QQYGYPPDFT

SEQ ID NO: 34 - protein, 5 aa, Artificial Sequence
  Mab33 CDR1H
TNAMG

SEQ ID NO: 35 - protein, 17 aa, Artificial Sequence
  Mab33 CDR2H
AINRFGGSTYYADSVKG

SEQ ID NO: 36 - protein, 5 aa, Artificial Sequence
  Mab39 CDR1H
QNAMG

SEQ ID NO: 37 - protein, 17 aa, Artificial Sequence
  Mab39 CDR2H
AISPTGFSTYYADSVKG

SEQ ID NO: 38 - protein, 115 aa, Artificial Sequence
  Mab27 VH
EVQLLESGGGLVQPGGSLRLSCAASGFTFSSAPMGWVRQAPGKGLEWVSAISYIGHTYYADSVKGRFTISRDNSKNTLYLQMNSLRAEDTAVYYCAKVLGWFDYWGQGTLVTVSS

SEQ ID NO: 39 - protein, 116 aa, Artificial Sequence
  Mab33 VH
EVQLLESGGGLVQPGGSLRLSCAASGFTFYTNAMGWVRQAPGKGLEWVSAINRFGGSTYYADSVKGRFTISRDNSKNTLYLQMNSLRAEDTAVYYCAKVLGWFDYWGQGTLVTVSS

SEQ ID NO: 40 - protein, 116 aa, Artificial Sequence
  Mab39 VH
EVQLLESGGGLVQPGGSLRLSCAASGFTFTQNAMGWVRQAPGKGLEWVSAISPTGFSTYYADSVKGRFTISRDNSKNTLYLQMNSLRAEDTAVYYCAKVLGWFDYWGQGTLVTVSS

SEQ ID NO: 41 - protein, 103 aa, Artificial Sequence
  CH1 domain
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 42 - protein, 107 aa, Artificial Sequence
  CL domain
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 103 aa, Artificial Sequence
  CD3 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 107 aa, Artificial Sequence
  CD3 CL
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 671 aa, Artificial Sequence
  83A10 knob HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 446 aa, Artificial Sequence
  83A10 hole HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 216 aa, Artificial Sequence
  83A10 LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 232 aa, Artificial Sequence
  CD3 LC
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 671 aa, Artificial Sequence
  Mab21 knob HC
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 446 aa, Artificial Sequence
  Mab21 hole HC
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 216 aa, Artificial Sequence
  Mab21 LC
EIVLTQSPGTLSLSPGERATLSCRASQSVSEYYLAWYQQKPGQAPRLLIEHASTRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 671 aa, Artificial Sequence; identical to SEQ ID NO: 49
  Mab22 knob HC

SEQ ID NO: 53 - protein, 446 aa, Artificial Sequence; identical to SEQ ID NO: 50
  Mab22 hole HC

SEQ ID NO: 54 - protein, 216 aa, Artificial Sequence
  Mab22 LC
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 671 aa, Artificial Sequence; identical to SEQ ID NO: 49
  Mab42 knob HC

SEQ ID NO: 56 - protein, 446 aa, Artificial Sequence; identical to SEQ ID NO: 50
  Mab42 hole HC

SEQ ID NO: 57 - protein, 216 aa, Artificial Sequence
  Mab42 LC
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0057602 A1
(43) Pub. Date: Feb. 23, 2023

(54) METHODS OF TREATMENT WITH ANTIBODIES AGAINST BCMA AND CD3

(71) Applicant: BRISTOL-MYERS SQUIBB COMPANY, Princeton, NJ (US)

(72) Inventors: Michael BURGESS, San Francisco, CA (US); Kristen HEGE, Burlingame, CA (US); Kaushik DATTA, New Providence, NJ (US); Isaac BOSS, Seattle, WA (US); Minh Diem VU, Wollerau (CH)

(21) Appl. No.: 17/772,865

(22) PCT Filed: Nov. 4, 2020
(86) PCT No.: PCT/US2020/058939

§ 371 Date: Apr. 28, 2022

(30) Foreign Application Priority Data
Nov. 5, 2019 (EP) 19207293.2
Jun. 11, 2020 (EP) 20179573.9

(51) Int. Cl. C07K 16/28; A61P 35/00

(52) U.S. Cl. CPC C07K 16/2809; A61P 35/00; C07K 16/2878; A61K 2039/505

(57) ABSTRACT

The present invention relates to methods of treating a patient having a disorder associated with BCMA expression (e.g. BCMA-expressing B-cell cancers, such as multiple myeloma) using dose-escalation dosing regimens with multispecific (e.g. bispecific) antibodies that bind to CD3 and BCMA.

### CROSS-REFERENCE TO RELATED APPLICATIONS

This application claims the benefit of European Application No. 19207293.2 filed Nov. 5, 2019 and European Application No. 20179573.9 filed Jun. 11, 2020, the content of each of which is incorporated herein by reference in its entirety.

### REFERENCE TO SEQUENCE LISTING SUBMITTED ELECTRONICALLY

This application incorporates by reference a Sequence Listing submitted with this application as text file entitled “14247-608-228_Sequence_Listing.TXT” created on Nov. 4, 2020 and having a size of68,146 bytes.

### FIELD OF THE INVENTION

The present invention relates to antibodies against BCMA and CD3 for use in the treatment of a disorder associated with BCMA expression (e.g. BCMA-expressing B-cell cancers, such as multiple myeloma).

### BACKGROUND

Multispecific (e.g. bispecific) antibodies against BCMA and CD3 are known, and have demonstrated remarkable therapeutic efficacy. However, these antibodies can be associated with adverse effects, most notably cytokine release syndrome (CRS). Thus, there is a need for a dosing regimen which achieves a favorable benefit-risk profile.

### SUMMARY

The present invention relates to methods of treating a patient having a disorder associated with BCMA expression (e.g. BCMA-expressing B-cell cancers, such as multiple myeloma) using dose-escalation dosing regimens with multispecific (e.g. bispecific) antibodies that bind to CD3 and BCMA. This dosage regimen significantly reduces toxicity due to attenuation of cytokine release.

Thus, in one aspect, the present invention provides a method for treating a disorder associated with BCMA expression (e.g. BCMA-expressing B-cell cancers, such as multiple myeloma) in a patient (e.g. a human), wherein the treatment comprises the administration of a multispecific (e.g. bispecific) antibody that binds to BCMA and CD3 in a dosing regimen comprising:
(i) a starting phase, wherein one or more starting doses of the multispecific (e.g. bispecific) antibody are administered to the patient; and
(ii) a maintenance phase, wherein a first maintenance dose of the multispecific (e.g. bispecific) antibody is administered to the patient, optionally followed by at least one additional maintenance dose of the multispecific (e.g. bispecific) antibody;

wherein each maintenance dose is greater than the one or more starting doses.

In another aspect, the present invention provides a multispecific (e.g. bispecific) antibody that binds to BCMA and CD3 for use in treating a disorder associated with BCMA expression (e.g. BCMA-expressing B-cell cancers, such as multiple myeloma) in a patient (e.g. a human), wherein the treatment comprises the administration of the multispecific (e.g. bispecific) antibody in a dosing regimen which comprises:
(i) a starting phase, wherein one or more starting doses of the multispecific (e.g. bispecific) antibody are administered to the patient; and
(ii) a maintenance phase, wherein a first maintenance dose of the multispecific (e.g. bispecific) antibody is administered to the patient, optionally followed by at least one additional maintenance dose of the multispecific (e.g. bispecific) antibody;

wherein each maintenance dose is greater than the one or more starting doses.

In some embodiments, the one or more starting doses comprise a fixed dose of about 1.5 mg to 4.5 mg; from about 2 mg to 4 mg; from about 2.5 mg to 3.5 mg, e.g. about 3 mg. In preferred embodiments, the one or more starting doses comprise a single fixed dose of about 1.5 mg to 4.5 mg; from about 2 mg to 4 mg; from about 2.5 mg to 3.5 mg, e.g. about 3 mg.

In some embodiments, the first maintenance dose may be administered at a fixed dose of about 4.5 mg to 7.5 mg; from about 5 mg to 6 mg; from about 5.5 mg to 6.5 mg, e.g. about 6 mg.

In some embodiments, the at least one additional maintenance dose is the same as the first maintenance dose. In some embodiments, the maintenance dose may be administered at a fixed dose of about 4.5 mg to 7.5 mg; from about 5 mg to 6 mg; from about 5.5 mg to 6.5 mg, e.g. about 6 mg. Thus, in some embodiments, the starting dose of the multispecific (e.g. bispecific) antibody is a (e.g. single) fixed dose of about 3 mg and each maintenance dose is a fixed dose of about 6 mg.

In some embodiments, the at least one additional maintenance dose is greater than the first maintenance dose. In some embodiments, the first maintenance dose may be administered at a fixed dose of about 4.5 mg to 7.5 mg; from about 5 mg to 6 mg; from about 5.5 mg to 6.5 mg, e.g. about 6 mg, and the at least one additional maintenance dose is a fixed dose of about 8.5 mg to 11.5 mg; from about 9 mg to 11 mg; from about 9.5 mg to 10.5 mg, e.g. about 10 mg. Thus, in some embodiments, the starting dose of the multispecific (e.g. bispecific) antibody is a (e.g. single) fixed dose of about 3 mg, the first maintenance dose is a fixed dose of about 6 mg and the at least one additional maintenance dose is a fixed dose of about 10 mg.

In some embodiments, the one or more starting doses comprise a fixed dose of about 4.5 mg to 7.5 mg; from about 5 mg to 7 mg; from about 6.5 mg to 7.5 mg, e.g. about 6 mg. In some embodiments, the one or more starting doses comprise a single fixed dose of about 4.5 mg to 7.5 mg; from about 5 mg to 7 mg; from about 5.5 mg to 6.5 mg, e.g. about 6 mg.

In some embodiments, the first maintenance dose may be administered at a fixed dose of about 8.5 mg to 11.5 mg; from about 9 mg to 11 mg; from about 9.5 mg to 10.5 mg, e.g. about 10 mg.

In some embodiments, the at least one additional maintenance dose is the same as the first maintenance dose. In some embodiments, the maintenance dose may be administered at a fixed dose of about 8.5 mg to 11.5 mg; from about 9 mg to 11 mg; from about 9.5 mg to 10.5 mg, e.g. about 10 mg. Thus, in some embodiments, the starting dose of the multispecific (e.g. bispecific) antibody is a fixed dose of about 6 mg and each maintenance dose is a fixed dose of about 10 mg.

In some embodiments, the at least one additional maintenance dose is greater than the first maintenance dose. Thus, in some embodiments, the starting dose of the multispecific (e.g. bispecific) antibody is a fixed dose of about 6 mg, the first maintenance dose is a fixed dose of about 10 mg and the at least one additional maintenance dose is a fixed dose greater than about 10 mg.

In some embodiments, the patient has developed, or is at risk of developing, an adverse event associated with the administration of the multispecific (e.g. bispecific) antibody, and wherein the treatment further comprises the administration of:
a) a steroid, e.g. a corticosteroid;
b) an antagonist of a cytokine receptor or cytokine selected from among GM-CSF, IL-10, IL-10R, IL-6, IL-6 receptor (IL-6R), IFNγ, IFNGR, IL-2, IL-2R/CD25, MCP-1, CCR2, CCR4, MIPIβ, CCR5, TNFalpha, TNFR1, IL-1, and IL-1Ralpha/IL-1beta, wherein the antagonist is selected from an antibody or antigen-binding fragment, a small molecule, a protein or peptide and a nucleic acid;
c) a molecule that decreases the regulatory T cell (Treg) population, e.g. cyclophosphamide;
d) an antipyretic, analgesics and/or antibiotics; and/or
e) a seizure prophylaxis, e.g. levetiracetam.

In some embodiments, the corticosteroid is dexamethasone or methylprednisolone. In some embodiments, the antagonist is tocilizumab and/or siltuximab.

In some embodiments, the multispecific (e.g. bispecific) antibody is administered intravenously or subcutaneously. In preferred embodiments, the multispecific (e.g. bispecific) antibody is administered intravenously.

In some embodiments, the disorder associated with BCMA expression is a BCMA-expressing B-cell cancer, such as multiple myeloma. In some embodiments, the multispecific (e.g. bispecific) antibody is administered to the patient as a monotherapy. In some embodiments, the multispecific (e.g. bispecific) antibody is administered to the patient as a combination therapy with one or more additional therapeutic agents. In some embodiments, the one or more additional therapeutic agents are selected from the group consisting of thalidomide and an immunotherapeutic derivative thereof, an anti-CD38 antibody, an anti-PD-1 antibody, an anti-PD-L1 antibody, a gamma secretase inhibitor (GSI), an anti-BCMA antibody drug conjugate and anti-BCMA CAR T-cell therapy.

In certain embodiments, the “subject” or “patient” is a human.

In some embodiments, the multispecific (e.g. bispecific) antibody comprises an anti-BCMA antibody, or antigen binding fragment thereof, comprising a CDR3H region of SEQ ID NO: 17 and a CDR3L region of SEQ ID NO:20 and a CDR1H, CDR2H, CDR1L, and CDR2L region combination selected from the group of:
a) CDR1H region of SEQ ID NO:21 and CDR2H region of SEQ ID NO:22, CDR1L region of SEQ ID NO:23, and CDR2L region of SEQ ID NO:24,
b) CDR1H region of SEQ ID NO:21 and CDR2H region of SEQ ID NO:22, CDR1L region of SEQ ID NO:25, and CDR2L region of SEQ ID NO:26,
c) CDR1H region of SEQ ID NO:21 and CDR2H region of SEQ ID NO:22, CDR1L region of SEQ ID NO:27, and CDR2L region of SEQ ID NO:28,
d) CDR1H region of SEQ ID NO:29 and CDR2H region of SEQ ID NO:30, CDR1L region of SEQ ID NO:31, and CDR2L region of SEQ ID NO:32,
e) CDR1H region of SEQ ID NO:34 and CDR2H region of SEQ ID NO:35, CDR1L region of SEQ ID NO:31, and CDR2L region of SEQ ID NO:32,
f) CDR1H region of SEQ ID NO:36 and CDR2H region of SEQ ID NO:37, CDR1L region of SEQ ID NO:31, and CDR2L region of SEQ ID NO:32, and
g) CDR1H region of SEQ ID NO: 15 and CDR2H region of SEQ ID NO: 16, CDR1L region of SEQ ID NO:18, and CDR2L region of SEQ ID NO:19.

In some embodiments, the anti-BCMA antibody, or antigen binding fragment thereof, comprises a VH and a VL selected from the group consisting of:
a) a VH region of SEQ ID NO: 10 and a VL region of SEQ ID NO: 12,
b) a VH region of SEQ ID NO:10 and a VL region of SEQ ID NO:13,
c) a VH region of SEQ ID NO: 10 and a VL region of SEQ ID NO: 14,
d) a VH region of SEQ ID NO:38 and a VL region of SEQ ID NO: 12,
e) a VH region of SEQ ID NO:39 and a VL region of SEQ ID NO: 12,
f) a VH region of SEQ ID NO:40 and a VL region of SEQ ID NO: 12, or
g) a VH region of SEQ ID NO:9 and a VL region of SEQ ID NO:11.

In particularly preferred embodiments, the anti-BCMA antibody, or antigen binding fragment thereof, comprises a VH region of SEQ ID NO: 10 and a VL region of SEQ ID NO: 14.

In some embodiments, the multispecific (e.g. bispecific) antibody comprises an anti-CD3 antibody, or antigen binding fragment thereof, comprising a variable domain VH comprising the heavy chain CDRs of SEQ ID NO: 1, 2 and 3 as respectively heavy chain CDR1H, CDR2H and CDR3H and a variable domain VL comprising the light chain CDRs of SEQ ID NO: 4, 5 and 6 as respectively light chain CDR1L, CDR2L and CDR3L. In some embodiments, the anti-CD3 antibody, or antigen binding fragment thereof, comprises a VH region of SEQ ID NO:7 and a VL region of SEQ ID NO:8.

In particularly preferred embodiments, the multispecific (e.g. bispecific) antibody comprises an anti-BCMA antibody, or antigen binding fragment thereof, comprising a VH region of SEQ ID NO: 10 and a VL region of SEQ ID NO: 14, and an anti-CD3 antibody, or antigen binding fragment thereof, comprising a VH region of SEQ ID NO:7 and a VL region of SEQ ID NO:8.

In some embodiments, the multispecific antibody is a bispecific antibody. In some embodiments, the bispecific antibody is bivalent (the 1+1 format). In some embodiments, the bivalent bispecific antibody has the format: CD3 Fab-BCMA Fab (i.e. when no Fc is present). Alternatively, the bivalent bispecific antibody may have the format: Fc-CD3 Fab-BCMA Fab; Fc-BCMA Fab-CD3 Fab; or BCMA Fab-Fc-CD3 Fab (i.e. when an Fc is present). In preferred embodiments, the bivalent bispecific antibody has the format BCMA Fab-Fc-CD3 Fab. In some embodiments, the bispecific antibody is trivalent (the 2+1 format). In some embodiments, the trivalent bispecific antibody has the format: CD3 Fab-BCMA Fab-BCMA Fab; or BCMA Fab-CD3 Fab-BCMA Fab (i.e. when no Fc is present). Alternatively, the trivalent bispecific antibodies may have the format: BCMA Fab-Fc-CD3 Fab-BCMA Fab; BCMA Fab-Fc-BCMA Fab-CD3 Fab; or CD3 Fab-Fc-BCMA Fab-BCMA Fab (i.e. when an Fc is present). In preferred embodiments, the trivalent bispecific antibody has the format BCMA Fab-Fc-CD3 Fab-BCMA Fab.

In some embodiments, the anti-CD3 Fab comprises a light chain and a heavy chain, wherein the light chain is a crossover light chain that comprises a variable domain VH and a constant domain CL, and wherein the heavy chain is a crossover heavy chain that comprises a variable domain VL and a constant domain CH1.

In some embodiments, the CH1 domain of the anti-BCMA Fab fragment comprises the amino acid modifications K147E/D and K213E/D (numbered according to EU numbering) and a corresponding immunoglobulin light chain comprising a CL domain having amino acid modifications E123K/R/H and Q124K/R/H (numbered according to Kabat).

In some embodiments, the multispecific (e.g. bispecific) antibody further comprises an Fc. In some embodiments, the Fc is an IgG1 Fc. In some embodiments, the (e.g. IgG1) Fc comprises a first Fc chain comprising first constant domains CH2 and CH3, and a second Fc chain comprising second constant domains CH2 and CH3, and wherein:
a) the first CH3 domain comprises the modifications T366S, L368A and Y407V, or conservative substitutions thereof (numbered according to EU numbering); and
b) the second CH3 domain comprises the modifications T366W, or conservative substitutions thereof (numbered according to EU numbering).

In some embodiments, the (e.g. IgG1) Fc comprises:
a) the modifications L234A, L235A and P329G (numbered according to EU numbering); and/or
b) the modifications D356E, and L358M (numbered according to EU numbering).

In further embodiments, the bispecific antibody according to the invention comprises the following SEQ ID NOs:
i. 83A10-TCBcv: 45, 46, 47 (×2), 48 (FIG. 2A)
ii. 21-TCBcv: 48, 49, 50, 51 (×2) (FIG. 2A)
iii. 22-TCBcv: 48, 52, 53, 54 (×2) (FIG. 2A)
iv. 42-TCBcv: 48, 55, 56, 57 (×2) (FIG. 2A)

In a preferred embodiment, the bispecific antibody according to the invention is 42-TCBcv.

Aspects and embodiments of the invention are set out in the appended claims. These and other aspects and embodiments of the invention are also described herein.

### BRIEF DESCRIPTION OF FIGURES

The present invention will now be described in more detail with reference to the attached Figures, in which:

FIG. 1 illustrates different formats of bispecific bivalent antibodies for use in the present invention, which comprise Fab fragments binding to CD3 and BCMA in the format Fab BCMA-Fc-Fab CD3. The CD3 Fab may include a VH-VL crossover to reduce light chain mispairing and side-products. Amino acid substitutions “RK/EE” may be introduced in CL-CH1 to reduce light chain mispairing/side products in production. The CD3 Fab and BCMA Fab may be linked to each other with flexible linkers.

FIG. 2 illustrates different formats of bispecific trivalent antibodies for use in the present invention, which comprise Fab fragments binding to CD3 and BCMA in the following formats: Fab BCMA-Fc-Fab CD3-Fab BCMA (A,B); Fab BCMA-Fc-Fab BCMA-Fab CD3 (C,D). The CD3 Fab may include a VH-VL crossover to reduce light chain mispairing and side-products. Amino acid substitutions “RK/EE” may be introduced in CL-CH1 to reduce light chain mispairing/side products in production. The CD3 Fab and BCMA Fab may be linked to each other with flexible linkers.

FIG. 3 illustrates different formats of bispecific trivalent antibodies for use in the present invention, which comprise Fab fragments binding to CD3 and BCMA in the following formats: Fc-Fab CD3-Fab BCMA (A, B); Fc-Fab BCMA-Fab CD3 (C, D). The CD3 Fab may include a VH-VL crossover to reduce light chain mispairing and side-products. Amino acid substitutions “RK/EE” may be introduced in CL-CH1 to reduce light chain mispairing/side products in production. The CD3 Fab and BCMA Fab may be linked to each other with flexible linkers

FIG. 4 illustrates Cytokine Release Syndrome events across all subjects in the clinical study of CC-93269 in Relapsed/Refractory Multiple Myeloma (RRMM) of Examples 1 and 2.

FIG. 5 illustrates the frequency of Cytokine Release Syndrome events across all subjects in the clinical study of CC-93269 in Relapsed/Refractory Multiple Myeloma (RRMM) of Examples 1 and 2.

FIG. 6 illustrates the effect of dexamethasone on CC-93269-induced cytokine secretion as described in Example 3. Cytokines (pg/mL) are graphed as mean±standard deviation of triplicate samples. H929, MM1S, KMS12-PE and SKMM2 are BCMA-expressing myeloma cell lines. Dex=dexamethasone.

FIG. 7 illustrates the effect of dexamethasone on CC-93269-induced lysis of BCMA-expressing myeloma cell lines (H929, MM.1S, KMS12-PE and SKMM2) as described in Example 3. Percentages of live tumour cells are graphed as mean±standard deviation of triplicate samples. Dex=dexamethasone.

FIG. 8 illustrates the effect of dexamethasone on CC-93269-induced T cell proliferation and activation as described in Example 3. Proliferation is measured as percentage of CD4⁺/CD8⁺ T-cells that show dilution of CellTrace Violet following coculture with tumor cell lines, compared to T-cell only cultures. Expression of activation markers CD25, CD69 and HLA-DR on CD4⁺ and CD8⁺ T cells is measured as percentage of CD4⁺/CD8⁺ T-cells expressing the activation markers following coculture with tumor cell lines, compared to T-cell only cultures. Both proliferation and expression of activation markers are graphed as mean standard deviation of triplicate samples. SKMM2 is a BCMA-expressing myeloma cell line. Dex=dexamethasone.

### DETAILED DESCRIPTION

As used herein, the articles “a” and “an” may refer to one or to more than one (e.g. to at least one) of the grammatical object of the article.

“About” may generally mean an acceptable degree of error for the quantity measured given the nature or precision of the measurements. Exemplary degrees of error are within 20 percent (%), typically, within 10%, and more typically, within 5% of a given value or range of values.

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Therapeutic Methods

The invention is based, in part, on methods of treating a patient having a disorder associated with BCMA expression (e.g. BCMA-expressing B-cell cancers, such as multiple myeloma) using dose-escalation dosing regimens with multispecific (e.g. bispecific) antibodies that bind to CD3 and BCMA. The methods are expected to reduce or inhibit unwanted treatment effects, such as cytokine release syndrome (CRS), thereby treating the patient while achieving a more favorable benefit-risk profile. In certain embodiments, the “subject” or “patient” is a human.

As used herein, a “disorder associated with BCMA expression” is a plasma cell disorder or a B cell disorder which correlates with enhanced BCMA expression. Plasma cell disorders include BCMA-expressing B-cell cancer, plasmacytoma, plasma cell leukemia, multiple myeloma, macroglobulinemia, amyloidosis, Waldenstrom's macroglobulinemia, solitary bone plasmacytoma, extramedullar plasmacytoma, osteosclerotic myeloma (POEMS Syndrome) and heavy chain diseases as well as the clinically unclear monoclonal gammopathy of undetermined significance/smoldering multiple myeloma.

In some embodiments, the B cell disorder is a BCMA-expressing B-cell cancer, such as multiple myeloma. Multiple myeloma is a plasma cell malignancy characterized by a monoclonal expansion and accumulation of abnormal plasma cells in the bone marrow compartment. Multiple myeloma also involves circulating clonal plasma cells with same IgG gene rearrangement and somatic hypermutation. Multiple myeloma arises from an asymptomatic, premalignant condition called monoclonal gammopathy of unknown significance (MGUS), characterized by low levels of bone marrow plasma cells and a monoclonal protein. Multiple myeloma cells proliferate at low rate.

Multiple myeloma results from a progressive occurrence of multiple structural chromosomal changes (e.g. unbalanced translocations). Multiple myeloma involves the mutual interaction of malignant plasma cells and bone marrow microenvironment (e.g. normal bone marrow stromal cells). Clinical signs of active multiple myeloma include monoclonal antibody spike, plasma cells overcrowding the bone marrow, lytic bone lesions and bone destruction resulting from overstimulation of osteoclasts (Dimopulos & Terpos, Ann Oncol 2010; 21 suppl 7: vii143-150).

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. Alternatively, the pharmacologic and/or physiologic effect may be prophylactic, i.e., the effect completely or partially prevents a disease or symptom thereof.

Thus, in one aspect, the present invention provides a method for treating a disorder associated with BCMA expression (e.g. BCMA-expressing B-cell cancers, such as multiple myeloma) in a patient (e.g. a human), wherein the treatment comprises the administration of a multispecific (e.g. bispecific) antibody that binds to BCMA and CD3 in a dosing regimen comprising:
(i) a starting phase, wherein one or more starting doses of the multispecific (e.g. bispecific) antibody are administered to the patient; and
(ii) a maintenance phase, wherein a first maintenance dose of the multispecific (e.g. bispecific) antibody is administered to the patient, optionally followed by at least one additional maintenance dose of the multispecific (e.g. bispecific) antibody;
   wherein each maintenance dose is greater than the one or more starting doses.

In another aspect, the present invention provides a multispecific (e.g. bispecific) antibody that binds to BCMA and CD3 for use in treating a disorder associated with BCMA expression (e.g. BCMA-expressing B-cell cancers, such as multiple myeloma) in a patient (e.g. a human), wherein the treatment comprises the administration of the multispecific (e.g. bispecific) antibody in a dosing regimen which comprises:
(i) a starting phase, wherein one or more starting doses of the multispecific (e.g. bispecific) antibody are administered to the patient; and
(ii) a maintenance phase, wherein a first maintenance dose of the multispecific (e.g. bispecific) antibody is administered to the patient, optionally followed by at least one additional maintenance dose of the multispecific (e.g. bispecific) antibody;

wherein each maintenance dose is greater than the one or more starting doses.

Administration of the starting dose of the multispecific (e.g. bispecific) antibody significantly reduces toxicity due to attenuation of cytokine release.

In some embodiments, the starting phase comprises a single fixed dose. In some embodiments, the starting dose of the multispecific (e.g. bispecific) antibody is a single fixed dose of about 1.5 mg to 4.5 mg; from about 2 mg to 4 mg; from about 2.5 mg to 3.5 mg, e.g. about 3 mg. In some embodiments, the starting dose of the multispecific (e.g. bispecific) antibody is a single fixed dose of about 4.5 mg to 7.5 mg; from about 5 mg to 6 mg; from about 5.5 mg to 6.5 mg, e.g. about 6 mg.

In other embodiments, the starting phase comprises two or more starting starting doses of the same concentration. In embodiments in which the starting doses are administered as two or more doses of the same concentration, the starting dose of the multispecific (e.g. bispecific) antibody may be administered at a fixed dose of about 1.5 mg to 4.5 mg, from about 2 mg to 4 mg, from about 2.5 mg to 3.5 mg, e.g. about 3 mg. Alternatively, the starting dose of the multispecific (e.g. bispecific) antibody may be administered at a fixed dose of about 1.5 mg to 4.5 mg, from about 2 mg to 4 mg, from about 2.5 mg to 3.5 mg, e.g. about 6 mg.

If the patient develops an adverse event (e.g. CRS or infection) following administration of a starting dose (e.g. first starting dose) of the multispecific (e.g. bispecific) antibody, the subsequent starting dose (e.g. second starting dose) may be administered to the patient up to 12 weeks after the starting dose that triggered the adverse event. In some embodiments, the subsequent starting dose may be administered up to 10 weeks after, up to 8 weeks after, up to 6 weeks after, up to 4 weeks after, up to two weeks after, e.g. up to one week after the starting dose that triggered the adverse event. In some embodiments, the subsequent starting dose may be of the same concentration or lower concentration than the starting dose that triggered the adverse event.

If the patient develops an adverse event (e.g. CRS or infection) following administration of the last starting dose of the starting phase, the starting phase may comprise an additional starting dose administered to the patient up to 12 weeks after the starting dose that triggered the adverse event. In some embodiments, the additional starting dose may be administered up to 10 weeks after, up to 8 weeks after, up to 6 weeks after, up to 4 weeks after, up to two weeks after, e.g. up to one week after the starting dose that triggered the adverse event. In some embodiments, the additional starting dose may be of the same concentration or lower concentration than the starting dose that triggered the adverse event.

In some embodiments, the first maintenance dose of the multispecific (e.g. bispecific) antibody may be administered at a fixed dose of about 4.5 mg to 7.5 mg; from about 5 mg to 6 mg; from about 5.5 mg to 6.5 mg, e.g. about 6 mg. Thus, in some embodiments, the starting dose of the multispecific (e.g. bispecific) antibody is a (e.g. single) fixed dose of about 3 mg and the first maintenance dose of the multispecific (e.g. bispecific) antibody is a fixed dose of about 6 mg.

In some embodiments, the first maintenance dose of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg. Thus, in some embodiments, the starting dose of the multispecific (e.g. bispecific) antibody is a (e.g. single) fixed dose of about 6 mg and the first maintenance dose of the multispecific (e.g. bispecific) antibody is a fixed dose of about 10 mg.

In some embodiments, the maintenance phase comprises two or more maintenance doses of the same concentration or of escalating concentration.

In some embodiments, the maintenance phase comprises two or more maintenance doses of about 4.5 mg to about 25 mg, preferably of about 4.5 mg to about 11.5 mg. In some embodiments, the maintenance phase comprises two or more maintenance doses of about 4.5 mg to about 7.5 mg; about 5 mg to about 6 mg; about 5.5 mg to about 6.5 mg, e.g. about 6 mg. In some embodiments, the maintenance phase comprises two or more maintenance doses of about 8.5 mg to about 11.5 mg; about 9 mg to about 11 mg; about 9.5 mg to about 10.5 mg, e.g. about 10 mg. In some embodiments, the maintenance phase comprises two or more maintenance doses of about 6 mg to about 11.5 mg, about 6.5 mg to about 11 mg, about 7 mg to about 10.5 mg, e.g. about 7.5 mg to about 10 mg e.g. about 10 mg. In some embodiments, the maintenance phase comprises two or more maintenance doses of about 18.5 mg to 21.5 mg; from about 19 mg to 21 mg; from about 19.5 mg to 20.5 mg, e.g. about 20 mg.

In embodiments in which the maintenance doses are administered as two or more doses of the same concentration, the maintenance dose of the multispecific (e.g. bispecific) antibody may be administered at a fixed dose of about 4.5 mg to 7.5 mg; from about 5 mg to 6 mg; from about 5.5 mg to 6.5 mg, e.g. about 6 mg. Thus, in some embodiments, the starting dose of the multispecific (e.g. bispecific) antibody is a (e.g. single) fixed dose of about 3 mg and the maintenance dose of the multispecific (e.g. bispecific) antibody is a fixed dose of about 6 mg.

In embodiments in which the maintenance doses are administered as two or more doses of the same concentration, the maintenance dose of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg. Thus, in some embodiments, the starting dose of the multispecific (e.g. bispecific) antibody is a (e.g. single) fixed dose of about 6 mg and the maintenance dose of the multispecific (e.g. bispecific) antibody is a fixed dose of about 10 mg.

In some embodiments, the maintenance dose of the multispecific (e.g. bispecific) antibody is administered as two or more doses of escalating concentration (i.e. increasing doses). In this case, a subsequent dose can be increased by a particular increment, or by variable increments, until a maximum dose is reached, at which point administration may cease or may continue at the maximum dose. Thus, in embodiments in which the maintenance doses are administered at escalating concentrations, the first maintenance dose of the multispecific (e.g. bispecific) antibody is greater than the starting dose and a subsequent (e.g. second, third, fourth or fifth) maintenance dose of the multispecific (e.g. bispecific) antibody is greater than the first maintenance dose. For example, the first maintenance dose of the multispecific (e.g. bispecific) antibody is greater than the starting dose, the second maintenance dose(s) of the multispecific (e.g. bispecific) antibody is the same as the first maintenance dose and the third (and optionally subsequent) maintenance dose(s) of the multispecific (e.g. bispecific) antibody is greater than the second maintenance dose.

In some embodiments, the first maintenance dose of the multispecific (e.g. bispecific) antibody may be administered at a fixed dose of about 4.5 mg to 7.5 mg; from about 5 mg to 6 mg; from about 5.5 mg to 6.5 mg, e.g. about 6 mg, and a subsequent (e.g. second, third, fourth or fifth) maintenance dose of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg.

In some embodiments, the first maintenance dose of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg, and a subsequent (e.g. second, third, fourth or fifth) maintenance dose of the multispecific (e.g. bispecific) antibody may be administered at a fixed dose greater than the first maintenance dose.

In some embodiments, the second maintenance dose of the multispecific (e.g. bispecific) antibody is greater than the first maintenance dose. In some embodiments, the first maintenance dose of the multispecific (e.g. bispecific) antibody may be administered at a fixed dose of about 4.5 mg to 7.5 mg; from about 5 mg to 6 mg; from about 5.5 mg to 6.5 mg e.g. about 6 mg, and the second maintenance dose of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg. Thus, in some embodiments, the starting dose is a (e.g. single) fixed dose of about 3 mg, the first maintenance dose of the is a fixed dose of about 6 mg and a second maintenance dose is a fixed dose of about 10 mg. In some embodiments, subsequent (e.g. third, fourth or fifth) maintenance dose of the multispecific (e.g. bispecific) antibody may be the same or greater than the second maintenance dose.

In some embodiments, the first maintenance dose of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg, and the second maintenance dose of the multispecific (e.g. bispecific) antibody may be administered at a fixed dose greater than the first maintenance dose. Thus, in some embodiments, the starting dose is a (e.g. single) fixed dose of about 6 mg, the first maintenance dose of the is a fixed dose of about 10 mg and a second maintenance dose is a fixed dose greater than the first maintenance dose. In some embodiments, subsequent (e.g. third, fourth or fifth) maintenance dose of the multispecific (e.g. bispecific) antibody may be the same or greater than the second maintenance dose.

In some embodiments, the maintenance dose of the multispecific (e.g. bispecific) antibody is administered as two concentrations: a first concentration and a maximum dose concentration. In some embodiments, the first maintenance dose may be administered at a fixed dose of about 4.5 mg to 7.5 mg; from about 5 mg to 7 mg; from about 5.5 mg to 6.5 mg, e.g. about 6 mg, and a subsequent (e.g. second) maintenance dose of the multispecific (e.g. bispecific) antibody may be administered at the maximum dose concentration. In some embodiments, the maximum dose concentration is a fixed dose of about 8.5 mg to 11.5 mg; from about 9 mg to 11 mg; from about 9.5 mg to 10.5 mg, e.g. about 10 mg. Thus, in some embodiments, the starting dose is a (e.g. single) fixed dose of about 3 mg, the first maintenance dose of the is a fixed dose of about 6 mg and a subsequent (e.g. second) maintenance dose is a maximum dose, which is a fixed dose of about 10 mg. If the maximum maintenance dose is administered subcutaneously, the maximum dose concentration may be a fixed dose of about 18.5 mg to 21.5 mg; from about 19 mg to 21 mg; from about 19.5 mg to 20.5 mg, e.g. about 20 mg.

In some embodiments, the first maintenance dose may be administered at a fixed dose of about 8.5 mg to 11.5 mg; from about 9 mg to 11 mg; from about 9.5 mg to 10.5 mg, e.g. about 10 mg, and the a subsequent (e.g. second) maintenance dose of the multispecific (e.g. bispecific) antibody may be administered at the maximum dose concentration. Thus, in some embodiments, the starting dose is a (e.g. single) fixed dose of about 6 mg, the first maintenance dose of the is a fixed dose of about 10 mg and a subsequent (e.g. second) maintenance dose is a maximum dose, which is greater than the first maintenance dose.

In some embodiments, the first maintenance dose of the multispecific (e.g. bispecific) antibody is administered to the patient 1-21 days, e.g. 1, 2, 3, 4, 5, 6, 7, 8, 9, 10, 11, 12, 13 or 14 days, after the starting dose. In some embodiments, the first maintenance dose of the multispecific (e.g. bispecific) antibody may be administered to the patient 2 days, after the starting dose. In some embodiments, the first maintenance dose of the multispecific (e.g. bispecific) antibody may be administered to the patient 3 days after the starting dose. In some embodiments, the first maintenance dose of the multispecific (e.g. bispecific) antibody may be administered to the patient 7 days after the starting dose. In some embodiments, the first maintenance dose of the multispecific (e.g. bispecific) antibody may be administered to the patient 14 days after the starting dose.

In some embodiments, the second maintenance dose of the multispecific (e.g. bispecific) antibody is administered to the patient 1-21 days, e.g. 2, 4, 7 or 14 days, after the first maintenance dose. Thus, in embodiments in which the first maintenance dose 2 days after the starting dose, the second maintenance dose may be administered 2 days after the first maintenance dose, and optionally a third maintenance dose may be administered 3 days after the second maintenance dose.

In embodiments in which the first maintenance dose is administered 3 days after the starting dose, the second maintenance dose may be administered 4 days after the first maintenance dose. In embodiments in which the first maintenance dose is administered 7 days after the starting dose, the second (and optionally subsequent) maintenance dose(s) may be administered 7 days after the first maintenance dose. In embodiments in which the first maintenance dose is administered 14 days after the starting dose, the second (and optionally subsequent) maintenance dose(s) may be administered 14 days after the first maintenance dose.

In some embodiments of any aspect of the invention, if the patient develops an adverse event (e.g. CRS or infection) following administration of a maintenance dose (e.g. first, second, third or subsequent maintenance dose) of the multispecific (e.g. bispecific) antibody, the next maintenance dose may be administered to the patient up to 12 weeks after the maintenance dose that triggered the adverse event. In some embodiments, the next maintenance starting dose may be administered up to 10 weeks after, up to 8 weeks after, up to 6 weeks after, up to 4 weeks after, up to two weeks after, e.g. up to one week after the starting dose that triggered the adverse event. In some embodiments, the next maintenance dose may be of the same concentration or lower concentration than the maintenance dose that triggered the adverse event.

In some embodiments of any aspect of the invention, the third and subsequent maintenance doses are administered at about a once weekly or longer dosing interval. As used herein, a “dosing interval” means the amount of time that elapses between multiple doses being administered to a patient. If an adverse event (e.g. CRS or infection) occurs following administration of a maintenance dose (e.g. third or subsequent maintenance dose) of the multispecific (e.g. bispecific) antibody, the dosing interval may reset on the day the next maintenance dose is administered to the patient.

In some embodiments of any aspect of the invention, the dosing interval for the third and subsequent maintenance doses may be about once weekly. As used herein, a “weekly dosing interval” includes every 5-9, 6-9, 7-9, 5-8, 5-7, 6-8, 6-7, 7-8, preferably 7 days. In some embodiments, the dosing interval for the third and subsequent maintenance doses may be about once biweekly. As used herein, a “biweekly dosing interval” includes every 12-16, 13-16, 14-16, 12-15, 12-14, 13-15, 13-14, 14-15, preferably 14 days. In some embodiments, the dosing interval for the third and subsequent maintenance dose may be about once every three weeks. As used herein, a “three week dosing interval” includes every 19-23, 20-23, 21-23, 19-22, 19-21, 20-22, 20-21, 21-22, preferably 21 days. In some embodiments, the dosing interval for the third and subsequent maintenance dose may be about once every four weeks. As used herein, a “four week dosing interval” includes every 26-30, 27-30, 28-30, 26-29, 26-28, 27-29, 27-28, 28-29, preferably 28 days. In some embodiments, the dosing interval for the third and subsequent maintenance doses may be about once monthly.

In some embodiments of any aspect of the invention, the dosing interval for the third and subsequent maintenance dose may be a combination of one or more of a weekly dosing interval, a biweekly dosing interval, a three week dosing interval and a four week dosing interval. In some embodiments, the dosing interval for the third and subsequent maintenance dose may be a combination of a weekly dosing interval, a biweekly dosing interval, and a four week dosing interval.

In some embodiments of any aspect of the invention, the third and subsequent maintenance doses are administered in a weekly dosing interval (e.g. every 7 days), then a biweekly dosing interval (e.g. every 14 days), then a three week dosing interval (e.g. every 21 days) and then a four week dosing interval (e.g. every 28 days). In some embodiments, the third and subsequent maintenance doses are administered in a weekly dosing interval (e.g. every 7 days), then a biweekly dosing interval (e.g. every 14 days) and then a four week dosing interval (e.g. every 28 days).

In some embodiments of any aspect of the invention, the treatment comprises at least one treatment cycle of 28 days. As used herein, a “treatment cycle” is 28 days. If a starting dose is administered beyond day 28 of the first treatment cycle as a result of an adverse event (e.g. CRS or infection), the first treatment cycle may restart on the day the starting dose is administered to the patient. If a maintenance dose is administered beyond day 28 of the current treatment cycle as a result of an adverse event (e.g. CRS or infection), the next treatment cycle may begin on the day the maintenance dose is administered to the patient. In some embodiments, the treatment comprises a first treatment cycle, wherein the starting dose is administered to the patient as a fixed dose on day 1, and the maintenance doses are subsequently administered in a weekly dosing interval (e.g. every 7 days) for three consecutive weeks (e.g. on days 8, 15 and 22). The maintenance doses may continue to be administered in a weekly or longer dosing interval in subsequent treatment cycles.

In some embodiments of any aspect of the invention, the treatment comprises a second treatment cycle, wherein the maintenance doses are administered in a weekly dosing interval (e.g. on days 1, 8, 15 and 22). In further embodiments, the patient remains on a weekly dosing interval for between 1-5, 1-3, 1-2, 2-3 further treatment cycles, preferably 2 further treatment cycles (in addition to the first treatment cycle). In some embodiments, the treatment comprises a second and third treatment cycle, wherein the maintenance doses are administered in a weekly dosing interval (e.g. on days 1, 8, 15 and 22).

In some embodiments of any aspect of the invention, the maintenance doses may be administered in a biweekly dosing interval in treatment cycles (e.g. on days 1 and 15) after completion of the weekly treatment cycle(s). In further embodiments, the patient remains on a biweekly dosing interval for between 1-5, 1-3, 1-2, 2-3 biweekly treatment cycles, preferably 3 biweekly treatment cycles. In some embodiments, the treatment comprises a fourth, fifth and sixth treatment cycle, wherein the maintenance doses are administered in a biweekly dosing interval (e.g. on days 1 and 15).

In some embodiments of any aspect of the invention, the maintenance doses may be administered in a three week dosing interval in subsequent treatment cycles (e.g. subsequent cycles follow the sequence (a), (b) and (c), wherein the maintenance doses are administered on days 1 and 22 of cycle (a), on day 15 of cycle (b), and on day 8 of cycle (c)) after completion of the biweekly treatment cycle(s). In further embodiments, the patient remains on a three week dosing interval for 1, 2 or 3 treatment cycles.

In some embodiments of any aspect of the invention, the maintenance doses may be administered in a four week dosing interval in subsequent treatment cycles (e.g. on day 1) after completion of the biweekly treatment cycle(s). In alternative embodiments, the maintenance doses may be administered in a four week dosing interval in subsequent treatment cycles (e.g. on day 1) after completion of the three week treatment cycle(s). In further embodiments, the patient remains on a four week dosing interval for at least one cycle. Some patients continue to receive treatment for the rest of their lives.

In some embodiments, the treatment comprises:
(i) a first treatment cycle, wherein the starting dose is administered on day 1, and the maintenance doses are administered on days 8, 15 and 22;
(ii) a second and third treatment cycle, wherein the maintenance doses are administered in a weekly dosing interval (e.g. on days 1, 8, 15 and 22);
(iii) a fourth to sixth treatment cycle, wherein the maintenance doses are administered in a biweekly dosing interval (e.g. on days 1 and 15); and
(iv) a seventh and subsequent cycle, wherein the maintenance doses are administered in a four week dosing interval (e.g. on day 1)

In some embodiments, the multispecific (e.g. bispecific) antibody (e.g. “42-TCBcv”) may be administered to the patient in accordance with the regimen set out in Table 1.

TABLE 1

| Cycle 1 | Cycle 2-3 | Cycles 4-6 | Cycles 7+ |
|---|---|---|---|
| Starting dose on day 1 | Maintenance | Maintenance | Maintenance |
| Maintenance dose on | dose on days | dose on | dose on day 1 |
| days 8, 15, 22 | 1, 8, 15, 22 | days 1, 15 | |

In alternative embodiments, the treatment comprises a first treatment cycle, wherein the starting dose is administered to the patient as a fixed dose on day 1, the first maintenance dose is administered 3 days after the starting dose (e.g. on day 4), the second maintenance dose is administered 4 days after the first maintenance dose (e.g. on day 8), and the third and fourth maintenance doses are administered in a weekly interval (e.g. on days 15 and 22). The maintenance doses may continue to be administered in a weekly or longer dosing interval in subsequent treatment cycles.

In some embodiments of any aspect of the invention, the treatment comprises a second treatment cycle, wherein the maintenance doses are administered in a weekly dosing interval (e.g. on days 1, 8, 15 and 22). In further embodiments, the patient remains on a weekly dosing interval for between 1-5, 1-3, 1-2, 2-3 further treatment cycles, preferably 2 further treatment cycles (in addition to the first treatment cycle). In some embodiments, the treatment comprises a second and third treatment cycle, wherein the maintenance doses are administered in a weekly dosing interval (e.g. on days 1, 8, 15 and 22).

In some embodiments of any aspect of the invention, the maintenance doses may be administered in a biweekly dosing interval in treatment cycles (e.g. on days 1 and 15) after completion of the weekly treatment cycle(s). In further embodiments, the patient remains on a biweekly dosing interval for between 1-5, 1-3, 1-2, 2-3 weekly treatment cycles, preferably 3 biweekly treatment cycles. In some embodiments, the treatment comprises a fourth, fifth and sixth treatment cycle, wherein the maintenance doses are administered in a biweekly dosing interval (e.g. on days 1 and 15).

In some embodiments of any aspect of the invention, the maintenance doses may be administered in a four week dosing interval in subsequent treatment cycles (e.g. on day 1) after completion of the biweekly treatment cycle(s). In further embodiments, the patient remains on a four week dosing interval for at least one cycle. Some patients continue to receive treatment for the rest of their lives.

In some embodiments, the treatment comprises:
(i) a first treatment cycle, wherein the starting dose is administered on day 1, and the maintenance doses are administered on days 4, 8, 15 and 22;
(ii) a second and third treatment cycle, wherein the maintenance doses are administered in a weekly dosing interval (e.g. on days 1, 8, 15 and 22);
(iii) a fourth to sixth treatment cycle, wherein the maintenance doses are administered in a biweekly dosing interval (e.g. on days 1 and 15); and
(iv) a seventh and subsequent cycle, wherein the maintenance doses are administered in a four week dosing interval (e.g. on day 1)

In some embodiments, the multispecific (e.g. bispecific) antibody (e.g. “42-TCBcv”) may be administered to the patient in accordance with the regimen set out in Table 2.

TABLE 2

| Cycle 1 | Cycle 2-3 | Cycles 4-6 | Cycles 7+ |
|---|---|---|---|
| Starting dose on day 1 | Maintenance | Maintenance | Maintenance |
| Maintenance dose on | dose on days | dose on | dose on day 1 |
| days 4, 8, 15, 22 | 1, 8, 15, 22 | days 1, 15 | |

In alternative embodiments, the treatment comprises a first treatment cycle, wherein the starting dose is administered to the patient as a fixed dose on day 1, the first maintenance dose is administered 2 days after the starting dose (e.g. on day 3), the second maintenance dose is administered 2 days after the first maintenance dose (e.g. on day 5), the third maintenance dose is administered 3 days after the first maintenance dose (e.g. on day 8), and the fourth and fifth maintenance doses are administered in a weekly interval (e.g. on days 15 and 22). The maintenance doses may continue to be administered in a weekly or longer dosing interval in subsequent treatment cycles.

In some embodiments of any aspect of the invention, the treatment comprises a second treatment cycle, wherein the maintenance doses are administered in a weekly dosing interval (e.g. on days 1, 8, 15 and 22). In further embodiments, the patient remains on a weekly dosing interval for between 1-5, 1-3, 1-2, 2-3 further treatment cycles, preferably 2 further treatment cycles (in addition to the first treatment cycle). In some embodiments, the treatment comprises a second and third treatment cycle, wherein the maintenance doses are administered in a weekly dosing interval (e.g. on days 1, 8, 15 and 22).

In some embodiments of any aspect of the invention, the maintenance doses may be administered in a biweekly dosing interval in treatment cycles (e.g. on days 1 and 15) after completion of the weekly treatment cycle(s). In further embodiments, the patient remains on a biweekly dosing interval for between 1-5, 1-3, 1-2, 2-3 weekly treatment cycles, preferably 3 biweekly treatment cycles. In some embodiments, the treatment comprises a fourth, fifth and sixth treatment cycle, wherein the maintenance doses are administered in a biweekly dosing interval (e.g. on days 1 and 15).

In some embodiments of any aspect of the invention, the maintenance doses may be administered in a four week dosing interval in subsequent treatment cycles (e.g. on day 1) after completion of the biweekly treatment cycle(s). In further embodiments, the patient remains on a four week dosing interval for at least one cycle. Some patients continue to receive treatment for the rest of their lives.

In some embodiments, the treatment comprises:
(i) a first treatment cycle, wherein the starting dose is administered on day 1, and the maintenance doses is administered on days 3, 5, 8, 15 and 22;
(ii) a second and third treatment cycle, wherein the maintenance doses are administered in a weekly dosing interval (e.g. on days 1, 8, 15 and 22);
(iii) a fourth to sixth treatment cycle, wherein the maintenance doses are administered in a biweekly dosing interval (e.g. on days 1 and 15); and
(iv) a seventh and subsequent cycle, wherein the maintenance doses are administered in a four week dosing interval (e.g. on day 1)

In some embodiments, the multispecific (e.g. bispecific) antibody (e.g. “42-TCBcv”) may be administered to the patient in accordance with the regimen set out in Table 3.

TABLE 3

| Cycle 1 | Cycle 2-3 | Cycles 4-6 | Cycles 7+ |
|---|---|---|---|
| Starting dose on day 1 | Maintenance dose on | Maintenance dose on | Maintenance dose on |
| Maintenance dose on | days 1, 8, 15, 22 | days 1, 15 | day 1 |
| days 3, 5, 8, 15, 22 | | | |

In some embodiments of the regimens set out in Table 1, Table 2 or Table 3, the maintenance doses of the multispecific (e.g. bispecific) antibody are administered as two or more doses of the same concentration. In some embodiments, the starting dose of the multispecific (e.g. bispecific) antibody is a fixed dose of about 1.5 mg to 4.5 mg; from about 2 mg to 4 mg; from about 2.5 mg to 3.5 mg, e.g. about 3 mg. In some embodiments, the first and subsequent maintenance doses of the multispecific (e.g. bispecific) antibody are a fixed dose of about 4.5 mg to 7.5 mg; from about 5 mg to 7 mg; from about 5.5 mg to 6.5 mg, e.g. about 6 mg. In some embodiments, the starting dose of the multispecific (e.g. bispecific) antibody is a (e.g. single) fixed dose of about 3 mg and the first and subsequent maintenance doses of the multispecific (e.g. bispecific) antibody are a fixed dose of about 6 mg.

In some embodiments of the regimens set out in Table 1, Table 2 or Table 3, the maintenance doses of the multispecific (e.g. bispecific) antibody are administered as two or more doses of the same concentration. In some embodiments, the starting dose of the multispecific (e.g. bispecific) antibody is a fixed dose of about 4.5 mg to 7.5 mg; from about 5 mg to 6 mg; from about 5.5 mg to 6.5 mg, e.g. about 6 mg. In some embodiments, the first and subsequent maintenance doses of the multispecific (e.g. bispecific) antibody are a fixed dose of about 8.5 mg to 11.5 mg; from about 9 mg to 11 mg; from about 9.5 mg to 10.5 mg, e.g. about 10 mg. In some embodiments, the starting dose of the multispecific (e.g. bispecific) antibody is a fixed dose of about 6 mg and the first and subsequent maintenance doses of the multispecific (e.g. bispecific) antibody are a fixed dose of about 10 mg.

In some embodiments, the multispecific (e.g. bispecific) antibody (e.g. “42-TCBcv”) may be administered to the patient in accordance with the regiment set out in Table 4.

TABLE 4

| Cycle 1 | Cycle 2-3 | Cycles 4-6 | Cycles 7+ |
|---|---|---|---|
| Starting dose on day 1 | Maximum maintenance | Maximum maintenance | Maximum maintenance |
| (e.g. about 3 or 6 mg) | dose on days 1, 8, 15, | dose on days 1, 15 (e.g. | dose on day 1 (e.g. |
| Maximum maintenance | 22 (e.g. about 6 or 10 | about 6 or 10 mg) | about 6 or 10 mg) |
| dose on days 8, 15, 22 | mg) | | |
| (e.g. about 6 or 10 mg) | | | |

In some embodiments, the multispecific (e.g. bispecific) antibody (e.g. “42-TCBcv”) may be administered to the patient in accordance with the regiment set out in Table 5.

TABLE 5

| Cycle 1 | Cycle 2-3 | Cycles 4-6 | Cycles 7+ |
|---|---|---|---|
| Starting dose on day 1 | Maximum maintenance | Maximum maintenance | Maximum maintenance |
| (e.g. about 3 or 6 mg) | dose on days 1, 8, 15, | dose on days 1, 15 (e.g. | dose on day 1 (e.g. |
| Maximum maintenance | 22 (e.g. about 6 or 10 | about 6 or 10 mg) | about 6 or 10 mg) |
| dose on days 4, 8, 15, | mg) | | |
| 22 (e.g. about 6 or 10 | | | |
| mg) | | | |

In some embodiments, the multispecific (e.g. bispecific) antibody (e.g. “42-TCBcv”) may be administered to the patient in accordance with the regiment set out in Table 6.

TABLE 6

| Cycle 1 | Cycle 2-3 | Cycles 4-6 | Cycles 7+ |
|---|---|---|---|
| Starting dose on day 1 | Maximum maintenance | Maximum maintenance | Maximum maintenance |
| (e.g. about 3 or 6 mg) | dose on days 1, 8, 15, | dose on days 1, 15 (e.g. | dose on day 1 (e.g. |
| Maximum maintenance | 22 (e.g. about 6 or 10 | about 6 or 10 mg) | about 6 or 10 mg) |
| dose on days 3, 5, 8, | mg) | | |
| 15, 22 (e.g. about 6 or | | | |
| 10 mg) | | | |

In some embodiments of the regimens set out in Table 1, Table 2 or Table 3, the maintenance doses of the multispecific (e.g. bispecific) antibody are administered as two or more doses of escalating concentration.

In some embodiments, the starting dose of the multispecific (e.g. bispecific) antibody is a fixed dose of about 1.5 mg to 4.5 mg; from about 2 mg to 4 mg; from about 2.5 mg to 3.5 mg, e.g. about 3 mg. In some embodiments, the first maintenance dose may be administered at a fixed dose of about 4.5 mg to 7.5 mg; from about 5 mg to 7 mg; from about 5.5 mg to 6.5 mg, e.g. about 6 mg, and the second (and optionally subsequent) maintenance dose(s) of the multispecific (e.g. bispecific) antibody may be administered at a fixed dose of about 8.5 mg to 11.5 mg; from about 9 mg to 11 mg; from about 9.5 mg to 10.5 mg, e.g. about 10 mg. Thus, in some embodiments, the starting dose of the multispecific (e.g. bispecific) antibody is a (e.g. single) fixed dose of about 3 mg, the first maintenance dose of the multispecific (e.g. bispecific) antibody is a fixed dose of about 6 mg and the second (and optionally subsequent) maintenance dose(s) of the multispecific (e.g. bispecific) antibody is a fixed dose of about 10 mg.

In some embodiments, the starting dose of the multispecific (e.g. bispecific) antibody is a fixed dose of about 4.5 mg to 7.5 mg; from about 5 mg to 7 mg; from about 5.5 mg to 6.5 mg, e.g. about 6 mg. In some embodiments, the first maintenance dose may be administered at a fixed dose of about 8.5 mg to 11.5 mg; from about 9 mg to 11 mg; from about 9.5 mg to 10.5 mg, e.g. about 10 mg, and the second (and optionally subsequent) maintenance dose(s) of the multispecific (e.g. bispecific) antibody may be administered at a fixed dose of greater than the first maintenance dose. Thus, in some embodiments, the starting dose of the multispecific (e.g. bispecific) antibody is a fixed dose of about 6 mg, the first maintenance dose of the multispecific (e.g. bispecific) antibody is a fixed dose of about 10 mg and the second (and optionally subsequent) maintenance dose(s) of the multispecific (e.g. bispecific) antibody is greater than the first maintenance dose.

In some embodiments, the multispecific (e.g. bispecific) antibody (e.g. “42-TCBcv”) may be administered to the patient in accordance with the regiment set out in Table 7.

TABLE 7

| Cycle 1 | Cycle 2-3 | Cycles 4-6 | Cycles 7+ |
|---|---|---|---|
| Starting dose on day 1 | Maximum maintenance | Maximum maintenance | Maximum maintenance |
| (e.g. 3 or 6 mg) | dose on days 1, 8, 15, | dose on days 1, 15 (e.g. | dose on day 1 (e.g. 10 |
| First maintenance dose | 22 (e.g. 10 or 10+ mg) | 10 or 10+ mg) | or 10+ mg) |
| on day 8 (e.g. 6 or 10 | | | |
| mg) | | | |
| Maximum maintenance | | | |
| dose on days 15, 22 | | | |
| (e.g. 10 or 10+ mg) | | | |

In some embodiments, the multispecific (e.g. bispecific) antibody (e.g. “42-TCBcv”) may be administered to the patient in accordance with the regiment set out in Table 8.

TABLE 8

| Cycle 1 | Cycle 2-3 | Cycles 4-6 | Cycles 7+ |
|---|---|---|---|
| Starting dose on day 1 | Maximum maintenance | Maximum maintenance | Maximum maintenance |
| (e.g. 3 or 6 mg) | dose on days 1, 8, 15, | dose on days 1, 15 (e.g. | dose on day 1 (e.g. 10 |
| First maintenance dose | 22 (e.g. 10 or 10+ mg) | 10 or 10+ mg) | or 10+ mg) |
| on day 4 (e.g. 6 or 10 | | | |
| mg) | | | |
| Maximum maintenance | | | |
| dose on days 8, 15, 22 | | | |
| (e.g. 10 or 10+ mg) | | | |

In some embodiments, the multispecific (e.g. bispecific) antibody (e.g. “42-TCBcv”) may be administered to the patient in accordance with the regiment set out in Table 9.

TABLE 9

| Cycle 1 | Cycle 2-3 | Cycles 4-6 | Cycles 7+ |
|---|---|---|---|
| Starting dose on day 1 | Maximum maintenance | Maximum maintenance | Maximum maintenance |
| (e.g. 3 or 6 mg) | dose on days 1, 8, 15, | dose on days 1, 15 (e.g. | dose on day 1 (e.g. 10 |
| First maintenance dose | 22 (e.g. 10 or 10+ mg) | 10 or 10+ mg) | or 10+ mg) |
| on day 3 (e.g. 6 or 10 | | | |
| mg) | | | |
| Maximum maintenance | | | |
| dose on days 5, 8, 15, | | | |
| 22 (e.g. 10 or 10+ mg) | | | |

In some embodiments of any aspect of the invention, the multispecific (e.g. bispecific) antibody is administered intravenously or subcutaneously. In this regard, data (not shown) suggest that subcutaneous administration of the multispecific (e.g. bispecific) antibody (e.g. “42-TCBcv”) has comparable bioavailability to intravenous administration.

In some embodiments, the starting dose and the first maintenance dose of the multispecific (e.g. bispecific) antibody may be administered intravenously, and a subsequent (e.g. second, third, fourth or fifth) maintenance dose of the multispecific (e.g. bispecific) may be administered subcutaneously.

In some embodiments, the multispecific (e.g. bispecific) antibody (e.g. “42-TCBcv”) may be administered to the patient in accordance with any of the regimens set out in Tables 1-9, wherein the starting dose and the first maintenance dose of the multispecific (e.g. bispecific) antibody may be administered intravenously, and a subsequent (e.g. second, third, fourth or fifth) maintenance dose of the multispecific (e.g. bispecific) may be administered subcutaneously.

In some embodiments, the multispecific (e.g. bispecific) antibody (e.g. “42-TCBcv”) may be administered to the patient in accordance with any of the regimens set out in Tables 1-9, wherein cycles 1-2 may be administered intravenously, and cycles 3+ may be administered subcutaneously.

If the dose of the multispecific (e.g. bispecific) antibody (e.g. “42-TCBcv”) is administered subcutaneously, the maximum dose concentration may be a fixed dose of about 18.5 mg to 21.5 mg; from about 19 mg to 21 mg; from about 19.5 mg to 20.5 mg, e.g. about 20 mg.

In preferred embodiments of any aspect of the invention, the multispecific (e.g. bispecific) antibody is administered intravenously.

High Dose of Multispecific (e.g. Bispecific) Antibody

In another aspect, the present invention provides a method for treating a disorder associated with BCMA expression (e.g. BCMA-expressing B-cell cancers, such as multiple myeloma) in a patient (e.g. a human), wherein the treatment comprises the administration of a first maintenance dose of a multispecific (e.g. bispecific) antibody that binds to BCMA and CD3 to the patient, optionally followed by one or more additional maintenance dose(s) of the multispecific (e.g. bispecific) antibody.

In another aspect, the present invention provides a multispecific (e.g. bispecific) antibody that binds to BCMA and CD3 for use in treating a disorder associated with BCMA expression (e.g. BCMA-expressing B-cell cancers, such as multiple myeloma) in a patient (e.g. a human), wherein the treatment comprises the administration of a first maintenance dose of the multispecific (e.g. bispecific) antibody to the patient, optionally followed by one or more additional maintenance dose(s) of the multispecific (e.g. bispecific) antibody.

In some embodiments, the first maintenance dose may have a concentration of about 4.5 mg to about 11.5 mg (referred to herein as a ‘high dose of the multispecific (e.g. bispecific) antibody’). In some embodiments, the first maintenance dose is a fixed dose of more than 6 mg, more than 6.5 mg, more than 7 mg, e.g. more than 7.5 mg. In some embodiments, the first maintenance dose is a fixed dose of about 6 mg to about 11.5 mg, from about 6.5 mg to about 11 mg, from about 7 mg to about 10.5 mg, e.g. from about 7.5 mg to about 10 mg. In some embodiments, the first maintenance dose is a fixed dose of about 8.5 mg to 11.5 mg; from about 9 mg to about 11 mg; from about 9.5 mg to about 10.5 mg, e.g. about 10 mg. In alternative embodiments, the first maintenance dose is a fixed dose of about 4.5 mg to about 7.5 mg; from about 5 mg to about 6 mg; from about 5.5 mg to about 6.5 mg, e.g. about 6 mg.

In some embodiments, no CRS events of Grade >3 occur following administration of the first maintenance dose, preferably no CRS events of Grade >2 occur, preferably no CRS events of Grade >1 occur, preferably no CRS events of Grade 1 or higher, preferably no CRS events occur, optionally wherein the first maintenance dose is administered without dexamethasone prophylaxis.

In embodiments where the treatment comprises one or more additional maintenance dose(s), i.e. at least a second maintenance dose, of the multispecific (e.g. bispecific) antibody, the second maintenance dose may be administered to the patient 1-21 days, e.g. 1, 2, 3, 4, 5, 6, 7, 8, 9, 10, 11, 12, 13, 14, 15, 16, 17, 18, 19, 20 or 21 days, after the first maintenance dose. In some embodiments, the second maintenance dose of the multispecific (e.g. bispecific) antibody is administered to the patient 7 days after the first maintenance dose. In some embodiments, the second maintenance dose of the multispecific (e.g. bispecific) antibody is administered to the patient 14 days after the first maintenance dose. In some embodiments, no CRS events of Grade >3 occur following administration of the second maintenance dose, preferably no CRS events of Grade >2 occur, preferably no CRS events of Grade >1 occur, preferably no CRS events of Grade 1 or higher, preferably no CRS events occur, optionally wherein the second maintenance dose is administered without dexamethasone prophylaxis.

The treatment may comprise a third maintenance dose of the multispecific (e.g. bispecific) antibody administered to the patient 1-21 days, e.g. 1, 2, 3, 4, 5, 6, 7, 8, 9, 10, 11, 12, 13, 14, 15, 16, 17, 18, 19, 20 or 21 days, after the second maintenance dose. In some embodiments, the third maintenance dose of the multispecific (e.g. bispecific) antibody is administered to the patient 7 days after the second maintenance dose. In some embodiments, the third maintenance dose of the multispecific (e.g. bispecific) antibody is administered to the patient 14 days after the second maintenance dose. In some embodiments, no CRS events of Grade >3 occur following administration of the third maintenance dose, preferably no CRS events of Grade >2 occur, preferably no CRS events of Grade >1 occur, preferably no CRS events of Grade 1 or higher, preferably no CRS events occur, optionally wherein the third maintenance dose is administered without dexamethasone prophylaxis.

In some embodiments, the treatment comprises administration of further maintenance doses, e.g. fourth, fifth, sixth maintenance doses. In some embodiments in which the treatment comprises a fourth maintenance dose, the treatment comprises a first treatment cycle, optionally wherein the first maintenance dose is administered to the patient as a fixed dose on day 1, and additional maintenance doses are subsequently administered in a weekly dosing interval (e.g. every 7 days) for three consecutive weeks (e.g. on days 8, 15 and 22).

In some embodiments, the treatment comprises subsequent treatment cycles, e.g. second, third, fourth, fifth, sixth, seventh treatment cycles. In some embodiments in which the treatment comprises subsequent treatment cycles, the maintenance doses continue to be administered in a weekly or longer dosing interval in the subsequent treatment cycles.

In some embodiments, the treatment comprises:
(i) a first treatment cycle, wherein the first maintenance dose is administered on day 1, and additional maintenance doses are administered on days 8, 15 and 22;
(ii) a second and third treatment cycle, wherein the maintenance doses are administered in a weekly dosing interval (e.g. on days 1, 8, 15 and 22);
(iii) a fourth to sixth treatment cycle, wherein the maintenance doses are administered in a biweekly dosing interval (e.g. on days 1 and 15); and
(iv) a seventh and subsequent cycle, wherein the maintenance doses are administered in a four week dosing interval (e.g. on day 1).

Thus, it will be appreciated that the multispecific (e.g. bispecific) antibody (e.g. “42-TCBcv”) may be administered to the patient in accordance with the regimen set out in Table 1 with the first maintenance dose being administered in place of the “starting dose”.

In some embodiments, the one or more additional maintenance dose(s) are fixed doses of the same concentration as the first maintenance dose. If the patient develops an adverse event (e.g. CRS) following administration of a maintenance dose (e.g. first, second, third or fourth maintenance dose), the subsequent maintenance dose (e.g. second, third, fourth or fifth maintenance dose) may be of lower concentration than the maintenance dose that triggered the adverse event (e.g. CRS).

Adverse Events

In some embodiments of any aspect of the invention, the patient develops, or is at risk of developing, an adverse event associated with the administration of the multispecific (e.g. bispecific) antibody. The adverse event may be cytokine-driven toxicities (e.g. cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, bacterial infections, viral infections, and/or central nervous system (CNS) toxicities. In particular embodiments, the adverse event is CRS.

In the event that the patient develops, or is at risk of developing, an adverse event associated with the administration of the multispecific (e.g. bispecific) antibody, the treatment according to any aspect of the invention further comprises the administration of an agent capable of treating, preventing, delaying, reducing or attenuating the development or risk of development of the adverse event. The agent may be administered to the patient prior to the initiation of the treatment with the multispecific (e.g. bispecific) antibody (e.g. as a prophylaxis in order to prevent or reduce the risk of an adverse event developing) or during treatment with the multispecific (e.g. bispecific) antibody (e.g. in response to the development of an adverse event). In some embodiments, the agent comprises a steroid, such as a corticosteroid. As used herein, “corticosteroid” means any naturally occurring or synthetic steroid hormone that can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at Δ4, a C3 ketone, and a C20 ketone. Corticosteroids may have glucocorticoid and/or mineralocorticoid activity. Examples of exemplary corticosteroids include prednisolone, methylprednisolone, prednisone, triamcinolone, betamethasone, budesonide, and dexamethasone. In some embodiments, the agent is dexamethasone.

In some embodiments, the agent comprises an antagonist of a cytokine receptor or cytokine selected from among GM-CSF, IL-10, IL-10R, IL-6, IL-6 receptor (IL-6R), IFNγ, IFNGR, IL-2, IL-2R/CD25, MCP-1, CCR2, CCR4, MIPIβ, CCR5, TNFalpha, TNFR1, IL-1 (e.g. IL-1α, IL-1β, IL-1RA), and IL-1 receptor (IL-1R), wherein the antagonist is selected from an antibody or antigen-binding fragment, a small molecule, a protein or peptide and a nucleic acid. The antagonist may be an anti-IL-6 antibody and/or an anti-IL6R antibody. For example, the antagonist may be selected from tocilizumab, siltuximab, clazakizumab, sarilumab, olokizumab, elsilimomab, ALD518/BMS-945429, sirukumab (CNTO 136), CPSI-2634, ARGX-109, lenzilumab, FE301 and FM101. In some embodiments, the antagonist is tocilizumab and/or siltuximab. Alternatively, the antagonist may be an anti-IL-1 antagonist and/or an anti-IL-1R antagonist e.g. anakinra.

In some embodiments, the agent comprises a molecule that decreases the regulatory T cell (Treg) population. Agents that decrease the number of (e.g., deplete) Treg cells are known in the art and include, e.g., CD25 depletion, cyclophosphamide administration, anti-CTLA4 antibody and modulating Glucocorticoid-induced TNLR family related gene (GITR) function. GITR is a member of the TNLR superfamily that is upregulated on activated T cells, which enhances the immune system. In some embodiments, the treatment comprises the administration of cyclophosphamide.

In some embodiments, the agent capable of treating, preventing, delaying, reducing or attenuating the development or risk of development of the adverse event is administered as one or more doses to the patient prior to the initiation of the treatment with the multispecific (e.g. bispecific) antibody as a prophylactic treatment for the adverse event.

In some embodiments, the agent capable of treating, preventing, delaying, reducing or attenuating the development or risk of development of the adverse event is administered to the patient in combination with one or more dose of the multispecific (e.g. bispecific) antibody as a prophylactic treatment for the adverse event. The agent may be administered as one or more doses consecutively (before and/or after), and/or concurrently with the multispecific (e.g. bispecific) antibody.

In some embodiments, the agent capable of treating, preventing, delaying, reducing or attenuating the development or risk of development of the adverse event is administered to the patient in combination with the first dose of the multispecific (e.g. bispecific) antibody as a prophylactic treatment for the adverse event. The agent may be administered as one or more doses consecutively (before and/or after), and/or concurrently with the multispecific (e.g. bispecific) antibody.

In some embodiments, the agent capable of treating, preventing, delaying, reducing or attenuating the development or risk of development of the adverse event is administered to the patient in combination with each increase in dose of the multispecific (e.g. bispecific) antibody as a prophylactic treatment for the adverse event. The agent may be administered as one or more doses consecutively (before and/or after), and/or concurrently with the multispecific (e.g. bispecific) antibody.

In some embodiments in which the treatment comprises the administration of a first maintenance dose and one or more additional maintenance dose(s) of the multispecific (e.g. bispecific) antibody to the patient, the maintenance doses are administered in a dosing regimen comprising:
(i) a starting phase, wherein the first maintenance dose, and optionally one or more additional maintenance dose(s) of the multispecific (e.g. bispecific) antibody are administered to the patient in combination with a prophylactic treatment, followed by
(ii) a maintenance phase, wherein one or more maintenance dose(s) of the multispecific (e.g. bispecific) antibody is administered to the patient,

wherein the prophylactic treatment comprises administration of an agent to the patient as one or more doses consecutively (before and/or after), and/or concurrently with the maintenance dose of the multispecific (e.g. bispecific) antibody, wherein the agent is capable of treating, preventing, delaying, reducing or attenuating the development or risk of development of cytokine-driven toxicities (e.g. CRS).

Administration of the multispecific (e.g. bispecific) antibody in combination with the prophylactic treatment in the starting phase significantly reduces toxicity due to attenuation of cytokine release. Thus, it will be appreciated that the maintenance doses of the starting phase may comprise high doses of the multispecific (e.g. bispecific) antibody as described herein, e.g. of about 8.5 mg to 11.5 mg, from about 9 mg to 11 mg, from about 9.5 mg to 10.5 mg, e.g. about 10 mg.

In preferred embodiments, the prophylactic treatment comprises administration of at least one dose of the agent (e.g. CRS agent) before the maintenance dose of the multispecific (e.g. bispecific) antibody. In some embodiments, the prophylactic treatment comprises administration of one or more doses (e.g. two doses) of the agent (e.g. CRS agent) before the maintenance dose and administered of one or more doses of the agent (e.g. CRS agent) after the maintenance dose.

In some embodiments of any aspect of the invention, the prophylactic treatment comprises administration of the agent (e.g. CRS agent) at an amount sufficient to prevent, delay, reduce or attenuate the development or risk of development of the adverse event (e.g. CRS).

In some embodiments of any aspect of the invention, the prophylactic treatment comprises the administration of a corticosteroid, such as dexamethasone. In some embodiments, the dexamethasone is administered at a dose of about 10-20 mg, preferably intravenously. In embodiments in which dexamethasone is administered as a prophylactic treatment for a cytokine-driven toxicity (e.g. CRS), preferably dexamethasone is administered at an amount sufficient to attenuate secretion of cytokines (e.g. GM-CSF, IL-2 and/or TNF-α) induced by the multispecific (e.g. bispecific) antibody of the invention.

In some embodiments of any aspect of the invention, the prophylactic treatment comprises the administration of an antagonist of a cytokine receptor or cytokine, such as an antagonist of IL-6, an IL-6 receptor (IL-6R), IL-1 (e.g. IL-1α, IL-1β, IL-1RA) and/or an IL-1 receptor (IL-1R) wherein the antagonist is selected from an antibody or antigen-binding fragment, a small molecule, a protein or peptide and a nucleic acid.

In some embodiments of any aspect of the invention, the prophylactic treatment comprises an anti-IL-6 antagonist antibody and/or an anti-IL-6R antagonist antibody, e.g. tocilizumab. In some embodiments, tocilizumab is administered to the patient as a one or more doses of about 8 mg/kg, preferably intravenously. In preferred embodiments, tocilizumab is administered at least 30 minutes prior to the multispecific (e.g. bispecific) antibody. In embodiments in which tocilizumab is administered as a prophylactic treatment for a cytokine-driven toxicity (e.g. CRS), preferably tocilizumab is administered at an amount sufficient to attenuate IL-6 receptor signalling induced by the multispecific (e.g. bispecific) antibody of the invention.

In certain embodiments, the starting phase comprises a first and second maintenance dose of the multispecific (e.g. bispecific) antibody each administered to the patient in combination with a prophylactic treatment, wherein the prophylactic treatment comprises a single dose of tocilizumab administered at least 30 minutes prior to the maintenance dose, optionally wherein the second maintenance dose is administered to the patient 7 days after the first maintenance dose.

In some embodiments of any aspect of the invention, the prophylactic treatment comprises an anti-IL-1 antagonist and/or an anti-IL-1R antagonist, e.g. anakinra. In some embodiments, anakinra is administered as a prophylactic treatment for a cytokine-driven toxicity (e.g. CRS), preferably at an amount sufficient to attenuate IL-1 receptor signalling induced by the multispecific (e.g. bispecific) antibody of the invention. Anakinra may be administered at a dose of about 100 mg (e.g. 100 mg±20%), preferably subcutaneously. In some embodiments, anakinra is administered to the patient as a dose of about 100 mg, preferably subcutaneously. In some embodiments, the prophylactic treatment comprises at least one dose of anakinra administered before the multispecific (e.g. bispecific) antibody, and at least one dose of anakinra administered after the multispecific (e.g. bispecific) antibody.

Anakinra may be administered to the patient as one or more fixed dose(s) between about 16 hours to about 2 hours prior to the multispecific (e.g. bispecific) antibody, and optionally a fixed dose between about 20 hours to about 22 hours after the multispecific (e.g. bispecific) antibody. In some embodiments of any aspect of the invention, anakinra is administered as:
(i) a fixed dose between about 16 hours to about 8 hours prior to the multispecific (e.g. bispecific) antibody; and/or
(ii) a fixed dose between about 4 hours to about 2 hours prior to the multispecific (e.g. bispecific) antibody,

optionally wherein an additional fixed dose of anakinra is administered between about 20 hours to about 22 hours after the multispecific (e.g. bispecific) antibody.

In certain embodiments, the starting phase comprises a first and second maintenance dose of the multispecific (e.g. bispecific) antibody, each administered to the patient in combination with a prophylactic treatment, wherein
(i) the first maintenance dose is administered in combination with: a first dose of anakinra between about 16 hours to about 8 hours prior to the maintenance dose; a second dose of anakinra between about 4 hours to about 2 hours prior to the maintenance dose; and a third dose of anakinra between about 20 hours to about 22 hours after the maintenance dose; and
(ii) the second maintenance dose is administered in combination with: a fourth dose of anakinra between about 4 hours to about 2 hours prior to the maintenance dose; and a fifth dose of anakinra between about 20 hours to about 22 hours after the maintenance dose,

optionally wherein the second maintenance dose is administered to the patient 7 days after the first maintenance dose.

In some embodiments of any aspect of the invention, the prophylactic treatment comprises the administration of dexamethasone (e.g. about 10-20 mg, preferably intravenously) with tocilizumab (e.g. about 8 mg/kg, preferably intravenously). In some embodiments, the prophylactic treatment comprises the administration of dexamethasone (e.g. about 10-20 mg, preferably intravenously) with anakinra (e.g. about 100 mg, preferably subcutaneously).

In some embodiments of any aspect of the invention, the prophylactic treatment comprises the administration of symptomatic support, including administration of antipyretics, analgesics, antivirals and/or antibiotics. In some embodiments, the symptomatic support comprises the administration of antivirals (e.g. acyclovir, oseltamivir, zanamivir and/or equivalents) and/or antibiotics (e.g. trimethoprim-sulfamethoxazole, levofloxacin and/or equivalents). In some embodiments, the prophylactic treatment comprises the administration of seizure prophylaxis (e.g. levetiracetam). The symptomatic support and/or seizure prophylaxis may be administered in addition to the agent capable of treating, preventing, delaying, reducing or attenuating the development or risk of development of the adverse event.

In some embodiments of any aspect of the invention, the agent capable of treating, preventing, delaying, reducing or attenuating the development or risk of development of the adverse event is administered to the patient in the event that the patient develops an adverse event associated with the administration of the multispecific (e.g. bispecific) antibody. In some embodiments, the treatment comprises administration of the agent at a therapeutic amount, or an amount sufficient to partially or completely alleviate or ameliorate the adverse event (e.g. CRS) or symptoms thereof.

If the patient develops an adverse event (e.g. CRS) following administration of a multispecific (e.g. bispecific) antibody of the invention, the treatment may further comprise the administration of an anti-IL-6R antagonist antibody, e.g., tocilizumab. In some embodiments, tocilizumab is administered to the patient as a single dose of about 8 mg/kg, preferably intravenously. In some embodiments, the treatment may further include administering to the patient one or more additional doses of an IL-6R antagonist antibody, e.g., tocilizumab. In some embodiments, tocilizumab is administered to the patient in one or more additional doses of about 8 mg/kg, preferably intravenously.

In some embodiments, if the patient develops an adverse event (e.g. CRS) following administration of a multispecific (e.g. bispecific) antibody of the invention, the treatment may further comprise the administration of an anti-IL-1 antagonist and/or an anti-IL-1R antagonist, e.g. anakinra. In some embodiments, anakinra is administered to the patient as one more fixed doses of about 100 mg, preferably subcutaneously. In some embodiments, anakinra is administered to the patient twice daily, preferably as fixed doses of about 100 mg, preferably subcutaneously.

In some embodiments, if an adverse event (e.g. CRS) occurs the treatment may further comprise the administration of an IL-6 antagonist antibody, e.g., siltuximab. In some embodiments, siltuximab is administered to the patient as a single dose of about 11 mg/kg, preferably intravenously

In some embodiments, if an adverse event (e.g. CRS) occurs the treatment may further comprise administering to the patient a corticosteroid, such as methylprednisolone or dexamethasone. In some embodiments, the dexamethasone is administered at a dose of about 10-20 mg, preferably intravenously. In some embodiments, the methylprednisolone is administered at a dose of about 1 mg/kg per day to about 5 mg/kg per day, e.g., about 2 mg/kg per day.

In some embodiments, the additional treatments may be based on the stage of the CRS. A modification of the common CTCAE CRS grading scale has been established for the grading and treatment of CRS, and is detailed in Table 10:

TABLE 10
Grading and Treatment of Cytokine Release Syndrome

| | | | | | CRS Grade 4 |
|---|---|---|---|---|---|
| | Symptoms/ | CRS Grade 1 | CRS Grade 2 | CRS Grade 3 | (Life- |
| | Signs | (Mild) | (Moderate) | (Severe) | Threatening) |
| Vital Signs | Temp ≥ 38° C. | Yes | Any | Any | Any |
| | Systolic blood | No | Responds to | Needs high | Life- |
| | pressure | | intravenous | dose | threatening |
| | (SBP) ≤ 90 | | (IV) | or | |
| | mm Hg | | fluids or single | multiple | |
| | | | low-dose | vasopressors | |
| | | | vasopressor | | |
| | Need for | No | FiO2 < 40% | FiO2 ≥ 40% | Needs |
| | oxygen | | | | ventilator |
| | for O2 | | | | support |
| | sat > 90% | | | | |
| Organ | | Grade 1 | Grade 2 | Grade 3 or | Grade 4 |
| Toxicity | | | | transaminitis | |
| | | | | Grade 4 | |

For example, in embodiments in which the patient has a grade 2 CRS following administration of the multispecific (e.g. bispecific) antibody, the treatment may further comprise the administration of a first line treatment comprising the administration of a first dose of an anti-IL-6 antagonist antibody and/or an anti-IL-6R antagonist antibody, e.g., tocilizumab. In some instances, tocilizumab is administered intravenously to the patient as a single dose of about 8 mg/kg.

In alternative embodiments in which the patient has a grade 2 CRS following administration of the multispecific (e.g. bispecific) antibody, the treatment may further comprise the administration of a first line treatment comprising the administration of one or more fixed dose(s) of anti-IL-1 antagonist and/or an anti-IL-1R antagonist, e.g. anakinra. Anakinra may be administered at a dose of about 100 mg (e.g. 100 mg±20%), preferably subcutaneously. In some embodiments, anakinra is administered to the patient as one more fixed dose(s) of about 100 mg, preferably subcutaneously. In some embodiments, anakinra is administered to the patient twice daily, preferably as fixed doses of about 100 mg, preferably subcutaneously.

If the patient develops rapid onset of grade 2 CRS or develops grade ≥3 CSR onset following administration of the multispecific (e.g. bispecific) antibody, the treatment may further comprise the administration of a first line treatment comprising:
(i) an anti-IL-6 antagonist antibody and/or an anti-IL-6R antagonist antibody, e.g., tocilizumab; and
(ii) a corticosteroid, e.g. dexamethasone or methylprednisolone.

In some embodiments, tocilizumab is administered intravenously to the patient at a dose of about 8 mg/kg.

Alternatively, if the patient develops rapid onset of grade 2 CRS or develops grade ≥3 CRS onset following administration of the multispecific (e.g. bispecific) antibody, the treatment may further comprise the administration of a first line treatment comprising:
(i) an anti-IL-1 antagonist and/or an anti-IL-1R antagonist, e.g. anakinra; and
(ii) a corticosteroid, e.g. dexamethasone or methylprednisolone.

In some embodiments, anakinra is administered to the patient as one more fixed dose(s) of about 100 mg, preferably subcutaneously. In some embodiments, anakinra is administered to the patient twice daily, preferably as fixed doses of about 100 mg, preferably subcutaneously.

The corticosteroid may be administered consecutively (before or after) or concurrently with the (i) anti-IL-6 antagonist antibody and/or an anti-IL-6R antagonist antibody, e.g., tocilizumab, or (ii) anti-IL-1 antagonist and/or an anti-IL-1R antagonist, e.g. anakinra. In some embodiments, the corticosteroid is dexamethasone. In some embodiments, the dexamethasone is administered at a dose of about 10-20 mg, preferably intravenously. In some embodiments, the corticosteroid is methylprednisolone. In some embodiments, methylprednisolone is administered at a dose of about 1 mg/kg per day to about 5 mg/kg per day, e.g., about 2 mg/kg per day.

In some embodiments, the first line treatment comprises the administration of symptomatic support for CRS, including administration of antipyretics, analgesics and/or antibiotics. In some embodiments, the first line treatment comprises the administration of seizure prophylaxis (e.g. levetiracetam). The symptomatic support and/or seizure prophylaxis may be administered in addition to the agent capable of treating, preventing, delaying, reducing or attenuating the development or risk of development of the adverse event.

In some embodiments of any aspect of the invention, if the patient develops CRS following administration of a dose of the multispecific (e.g. bispecific) antibody (e.g. starting dose or maintenance dose), the next dose (e.g. next starting dose or next maintenance dose) may be administered to the patient when toxicity reaches Grade SI as described herein. In alternative embodiments where the patient develops CRS, the next dose may be administered to the patient when toxicity reaches baseline levels.

In the event that the CRS does not resolve or worsens in response to first line treatment, the treatment may further comprise the administration of a second line treatment comprising:
(i) one or more (e.g., one, two, three, four, or five or more) additional doses of the anti-IL-6 antagonist antibody and/or an anti-IL-6R antagonist antibody, e.g., tocilizumab; and
(ii) one or more (e.g., one, two, three, four, or five or more) additional doses of the corticosteroid, e.g. dexamethasone or methylprednisolone.

In some embodiments, the one or more additional doses of tocilizumab are administered intravenously to the patient at a dose of about 8 mg/kg. The corticosteroid may be administered consecutively (before or after) or concurrently with the anti-IL-6 antagonist antibody and/or an anti-IL-6R antagonist antibody, e.g., tocilizumab. In some embodiments, the corticosteroid is dexamethasone. In some embodiments, the dexamethasone is administered at a dose of about 10-20 mg, preferably intravenously. In some embodiments, the corticosteroid is methylprednisolone. In some embodiments, methylprednisolone is administered at a dose of about 1 mg/kg per day to about 5 mg/kg per day, e.g., about 2 mg/kg per day.

In the event that the CRS does not resolve or worsens in response to second line treatment, the treatment may further comprise the administration of a third line treatment comprising the administration of an antagonist of a cytokine receptor or cytokine selected from among GM-CSF, IL-10, IL-10R, IL-6, IL-6 receptor (IL-6R), IFNγ, IFNGR, IL-2, IL-2R/CD25, MCP-1, CCR2, CCR4, MIPIβ, CCR5, TNFalpha, TNFR1, IL-1 (e.g. IL-1α, IL-10, IL-1RA), and IL-1 receptor (IL-1R), wherein the antagonist is selected from an antibody or antigen-binding fragment, a small molecule, a protein or peptide and a nucleic acid. The antagonist may be an anti-IL-6 antibody and/or an anti-IL6R antibody. For example, the antagonist may be selected from tocilizumab, siltuximab, clazakizumab, sarilumab, olokizumab, elsilimomab, ALD518/BMS-945429, sirukumab (CNTO 136), CPSI-2634, ARGX-109, lenzilumab, FE301 and FM101. In some embodiments, the third line treatment comprises the administration of siltuximab. In some embodiments, siltuximab is administered to the patient as a single dose of about 11 mg/kg, preferably intravenously. Alternatively, the antagonist may be an anti-IL-1 antagonist and/or an anti-IL-1R antagonist e.g. anakinra.

In the event that the CRS does not resolve or worsens in response to third line treatment, the treatment may further comprise the administration of a fourth line treatment comprising the administration of a molecule that decreases the regulatory T cell (Treg) population. Molecules that decrease the number of (e.g., deplete) Treg cells are known in the art and include, e.g., CD25 depletion, cyclophosphamide administration, anti-CTLA4 antibody and modulating Glucocorticoid-induced TNLR family related gene (GITR) function. GITR is a member of the TNLR superfamily that is upregulated on activated T cells, which enhances the immune system. In some embodiments, the fourth line treatment comprises the administration of cyclophosphamide.

In some embodiments, if an adverse event occurs (e.g. neutropenia, infection) the treatment may further comprise symptomatic support, including administration of antipyretics, analgesics, antivirals and/or antibiotics. In some embodiments, seizure prophylaxis (e.g. levetiracetam) can be administered to the patient. If the patient develops neutropenia (e.g. at least grade 3 neutropenia), the treatment may further comprise administration of antibiotics (e.g. levofloxacin or equivalent). If the patient develops a viral infection (e.g. influenza), the treatment may further comprise administration of oseltamivir, zanamivir and/or equivalents.

In some embodiments of any aspect of the invention, if the patient develops a viral infection (e.g. influenza A/B, SARS-CoV-2) following administration of a dose of the multispecific (e.g. bispecific) antibody (e.g. starting dose or maintenance dose), the next dose (e.g. next starting dose or next maintenance dose) may be administered to the patient when symptoms of the infection resolve. In alternative embodiments where the patient develops a viral infection, the next dose may be administered after a negative test for the viral infection, e.g. a negative PCR viral panel, and/or at least 14 days after a positive test for the viral infection, e.g. a positive PCR viral panel. A viral panel (e.g. PCR viral panel) may test for influenza A/B, respiratory syncytial virus, parainfluenza virus, metapneumovirus, adenovirus and/or SARS-CoV-2.

The Multispecific Antibody

The multispecific (e.g. bispecific) antibodies of the invention specifically bind to BCMA and to CD3. The terms “antibody against BCMA and CD3”, “anti-BCMA anti-CD3 antibody” or “an antibody that binds to BCMA and CD3,” refer to a multispecific antibody (e.g., a bispecific antibody) that is capable of binding to BCMA and CD3 with sufficient affinity such that the antibody is useful as a therapeutic agent. This is achieved by making a molecule which comprises a first antibody, or antigen-binding fragment, that binds to BCMA and a second antibody, or antigen-binding fragment, that binds to CD3. Such multispecific antibodies may be trispecific antibodies or bispecific antibodies. In preferred embodiments, the multispecific antibodies are bispecific antibodies.

The term “BCMA” as used herein relate to human B cell maturation antigen, also known as BCMA; TR17_HUMAN, TNFRSF17 (UniProt Q02223), which is a member of the tumor necrosis receptor superfamily that is preferentially expressed in differentiated plasma cells. The extracellular domain of BCMA consists according to UniProt of amino acids 1-54 (or 5-51). The terms “antibody against BCMA”, “anti BCMA antibody” or “an antibody that binds to BCMA” as used herein relate to an antibody specifically binding to the extracellular domain of BCMA.

The term “specifically binding to BCMA” refers to an antibody that is capable of binding to the defined target with sufficient affinity such that the antibody is useful as a therapeutic agent in targeting BCMA. In some embodiments, an antibody specifically binding to BCMA does not bind to other antigens, or does not bind to other antigens with sufficient affinity to produce a physiological effect.

In some embodiments, the extent of binding of an anti-BCMA antibody to an unrelated, non-BCMA protein is about 10-fold preferably >100-fold less than the binding of the antibody to BCMA as measured, e.g., by surface plasmon resonance (SPR) e.g. Biacore®, enzyme-linked immunosorbent (ELISA) or flow cytometry (FACS). In one embodiment the antibody that binds to BCMA has a dissociation constant (Kd) of 10⁻⁸ M or less, preferably from 10⁻⁸ M to 10⁻¹³ M, preferably from 10⁻⁹ M to 10⁻¹³ M.

In one embodiment the anti-BCMA antibody binds to an epitope of BCMA that is conserved among BCMA from different species, preferably among human and cynomolgus, and in addition preferably also to mouse and rat BCMA.

Preferably the anti-BCMA antibody specifically binds to a group of BCMA, consisting of human BCMA and BCMA of non-human mammalian origin, preferably BCMA from cynomolgus, mouse and/or rat. Anti-BCMA antibodies are analyzed by ELISA for binding to human BCMA using plate-bound BCMA. For this assay, an amount of plate-bound BCMA preferably 1.5 μg/mL and concentration(s) ranging from 0.1 pM to 200 nM of anti-BCMA antibody are used.

The term “CD3” refers to the human CD3 protein multi-subunit complex. The CD3 protein multi-subunit complex is composed to 6 distinctive polypeptide chains. Thus the term includes a CD3γ chain (SwissProt P09693), a CD36 chain (SwissProt P04234), two CD3ε chains (SwissProt P07766), and one CD3ζ chain homodimer (SwissProt 20963), and which is associated with the T cell receptor α and β chain. The term encompasses “full-length,” unprocessed CD3, as well as any CD3 variant, isoform and species homolog which is naturally expressed by cells (including T cells) or can be expressed on cells transfected with genes or cDNA encoding those polypeptides.

The term “specifically binding to CD3” refers to an antibody that is capable of binding to the defined target with sufficient affinity such that the antibody is useful as a therapeutic agent in targeting CD3. In some embodiments, an antibody specifically binding to CD3 does not bind to other antigens, or does not bind to other antigens with sufficient affinity to produce a physiological effect.

The multispecific (e.g. bispecific) antibodies of the invention can be analysed by SPR, e.g. Biacore®, for binding to CD3. In some embodiments, the bispecific antibodies bind to human CD3 with a dissociation constant (K_{D}) of about 10⁻⁷ M or less, a K_{D} of about 10⁻⁸ M or less, a K_{D} of about 10⁻⁹ M or less, a K_{D} of about 10⁻¹⁰ M or less, a K_{D} of about 10⁻¹¹ M or less, or a K_{D} of about 10⁻¹² M or less, as determined by a surface plasmon resonance assay, preferably measured using Biacore 8K at 25° C. In preferred embodiments, the bispecific antibodies bind to human CD3 with a dissociation constant (K_{D}) of about 10⁻⁸ M or less.

The term “antibody” herein encompasses various antibody structures, including but not limited to monoclonal antibodies, polyclonal antibodies, multispecific antibodies (e.g., bispecific antibodies), and antibody fragments so long as they exhibit the desired antigen-binding activity.

A “heavy chain” comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM).

A “light chain” comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies according to the invention and therefore provide a further aspect of the invention.

The term “antigen binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen. Thus, the term refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The terms “Fab fragment” and “Fab” are used interchangeably herein and contain a single light chain (i.e. a constant domain CL and a VL) and a single heavy chain (i.e. the constant domain CH1 and a VH). The heavy chain of a Fab fragment is not capable of forming a disulfide bond with another heavy chain.

A “Fab′ fragment” contains a single light chain and a single heavy chain but in addition to the CH1 and the VH, a “Fab′ fragment” contains the region of the heavy chain between the CH1 and CH2 domains that is required for the formation of an inter-chain disulfide bond. Thus, two “Fab′ fragments” can associate via the formation of a disulphide bond to form a F(ab′)2 molecule.

A “F(ab′)2 fragment” contains two light chains and two heavy chains. Each chain includes a portion of the constant region necessary for the formation of an inter-chain disulfide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “bi-specific T cell engager” (BiTE®) is a fusion protein consisting of two single-chain variable fragments (scFvs) on a single peptide chain. One of the scFvs binds to T cells via the CD3 receptor, and the other to a tumour cell antigen.

A “diabody” is a small bivalent and bispecific antibody fragment comprising a heavy (VH) chain variable domain connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

A “DARPin” is a bispecific ankyrin repeat molecule. DARPins are derived from natural ankyrin proteins, which can be found in the human genome and are one of the most abundant types of binding proteins. A DARPin library module is defined by natural ankyrin repeat protein sequences, using 229 ankyrin repeats for the initial design and another 2200 for subsequent refinement. The modules serve as building blocks for the DARPin libraries. The library modules resemble human genome sequences. A DARPin is composed of 4 to 6 modules. Because each module is approx. 3.5 kDa, the size of an average DARPin is 16-21 kDa. Selection of binders is done by ribosome display, which is completely cell-free and is described in He M. and Taussig M J., Biochem Soc Trans. 2007, November; 35(Pt 5):962-5.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, Md. (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)).

TABLE 11
CDR definitions

| | Kabat | Chothia | IMGT |
|---|---|---|---|
| VH CDR1 | 31-35 | 26-32 | 27-38 |
| VH CDR2 | 50-65 | 52-56 | 56-65 |
| VH CDR3 | 95-102 | 95-102 | 105-117 |
| VL CDR1 | 24-34 | 24-34 | 27-38 |
| VL CDR2 | 50-56 | 50-56 | 56-65 |
| VL CDR3 | 89-97 | 89-97 | 105-117 |

For heavy chain constant region amino acid positions discussed in the invention, numbering is according to the EU index first described in Edelman, G. M., et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”. “EU index of Kabat” or “EU numbering” in the context of the heavy chain refers to the residue numbering system based on the human igG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat set forth in Kabat et al. (supra.).

The antibodies of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

Especially preferred are human or humanized antibodies, especially as recombinant human or humanized antibodies.

The term “humanized antibody” refers to antibodies in which the framework or “complementarity determining regions” (CDRs) have been modified to comprise the CDR of an immunoglobulin of different specificity as compared to that of the parent immunoglobulin. For example, a murine CDR may be grafted into the framework region of a human antibody to prepare the “humanized antibody.” See, e.g., Riechmann, L., et al., Nature 332 (1988) 323-327; and Neuberger, M. S., et al., Nature 314 (1985) 268-270. In some embodiments, “humanized antibodies” are those in which the constant region has been additionally modified or changed from that of the original antibody to generate the properties of the antibodies according to the invention, especially in regard to C1q binding and/or Fc receptor (FcR) binding.

The term “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties of the antibodies according to the invention, especially in regard to C1q binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244.

The terms “Fc region” and “Fc” are used interchangeably herein and refer to the portion of a native immunoglobulin that is formed by two Fc chains. Each “Fc chain” comprises a constant domain CH2 and a constant domain CH3. Each Fe chain may also comprise a hinge region. A native Fc region is homodimeric. In some embodiments, the Fc region may contain modifications to enforce Fc heterodimerization.

The term “Fc part” refers to the portion of an antibody of the invention, or antigen binding fragment thereof, which corresponds to the Fc region.

There are five major classes of heavy chain constant region, classified as IgA, IgG, IgD, IgE and IgM, each with characteristic effector functions designated by isotype. For example, IgG is separated into four subclasses known as IgG1, IgG2, IgG3, and IgG4. Ig molecules interact with multiple classes of cellular receptors. For example, IgG molecules interact with three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains.

The antibodies of the invention or antigen-binding fragments thereof may be any isotype, i.e. IgA, IgD, IgE, IgG and IgM, and synthetic multimers of the four-chain immunoglobulin (Ig) structure. In preferred embodiments, the antibodies or antigen-binding fragments thereof are IgG isotype. The antibodies or antigen-binding fragments can be any IgG subclass, for example IgG1, IgG2, IgG3, or IgG4 isotype. In preferred embodiments, the antibodies or antigen-binding fragments thereof are of an IgG1 isotype.

In some embodiments, the antibodies comprise a heavy chain constant region that is of IgG isotype. In some embodiments, the antibodies comprise a portion of a heavy chain constant region that is of IgG isotype. In some embodiments, the IgG constant region or portion thereof is an IgG1, IgG2, IgG3, or IgG4 constant region. Preferably, the IgG constant region or portion thereof is an IgG1 constant region.

The antibodies of the invention or antigen-binding fragments thereof may comprise a lambda light chain or a kappa light chain.

In preferred embodiments, the antibodies or antigen-binding fragments thereof comprise a light chain that is a kappa light chain. In some embodiments, the antibody or antigen-binding fragment comprises a light chain comprising a light chain constant region (CL) that is a kappa constant region.

In some embodiments, the antibody comprises a light chain comprising a light chain variable region (VL) that is a kappa variable region. Preferably, the kappa light chain comprises a VL that is a kappa VL and a CL that is a kappa CL.

Alternatively, the antibodies or antigen-binding fragments thereof may comprise a light chain that is a lambda light chain. In some embodiments, the antibody or antigen-binding fragment comprises a light chain comprising a light chain constant region (CL) that is a lambda constant region. In some embodiments, the antibody comprises a light chain comprising a light chain variable region (VL) that is a lambda variable region.

Engineered antibodies and antigen-binding fragments thereof include those in which modifications have been made to framework residues within the VH and/or VL. Such modifications may improve the properties of the antibody, for example to decrease the immunogenicity of the antibody and/or improve antibody production and purification.

Antibodies and antigen-binding fragments thereof disclosed herein can be further modified using conventional techniques known in the art, for example, by using amino acid deletion(s), insertion(s), substitution(s), addition(s), and/or recombination(s) and/or any other modification(s) known in the art, either alone or in combination. Methods for introducing such modifications in the DNA sequence underlying the amino acid sequence of an immunoglobulin chain arc well known to the person skilled in the art.

The antibodies of the invention and antigen-binding fragments thereof also include derivatives that are modified (e.g., by the covalent attachment of any type of molecule to the antibody) such that covalent attachment does not prevent the antibody from binding to its epitope, or otherwise impair the biological activity of the antibody. Examples of suitable derivatives include, but are not limited to fucosylated antibodies, glycosylated antibodies, acetylated antibodies, PEGylated antibodies, phosphorylated antibodies, and amidated antibodies.

Minor variations in the amino acid sequences of antibodies of the invention are contemplated as being encompassed by the present invention, providing that the variations in the amino acid sequence(s) maintain at least 75%, more preferably at least 80%, at least 90%, at least 95%, and most preferably at least 99% sequence identity to the antibody of the invention or antigen-binding fragment thereof as defined anywhere herein.

Antibodies of the invention may include variants in which amino acid residues from one species are substituted for the corresponding residue in another species, either at the conserved or non-conserved positions. In one embodiment, amino acid residues at non-conserved positions are substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in an antibody of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Antibody Format

Formats for multispecific, e.g. bispecific, antibodies are known in the state of the art. For example, bispecific antibody formats are described in Kontermann R E, mAbs 4:2 1-16 (2012); Holliger P., Hudson P J, Nature Biotech. 23 (2005) 1126-1136, Chan A C, Carter P J Nature Reviews Immunology 10, 301-316 (2010) and Cuesta A M et al., Trends Biotech 28 (2011) 355-362.

The multispecific, e.g. bispecific, antibodies of the invention may have any format. Multispecific and bispecific antibody formats include, for example, multivalent single chain antibodies, diabodies and triabodies, and antibodies having the constant domain structure of full length antibodies to which further antigen-binding domains (e.g., single chain Fv, a tandem scFv, a VH domain and/or a VL domain, Fab, or (Fab)₂) are linked via one or more peptide-linkers, as well as antibody mimetics such as DARPins. In some embodiments, the multispecific, e.g. bispecific, antibodies of the invention have the format of an scFv such as a bispecific T cell engager (BITE®). In some embodiments, the antibodies of the invention are single chain antibodies which comprise a first domain which binds to BCMA, a second domain which binds to a T cell antigen (e.g. CD3), and a third domain which comprises two polypeptide monomers, each comprising a hinge, a CH2 domain and a CH3 domain, wherein the two polypeptide monomers are fused to each other via a peptide linker (e.g. (hinge-CH2-CH3-linker-hinge-CH2-CH3).

The “valency” of an antibody denotes the number of binding domains. As such, the terms “bivalent”, “trivalent”, and “multivalent” denote the presence of two binding domains, three binding domains, and multiple binding domains, respectively. The multispecific, e.g. bispecific, antibodies of the invention may have more than one binding domain capable of binding to each target antigen (i.e., the antibody is trivalent or multivalent). In preferred embodiments, the multispecific, e.g. bispecific, antibodies of the invention have more than one binding domain capable of binding to the same epitope of each target antigen. In some embodiments, the multispecific, e.g. bispecific, antibodies of the invention have more than one binding domain capable of binding to different epitopes on each target antigen.

The multispecific, e.g. bispecific, antibodies of the invention may be bivalent, trivalent or tetravalent. In preferred embodiments, the multispecific, e.g. bispecific, antibody is trivalent, preferably wherein the trivalent antibody is bivalent for BCMA. Thus, the bispecific antibody may be trivalent, wherein the trivalent antibody is bivalent for BCMA.

The multispecific, e.g. bispecific, antibodies can be full length from a single species, or can be chimerized or humanized. For an antibody with more than two antigen-binding domains, some binding domains may be identical, as long as the protein has binding domains for two different antigens.

The multispecific, e.g. bispecific, antibodies of the invention can have a bispecific heterodimeric format. In some embodiments, the bispecific antibody comprises two different heavy chains and two different light chains. In other embodiments, the multispecific, e.g. bispecific, antibody comprises two identical light chains and two different heavy chains. In some embodiments, in the multispecific, e.g. bispecific, antibodies of the invention one of the two pairs of heavy chain and light chain (HC/LC) specifically binds to CD3 and the other one specifically binds to BCMA.

In embodiments in which the bispecific antibodies of the invention are bivalent, they may comprise one anti-BCMA antibody and one anti-CD3 antibody (referred to herein as the “1+1” format).

In embodiments in which the BCMA and CD3 antibodies are Fabs, the bivalent bispecific antibodies in the 1+1 format may have the format: CD3 Fab-BCMA Fab (i.e. when no Fc is present). Alternatively, the bispecific antibodies may have the format: Fc-CD3 Fab-BCMA Fab; Fc-BCMA Fab-CD3 Fab; or BCMA Fab-Fc-CD3 Fab (i.e. when an Fc is present). In preferred embodiments, the bivalent bispecific antibodies have the format BCMA Fab-Fe-CD3 Fab.

“CD3 Fab-BCMA Fab” means that the CD3 Fab is bound via its N-terminus to the C-terminus of the BCMA Fab.

“Fc-BCMA Fab-CD3 Fab” means that the BCMA Fab is bound via its C-terminus to the N-terminus of the Fc, and the CD3 Fab is bound via its C-terminus to the N-terminus of the BCMA Fab.

“Fc-CD3 Fab-BCMA Fab” means that the CD3 Fab is bound via its C-terminus to the N-terminus of the Fc, and the BCMA Fab is bound via its C-terminus to the N-terminus of the CD3 Fab.

“BCMA Fab-Fc-CD3 Fab” means that the BCMA and CD3 Fab fragments are bound via their C-terminus to the N-terminus of the Fc.

In embodiments in which the bispecific antibodies of the invention are trivalent, they may comprise two anti-BCMA antibodies and one anti-CD3 antibody (referred to herein as the “2+1” format).

In embodiments in which the BCMA and CD3 antibodies are Fabs, the trivalent bispecific antibodies in the 2+1 format may have the format: CD3 Fab-BCMA Fab-BCMA Fab; or BCMA Fab-CD3 Fab-BCMA Fab (i.e. when no Fc is present). Alternatively, the bispecific antibodies may have the format: BCMA Fab-Fc-CD3 Fab-BCMA Fab; BCMA Fab-Fc-BCMA Fab-CD3 Fab; or CD3 Fab-Fc-BCMA Fab-BCMA Fab (i.e. when an Fc is present). In preferred embodiments, the trivalent bispecific antibodies have the format BCMA Fab-Fc-CD3 Fab-BCMA Fab.

“CD3 Fab-BCMA Fab-BCMA Fab” means that the CD3 Fab is bound via its C-terminus to the N-terminus of the first BCMA Fab, and the first BCMA Fab is bound via its C-terminus to the N-terminus of the second BCMA Fab.

“BCMA Fab-CD3 Fab-BCMA Fab” means that the first BCMA Fab is bound via its C-terminus to the N-terminus of the CD3 Fab, and the CD3 Fab is bound via its C-terminus to the N-terminus of the second BCMA Fab.

“BCMA Fab-Fc-CD3 Fab-BCMA Fab” means that the first BCMA Fab and the CD3 Fab are bound via their C-terminus to the N-terminus of the Fc, and the second BCMA Fab is bound via its C-terminus to the N-terminus of the CD3 Fab.

“BCMA Fab-Fc-BCMA Fab-CD3 Fab” means that the first BCMA Fab and the second BCMA Fab are bound via their C-terminus to the N-terminus of the Fc, and the CD3 Fab is bound via its C-terminus to the N-terminus of the second BCMA Fab.

“CD3 Fab-Fc-BCMA Fab-BCMA Fab” means that the CD3 Fab and the first BCMA Fab are bound via their C-terminus to the N-terminus of the Fc, and the second BCMA Fab is bound via its C-terminus to the N-terminus of the first BCMA Fab.

In some embodiments, the bispecific antibodies of the invention may comprise not more than one BCMA Fab specifically binding to BCMA, and not more than one CD3 Fab specifically binding to CD3 and not more than one Fc part.

In some embodiments, the bispecific antibody comprises not more than one CD3 Fab specifically binding to CD3, not more than two BCMA Fabs specifically binding to BCMA and not more than one Fc part. In some embodiments, not more than one CD3 Fab and not more than one BCMA Fab are linked to the Fc part and linking is performed via C-terminal binding of the Fab(s) to the hinge region of the Fc part. In some embodiments, the second BCMA Fab is linked via its C-terminus either to the N-terminus of the CD3 Fab or to the hinge region of the Fc part and is therefore between the Fc part of the bispecific antibody and the CD3 Fab.

In embodiments comprising two BCMA Fabs, the BCMA Fabs are preferably derived from the same antibody and are preferably identical in the CDR sequences, variable domain sequences VH and VL and/or the constant domain sequences CH1 and CL. Preferably, the amino acid sequences of the two BCMA Fab are identical.

The bispecific antibodies of the invention can also comprise scFvs instead of the Fabs. Thus, in some embodiments, the bispecific antibodies have any one of the above formats, wherein each Fab is replaced with a corresponding scFv.

The components, e.g. the Fab fragments, of the bispecific antibodies of the invention may be chemically linked together by the use of an appropriate linker according to the state of the art. In preferred embodiments, a (Gly4-Serl)₂ linker is used (Desplancq D K et al., Protein Eng. 1994 August; 7(8):1027-33 and Mack M. et al., PNAS Jul. 18, 1995 vol. 92 no. 15 7021-7025). “Chemically linked” (or “linked”) as used herein means that the components are linked by covalent binding. As the linker is a peptidic linker, such covalent binding is usually performed by biochemical recombinant means. For example, the binding may be performed using a nucleic acid encoding the VL and/or VH domains of the respective Fab fragments, the linker and the Fc part chain if the antibody comprises an Fc.

In the event that a linker is used, this linker may be of a length and sequence sufficient to ensure that each of the first and second domains can, independently from each other, retain their differential binding specificities.

Antibody Sequences

In some embodiments, the multispecific (e.g. bispecific) antibody comprises an anti-BCMA antibody, or antigen binding fragment thereof, comprising a CDR3H region of SEQ ID NO: 17 and a CDR3L region of SEQ ID NO:20 and a CDR1H, CDR2H, CDR1L, and CDR2L region combination selected from the group of
a) CDR1H region of SEQ ID NO:21 and CDR2H region of SEQ ID NO:22, CDR1L region of SEQ ID NO:23, and CDR2L region of SEQ ID NO:24,
b) CDR1H region of SEQ ID NO:21 and CDR2H region of SEQ ID NO:22, CDR1L region of SEQ ID NO:25, and CDR2L region of SEQ ID NO:26,
c) CDR1H region of SEQ ID NO:21 and CDR2H region of SEQ ID NO:22, CDR1L region of SEQ ID NO:27, and CDR2L region of SEQ ID NO:28,
d) CDR1H region of SEQ ID NO:29 and CDR2H region of SEQ ID NO:30, CDR1L region of SEQ ID NO:31, and CDR2L region of SEQ ID NO:32,
e) CDR1H region of SEQ ID NO:34 and CDR2H region of SEQ ID NO:35, CDR1L region of SEQ ID NO:31, and CDR2L region of SEQ ID NO:32,
f) CDR1H region of SEQ ID NO:36 and CDR2H region of SEQ ID NO:37, CDR1L region of SEQ ID NO:31, and CDR2L region of SEQ ID NO:32, and
g) CDR1H region of SEQ ID NO: 15 and CDR2H region of SEQ ID NO: 16, CDR1L region of SEQ ID NO:18, and CDR2L region of SEQ ID NO:19.

In preferred embodiments, the multispecific (e.g. bispecific) antibody comprises an anti-BCMA antibody, or antigen binding fragment thereof, comprising a VH region comprising a CDR1H region of SEQ ID NO:21, a CDR2H region of SEQ ID NO:22 and a CDR3H region of SEQ ID NO:17 and a VL region comprising a CDR3L region of SEQ ID NO:20 and a CDR1L and CDR2L region combination selected from the group of:
i) CDR1L region of SEQ ID NO:27 and CDR2L region of SEQ ID NO:28;
ii) CDR1L region of SEQ ID NO:23 and CDR2L region of SEQ ID NO:24; or
iii) CDR1L region of SEQ ID NO:25 and CDR2L region of SEQ ID NO:26.

In particularly preferred embodiments, the multispecific (e.g. bispecific) antibody comprises an anti-BCMA antibody, or antigen binding fragment thereof, comprising a VH region comprising a CDR1H region of SEQ ID NO:21, a CDR2H region of SEQ ID NO:22 and a CDR3H region of SEQ ID NO:17 and a VL region comprising a CDR1L region of SEQ ID NO:27, a CDR2L region of SEQ ID NO:28 and a CDR3L region of SEQ ID NO:20.

In some embodiments, the multispecific (e.g. bispecific) antibody comprises an anti-BCMA antibody, or antigen binding fragment thereof, comprising a VH and a VL selected from the group consisting of:
a) a VH region of SEQ ID NO: 10 and a VL region of SEQ ID NO: 12,
b) a VH region of SEQ ID NO:10 and a VL region of SEQ ID NO:13,
c) a VH region of SEQ ID NO: 10 and a VL region of SEQ ID NO: 14,
d) a VH region of SEQ ID NO:38 and a VL region of SEQ ID NO: 12,
e) a VH region of SEQ ID NO:39 and a VL region of SEQ ID NO: 12,
f) a VH region of SEQ ID NO:40 and a VL region of SEQ ID NO: 12, or
g) a VH region of SEQ ID NO:9 and a VL region of SEQ ID NO: 11.

In particularly preferred embodiments, the anti-BCMA antibody, or antigen binding fragment thereof, comprises a VH region of SEQ ID NO: 10 and a VL region of SEQ ID NO: 14.

In some embodiments, the multispecific (e.g. bispecific) antibody comprises an anti-CD3 antibody, or antigen binding fragment thereof.

Examples of anti-CD3 antibodies include OKT3, TR66, APA 1/1, SP34, CH2527, WT31, 7D6, UCHT-1, Leu-4, BC-3, H2C, HuM291 (visilizumab), Hu291 (PDL), ChAglyCD3 (Otelixizumab), hOKT3γ1 (Ala-Ala) (Teplizumab) and NI-0401 (Foralumab).

The first anti-CD3 antibody generated was OKT3 (muromonab-CD3), a murine antibody binding to the CD3E domain. Subsequent anti-CD3 antibodies include humanized or human antibodies, and engineered antibodies, for example antibodies comprising modified Fc regions.

Anti-CD3 antibodies may recognise an epitope on a single polypeptide chain, for example APA 1/1 or SP34 (Yang S J, The Journal of Immunology (1986) 137; 1097-1100), or a conformational epitope located on two or more subunits of CD3, for example WT31, 7D6, UCHT-1 (see WO2000041474) and Leu-4. Clinical trials have been carried out using several anti-CD3 antibodies, including BC-3 (Anasetti et al., Transplantation 54: 844 (1992) and H2C (WO2008119567A2). Anti-CD3 antibodies in clinical development include HuM291 (visilizumab) (Norman et al., Transplantation. 2000 Dec. 27; 70(12):1707-12.) Hu291 (PDL), ChAglyCD3 (Otelixizumab) (H Waldmann), hOKT3γ1 (Ala-Ala) (Teplizumab) (J Bluestone and Johnson and Johnson) and (NI-0401) Foralumab.

Any anti-CD3 antibody or antigen-binding fragment thereof may be suitable for use in the multispecific (e.g. bispecific) antibodies of the present invention. For example, the multispecific (e.g. bispecific) antibodies may comprise an anti-CD3 antibody selected from OKT3, TR66, APA 1/1, SP34, CH2527, WT31, 7D6, UCHT-1, Leu-4, BC-3, H2C, HuM291 (visilizumab), Hu291 (PDL), ChAglyCD3 (Otelixizumab), hOKT3γ1 (Ala-Ala) (Teplizumab) and NI-0401 (Foralumab). In some embodiments, the multispecific (e.g. bispecific) antibody of the invention comprises a humanized SP34 antibody or antigen-binding fragment thereof.

In some preferred embodiments, the anti-CD3 antibody, or antigen binding fragment thereof, may be derived from SP34 and may have similar sequences and the same properties with regard to epitope binding as antibody SP34.

In some embodiments, the multispecific (e.g. bispecific) antibody comprises an anti-CD3 antibody, or antigen binding fragment thereof, comprising a variable domain VH comprising the heavy chain CDRs of SEQ ID NO: 1, 2 and 3 as respectively heavy chain CDR1H, CDR2H and CDR3H and a variable domain VL comprising the light chain CDRs of SEQ ID NO: 4, 5 and 6 as respectively light chain CDR1L, CDR2L and CDR3L. In some embodiments, the multispecific (e.g. bispecific) antibody comprises an anti-CD3 antibody, or antigen binding fragment thereof, comprising the variable domains of SEQ ID NO:7 (VH) and SEQ ID NO:8 (VL).

In some embodiments, the multispecific (e.g. bispecific) antibody comprises an anti-BCMA antibody, or antigen binding fragment thereof, comprising a CDR3H region of SEQ ID NO: 17 and a CDR3L region of SEQ ID NO:20 and a CDR1H, CDR2H, CDR1L, and CDR2L region combination selected from the group of:
a) CDR1H region of SEQ ID NO:21 and CDR2H region of SEQ ID NO:22, CDR1L region of SEQ ID NO:23, and CDR2L region of SEQ ID NO:24,
b) CDR1H region of SEQ ID NO:21 and CDR2H region of SEQ ID NO:22, CDR1L region of SEQ ID NO:25, and CDR2L region of SEQ ID NO:26,
c) CDR1H region of SEQ ID NO:21 and CDR2H region of SEQ ID NO:22, CDR1L region of SEQ ID NO:27, and CDR2L region of SEQ ID NO:28,
d) CDR1H region of SEQ ID NO:29 and CDR2H region of SEQ ID NO:30, CDR1L region of SEQ ID NO:31, and CDR2L region of SEQ ID NO:32,
e) CDR1H region of SEQ ID NO:34 and CDR2H region of SEQ ID NO:35, CDR1L region of SEQ ID NO:31, and CDR2L region of SEQ ID NO:32,
f) CDR1H region of SEQ ID NO:36 and CDR2H region of SEQ ID NO:37, CDR1L region of SEQ ID NO:31, and CDR2L region of SEQ ID NO:32, and
g) CDR1H region of SEQ ID NO: 15 and CDR2H region of SEQ ID NO: 16, CDR1L region of SEQ ID NO:18, and CDR2L region of SEQ ID NO:19, and
an anti-CD3 antibody, or antigen binding fragment thereof, comprising a CDR1H region of SEQ ID NO: 1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In particularly preferred embodiments, the multispecific (e.g. bispecific) antibody comprises:
an anti-BCMA antibody, or antigen binding fragment thereof, comprising a VH region comprising a CDR1H region of SEQ ID NO:21, a CDR2H region of SEQ ID NO:22 and a CDR3H region of SEQ ID NO: 17 and a VL region comprising a CDR1L region of SEQ ID NO:27, a CDR2L region of SEQ ID NO:28 and a CDR3L region of SEQ ID NO:20; and
an anti-CD3 antibody, or antigen binding fragment thereof, comprising a CDR1H region of SEQ ID NO: 1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In some embodiments, the multispecific (e.g. bispecific) antibody comprises an anti-BCMA antibody, or antigen binding fragment thereof, comprising a VH and a VL selected from the group consisting of:
a) a VH region of SEQ ID NO: 10 and a VL region of SEQ ID NO: 12,
b) a VH region of SEQ ID NO:10 and a VL region of SEQ ID NO:13,
c) a VH region of SEQ ID NO: 10 and a VL region of SEQ ID NO: 14,
d) a VH region of SEQ ID NO:38 and a VL region of SEQ ID NO: 12,
e) a VH region of SEQ ID NO:39 and a VL region of SEQ ID NO: 12,
f) a VH region of SEQ ID NO:40 and a VL region of SEQ ID NO: 12, and

an anti-CD3 antibody, or antigen binding fragment thereof, comprising a VH region of SEQ ID NO:7 and a VL region of SEQ ID NO:8.

In particularly preferred embodiments, the multispecific (e.g. bispecific) antibody comprises an anti-BCMA antibody, or antigen binding fragment thereof, comprising a VH region of SEQ ID NO: 10 and a VL region of SEQ ID NO: 14, and an anti-CD3 antibody, or antigen binding fragment thereof, comprising a VH region of SEQ ID NO:7 and a VL region of SEQ ID NO:8.

Fc

The multispecific, e.g. bispecific, antibodies of the invention may have an Fe or may not have an Fc. In preferred embodiments, the multispecific antibodies of the invention comprise an Fe, preferably a human Fe.

In certain embodiments, the Fe is a variant Fe, e.g., an Fe sequence that has been modified (for example by amino acid substitution, deletion and/or insertion) relative to a parent Fe sequence (for example an unmodified Fe polypeptide that is subsequently modified to generate a variant), to provide desirable structural features and/or biological activity,

Accordingly, the multispecific antibodies, e.g. bispecific antibodies, of the invention may comprise an Fe comprising one or more modifications, typically to alter one or more functional properties of the antibody, such as serum half-life, complement fixation, Fe receptor binding, and/or antigen-dependent cellular cytotoxicity. The Fe may be linked to the anti-BCMA and/or anti-CD3 Fab fragments in the antibodies of the invention.

The presence of an Fe has the advantage of extending the elimination half-life of the antibody. The antibodies, e.g. bispecific antibodies, of the invention may have an elimination half-life in mice or cynomolgus monkeys, preferably cynomolgus monkeys, of longer than 12 hours, preferably 3 days or longer. In some embodiments, the antibodies, e.g. bispecific antibodies, of the invention have an elimination half-life of about 1 to 12 days, which allows at least once or twice/week administration.

Reduced Effector Function

Preferably, the bispecific antibodies of the invention comprise an Fe region (e.g. of IgG1 subclass) that comprises modifications to avoid FcR and C1q binding and minimize ADCC/CDC. This provides the advantage that the bispecific antibody mediates its tumour cell killing efficacy purely by the powerful mechanism of effector cell, e.g. T cell, redirection/activation. Therefore, additional mechanisms of action, such as effects on the complement system and on effector cells expressing FcR, are avoided and the risk of side-effects, such as infusion-related reactions, is decreased.

In preferred embodiments, the antibodies, e.g. bispecific antibodies, of the invention comprise an IgG, particularly IgG1, Fe region comprising the modifications L234A, L235A and P329G (numbered according to EU numbering).

Heterodimerization

The multispecific, e.g. bispecific, antibodies of the invention may be heteromultimeric antibodies. Such heteromultimeric antibodies may comprise modifications in regions involved in interactions between antibody chains to promote correct assembly of the antibodies.

For example, the bispecific antibodies of the invention may comprise an Fe having one or more modification(s) in the CH2 and CH3 domain to enforce Fe heterodimerization. Alternatively or in addition, the bispecific antibodies of the invention may comprise modifications in the CH1 and CL region to promote preferential pairing between the heavy chain and light chain of a Fab fragment.

A number of strategies exist for promoting heterodimerization. These strategies may include the introduction of asymmetric complementary modifications into each of two antibody chains, such that both chains are compatible with each other and thus able to form a heterodimer, but each chain is not able to dimerize with itself. Such modifications may encompass insertions, deletions, conservative and non-conservative substitutions and rearrangements.

Heterodimerization may be promoted by the introduction of charged residues to create favourable electrostatic interactions between a first antibody chain and a second antibody chain. For example, one or more positively charged amino acids amino acid may be introduced into a first antibody chain, and one or more negatively charged amino acids may be introduced into a corresponding positions in a second antibody chain

Alternatively or in addition, heterodimerization may be promoted by the introduction of steric hindrance between contacting residues. For example, one or more residues with a bulky side chain may be introduced into a first antibody chain, and a one or more residues able to accommodate the bulky side chain may be introduced into the second antibody chain.

Alternatively or in addition, heterodimerization may be promoted by the introduction of one or more modification(s) to the hydrophilic and hydrophobic residues at the interface between chains, in order make heterodimer formation more entropically and enthalpically favourable than homodimer formation.

A further strategy for promoting heterodimerization is to rearrange portions of the antibody chains such that each chain remains compatible only with a chain comprising corresponding rearrangements. For example, CrossMAb technology is based on the crossover of antibody domains in order to enable correct chain association. There are three main CrossMAb formats, these are: (i) CrossMAb^{Fab} in which the VH and VL are exchanged and the CH1 and CL are exchanged; (ii) CrossMAb^{VH-VL} in which the VH and VL are exchanged; and (iii) CrossMAb^{CH1-CL} in which the CH1 and CL are exchanged (Klein et al., 2016. MABS, 8(6):1010-1020).

In some embodiments, the bispecific antibodies of the invention may comprise an exchange of the VH and VL. In some embodiments, the antibodies, e.g. bispecific antibodies, of the invention may comprise an exchange of the CH1 and CL. In some embodiments, the antibodies, e.g. bispecific antibodies, of the invention may comprise an exchange of the VH and VL and an exchange of the CH1 and CL.

In preferred embodiments, the antibodies, e.g. bispecific antibodies, of the invention comprise an exchange of the VH and VL.

Other approaches to promoting heterodimerization include the use of a strand exchange engineered domain (SEED) (Davis et al., 2010. Protein Eng Des Sel, 23 (4); 195-202).

A combination of the above strategies may be used to maximise the efficiency of assembly while minimising the impact on antibody stability.

Fc Heterodimerization

In some embodiments, multispecific antibodies, e.g. bispecific antibodies, of the invention may have a heterodimeric Fc, for example they may comprise one heavy chain originating from an anti-BCMA antibody, and one heavy chain originating from an anti-CD3 antibody.

The antibodies, e.g. bispecific antibodies, of the invention may comprise a heterodimeric Fc which comprises one or more modification(s) which promotes the association of the first CH2 and/or CH3 domain with the second CH2 and/or CH3 domain. In preferred embodiments, the one or more modification(s) promote the association of the first CH3 domain with the second CH3 domain, for example by resulting in asymmetric modifications to the CH3 domain. The one or more modification(s) may comprise modifications selected from amino acid insertions, deletions, conservative and non-conservative substitutions and rearrangements, and combinations thereof.

Typically the first CH3 domain and the second CH3 domain are both engineered in a complementary manner so that each CH3 domain (or the heavy chain comprising it) can no longer homodimerize with itself but is forced to heterodimerize with the complementary engineered other CH3 domain (so that the first and second CH3 domain heterodimerize and no homodimers between the two first or the two second CH3 domains are formed).

The multispecific, e.g. bispecific, antibodies of the invention may comprise an Fc having one or more of “knob-into-holes” modification(s), which are described in detail with several examples in e.g. WO 96/027011, Ridgway, J. B., et al., Protein Eng. 9 (1996) 617-621, Merchant, A. M. et al., Nat. Biotechnol. 16 (1998) 677-68, and WO 98/050431.

In this method, the interaction surfaces of the two CH3 domains are altered to increase the heterodimerization of both Fc chains containing these two CH3 domains. One of the two CH3 domains (of the two Fc chains) can be the “knob”, while the other is the “hole”.

Accordingly, the bispecific antibodies of the invention may comprise two CH3 domains, wherein the first CH3 domain of the first Fc chain and the second CH3 domain of the second Fc chain each meet at an interface which comprises an original interface between the antibody CH3 domains, wherein said interface is altered to promote the formation of the antibody.

In some embodiments:

(i) the CH3 domain of one Fc chain is altered, so that within the original interface of the CH3 domain of the one Fc chain that meets the original interface of the CH3 domain of the other Fc chain, an amino acid residue is replaced with an amino acid residue having a larger side chain volume, thereby generating a protuberance within the interface of the CH3 domain of one Fc chain which is positionable in a cavity within the interface of the CH3 domain of the other Fc chain; and

ii) the CH3 domain of the other Fc chain is altered, so that within the original interface of the CH3 domain of the other Fc chain that meets the original interface of the CH3 domain of the one Fc chain, an amino acid residue is replaced with an amino acid residue having a smaller side chain volume, thereby generating a cavity within the interface of the CH3 domain of the other Fc chain within which a protuberance within the interface of the CH3 domain of the one Fc chain is positionable.

Preferably, said amino acid residue having a larger side chain volume is selected from the group consisting of arginine (R), phenylalanine (F), tyrosine (Y), tryptophan (W).

In some embodiments, the multispecific, e.g. bispecific, antibodies of the invention comprise a first CH3 domain comprising modification(s) at positions T366, L368 and Y407, e.g. T366S, L368A, and Y407V (numbered according to EU numbering).

In some embodiments, the multispecific, e.g. bispecific, antibodies of the invention comprise a second CH3 domain comprising a modification at position T366 (“knob modification”), e.g. T366W (numbered according to EU numbering).

In particularly preferred embodiments, the multispecific, e.g. bispecific, antibodies of the invention comprise a first CH3 domain comprising the modifications T366S, L368A, and Y407V, or conservative substitutions thereof, and a second CH3 domain comprising the modification T366W, or a conservative substitution thereof (numbered according to EU numbering).

In one embodiment, the multispecific, e.g. bispecific, antibodies of the invention comprise a first CH3 domain comprising the modification set forth in Table 12 and a second CH3 domain comprising the modifications set forth in Table 12.

TABLE 12
“Knob-into-holes” modification

| First CH3 domain | | Second CH3 domain | |
|---|---|---|---|
| Kabat | EU Numbering | Kabat | EU Numbering |
| T389S | T366S | T389W | T366W |
| L391A | L368A | | |
| Y438V | Y407V | | |

Other techniques for CH3 modifications to enforce heterodimerization are contemplated as alternatives of the invention and are described e.g. in WO96/27011, WO98/050431, EP1870459, WO2007/110205, WO2007/147901, WO2009/089004, WO2010/129304, WO2011/90754, WO2011/143545, WO2012/058768, WO2013/157954, WO2013/157953, and WO2013/096291.

In some embodiments, the bispecific antibody according to the invention is of IgG2 isotype and the heterodimerization approach described in WO2010/129304 can be used.

Other Fc Modifications

In some embodiments, the bispecific antibodies of the invention may comprise an Fc, wherein both CH3 domains are altered by the introduction of cysteine (C) as the amino acid in the corresponding positions of each CH3 domain such that a disulphide bridge between both CH3 domains can be formed. The cysteines may be introduced at position 349 in one of the CH3 domains and at position 354 in the other CH3 domain (numbered according to EU numbering).

Preferably, the cysteine introduced at position 354 is in the first CH3 domain and the cysteine introduced at position 349 is in the second CH3 domain (numbered according to EU numbering).

The Fc may comprise modifications, such as D356E, L358M, N384S, K392N, V397M, and V422I (numbered according to EU numbering). Preferably, both CH3 domains comprise D356E and L358M (numbered according to EU numbering).

Light and Heavy Chain Heterodimerization

In the multispecific, e.g. bispecific, antibodies of the invention, one or more of the immunoglobulin heavy chains and light chains may comprise one or more modification(s), e.g. amino acid modifications that are capable of promoting preferential pairing of a specific heavy chain with a specific light chain when heavy chains and light chains are co-expressed or co-produced. Such modifications can provide considerably improved production/purification without changing biological properties such as binding to BCMA. In particular, by introduction of one or more modification(s) such as amino acid exchanges, light chain mispairing and the formation of side products in production can be significantly reduced and therefore yield is increased and purification is facilitated.

The amino acid exchanges may be substitutions of charged amino acids with opposite charges (for example in the CH1/CL interface) which reduce light chain mispairing, e.g. Bence-Jones type side products.

In preferred embodiments, the one or more modification(s) assist light and heavy chain heterodimerization are amino acid modifications in the light and heavy chains outside of the CDRs.

The one or more modification(s) may be present in the anti-BCMA antibody or antigen-binding fragment thereof. Alternatively, the one or more modification(s) may be present in the anti-CD3 antibody or antigen-binding fragment thereof. In preferred embodiments, the one or more modification(s) are present in the anti-BCMA antibody or antigen-binding fragment thereof.

In some embodiments, the multispecific, e.g. bispecific, antibodies of the invention comprise an immunoglobulin heavy chain comprising a CH1 domain having amino acid modifications K147E/D and K213E/D (numbered according to EU numbering) and a corresponding immunoglobulin light chain comprising a CL domain having amino acid modifications E123K/R/H and Q124K/R/H (numbered according to Kabat). Preferably, the CH1 domain comprises the amino acid modifications K147E and K213E (numbered according to EU numbering) or conservative substitutions thereof, and the corresponding CL domain comprises the amino acid modifications E123R and Q124K or conservative substitutions thereof (numbered according to Kabat). Such multispecific, e.g. bispecific, antibodies can be produced in high yield and can be easily purified.

In one embodiment, the amino acid modifications described in Table 13 can be in the BCMA antibody or in the CD3 antibody.

In one embodiment, the bispecific antibodies of the invention are bivalent, and comprise one anti-BCMA antibody or antigen-binding fragment thereof and one anti-CD3 antibody or antigen-binding fragment thereof (the “1+1” format), wherein:
(a) the BCMA antibody or antigen-binding fragment thereof (e.g. BCMA Fab) comprises a CH1 domain having amino acid modifications set forth in Table 13 and a corresponding CL domain having the amino acid modifications Table 13; or
(b) the CD3 antibody or antigen-binding fragment thereof (e.g. CD3 Fab) comprises a CH1 domain having amino acid modifications set forth in Table 13 and a corresponding CL domain having the amino acid modifications Table 13.

In one embodiment, the bispecific antibodies of the invention are trivalent and comprise two anti-BCMA antibodies or antigen-binding fragments thereof and one anti-CD3 antibody or antigen-binding fragment thereof (the “2+1” format), wherein:
(a) one or both BCMA antibodies or antigen-binding fragments thereof (e.g. BCMA Fabs) comprises a CH1 domain having amino acid modifications set forth in Table 13 and a corresponding CL domain having the amino acid modifications Table 13; or
(b) the CD3 antibody (e.g. CD3 Fab) comprises a CH1 domain having amino acid modifications set forth in Table 13 and a corresponding CL domain having the amino acid modifications Table 13.

In particular, each BCMA antibody (e.g. BCMA Fab) may comprise a CH1 domain having amino acid modifications set forth in Table 13 and a corresponding CL domain having the amino acid modifications Table 13.

TABLE 13
Light and heavy chain heterodimerization modifications

| CH1 domain | | CL domain | |
|---|---|---|---|
| Kabat | EU Numbering | Kabat | EU Numbering |
| K145E | K147E | E123R | E123R |
| K221E | K213E | Q124K | Q124K |

In a preferred embodiment, the multispecific, e.g. bispecific, antibodies of the invention comprise the modifications set forth in Table 13 in combination with the modifications set forth in Table 12. Thus, in one embodiment, the bispecific antibodies of the invention are bivalent, and comprise:
(a) one anti-BCMA antibody or antigen-binding fragment thereof and one anti-CD3 antibody or antigen-binding fragment thereof (the “1+1” format), wherein (i) the BCMA antibody or antigen-binding fragment thereof (e.g. BCMA Fab) comprises a CH1 domain that comprises the amino acid modifications K147E and K213E, and a corresponding CL domain that comprises the amino acid modifications E123R and Q124K (i.e. the modifications set forth in Table 13), or (ii) the CD3 antibody or antigen-binding fragment thereof (e.g. CD3 Fab) comprises a CH1 domain that comprises the amino acid modifications K147E and K213E, and a corresponding CL domain that comprises the amino acid modifications E123R and Q124K (i.e. the modifications set forth in Table 13); and
(b) a first CH3 domain comprising the modifications T366S, L368A, and Y407V, and a second CH3 domain comprising the modification T366W (i.e. the modifications set forth in Table 12).

In one embodiment, the bispecific antibodies of the invention are trivalent and comprise:
(a) two anti-BCMA antibodies or antigen-binding fragments thereof and one anti-CD3 antibody or antigen-binding fragment thereof (the “2+1” format), wherein (i) one or both BCMA antibodies or antigen-binding fragments thereof (e.g. BCMA Fabs) comprises a CH1 domain that comprises the amino acid modifications K147E and K213E, and a corresponding CL domain that comprises the amino acid modifications E123R and Q124K (i.e. the modifications set forth in Table 13), or (ii) the CD3 antibody or antigen-binding fragment thereof (e.g. CD3 Fab) comprises a CH1 domain that comprises the amino acid modifications K147E and K213E, and a corresponding CL domain that comprises the amino acid modifications E123R and Q124K (i.e. the modifications set forth in Table 13); and
(b) a first CH3 domain comprising the modifications T366S, L368A, and Y407V, and a second CH3 domain comprising the modification T366W (i.e. the modifications set forth in Table 12).

In particular, each BCMA antibody (e.g. BCMA Fab) may comprise a CH1 domain having amino acid modifications set forth in Table 13 and a corresponding CL domain having the amino acid modifications Table 13. In preferred embodiments, the first Fc chain is bound at the N-terminus of the Fc to the C-terminus of the first anti-BCMA antibody, and the second Fc chain is bound at the N-terminus of the Fc to the C-terminus of the anti-CD3 antibody.

The multispecific, e.g. bispecific, antibodies of the invention may additionally comprise an amino acid substitution at position 49 of the VL region selected from the group of amino acids tyrosine (Y), glutamic acid (E), serine (S), and histidine (H) and/or an amino acid substitution at position 74 of the VL region that is threonine (T) or alanine (A).

CrossMAb

The multispecific, e.g. bispecific, antibodies of the invention may comprise CrossMAb technology. CrossMAb technology is based on the crossover of antibody domains in order to enable correct chain association. It is used to facilitate multispecific antibody formation. There are three main CrossMAb formats, these are: (i) CrossMAb^{Fab} in which the VH and VL are exchanged and the CH1 and CL are 10 exchanged; (ii) CrossMAb^{VH-VL} in which the VH and VL are exchanged; and (iii) CrossMAb^{CH1-CL} in which the CH1 and CL are exchanged (Klein et al., 2016. MABS, 8(6):1010-1020).

CrossMAb technology is known in the state of the art. Bispecific antibodies wherein the variable domains VL and VH or the constant domains CL and CH1 are replaced by each other are described in WO2009080251 and WO2009080252.

In one or more of the antibodies or antigen-binding fragments within the multispecific, e.g. bispecific, antibodies of the invention, the variable domains VL and VH or the constant domains CL and CH1 may be replaced by each other. In some embodiments, the antibodies, e.g. bispecific antibodies, of the invention may comprise an exchange of the VH and VL and an exchange of the CH1 and CL. Thus, the multispecific, e.g. bispecific, antibodies of the invention may comprise a crossover light chain and a crossover heavy chain. As used herein, a “crossover light chain” is a light chain that may comprise a VH-CL, a VL-CH1 or a VH-CH1. A “crossover heavy chain” as used herein is a heavy chain that may comprise a VL-CH1, a VH-CL or a VL-CL.

In some aspects, there is provided a multispecific, e.g. bispecific, antibody comprising an anti-BCMA antibody of the invention, or an antigen-binding fragment thereof, and an anti-CD3 antibody, or antigen-binding fragment thereof, wherein the multispecific, e.g. bispecific, antibody comprises:
(a) a light chain and a heavy chain of an antibody specifically binding to CD3; and
(b) a light chain and heavy chain of an antibody specifically binding to BCMA,

wherein the variable domains VL and VH and/or the constant domains CL and CH1 are replaced by each other in (i) the anti-BCMA antibody; and/or (ii) the anti-CD3 antibody.

In some embodiments, the variable domains VL and VH or the constant domains CL and CH1 of the anti-CD3 antibody or antigen binding fragment thereof are replaced by each other. More preferably, the variable domains VL and VH of the anti-CD3 antibody or antigen binding fragment thereof are replaced by each other.

In embodiments in which the bispecific antibodies in the 1+1 format have the format: CD3 Fab-BCMA Fab (i.e. when no Fc is present); Fc-CD3 Fab-BCMA Fab; Fc-BCMA Fab-CD3 Fab; or BCMA Fab-Fc-CD3 Fab, the bispecific antibodies may comprise the CrossMAb format, e.g. CrossMAb^{Fab}, CrossMAb^{VH-VL} or CrossMAb^{CH1-CL}. The BCMA Fab may have the CrossMAb format, e.g. CrossMAb^{Fab}, CrossMAb^{VH-VL} or CrossMAb^{CH1-CL}. Alternatively, the CD3 Fab may have the CrossMAb format, e.g. CrossMAb^{Fab}, CrossMAb^{VH-VL} or CrossMAb^{CH1-CL} In preferred embodiments, the CD3 Fab of the bispecific antibody comprises the CrossMAb^{VH-VL} format.

It is especially preferred for the bispecific antibodies of the invention having the 2+1 format to comprise CrossMAb technology. Thus, in embodiments in which the trivalent bispecific antibodies in the 2+1 format have the format: CD3 Fab-BCMA Fab-BCMA Fab; BCMA Fab-CD3 Fab-BCMA Fab (i.e. when no Fc is present); BCMA Fab-Fc-CD3 Fab-BCMA Fab; BCMA Fab-Fc-BCMA Fab-CD3 Fab; or CD3 Fab-Fc-BCMA Fab-BCMA Fab, the bispecific antibodies may comprise 10 the CrossMAb format, e.g. CrossMAb^{Fab}, CrossMAb^{VH-VL} or CrossMAb^{CH1-CL}. The BCMA Fab may have the CrossMAb format, e.g. CrossMAb^{Fab}, CrossMAb^{VH-VL} or CrossMAb^{CH1-CL}. Alternatively, the CD3 Fab may have the CrossMAb format, e.g. CrossMAb^{Fab}, CrossMAb^{VH-VL} or CrossMAb^{CH1-CL}. In preferred embodiments, the CD3 Fab of the bispecific antibody comprises the CrossMAb^{VH-VL} format.

In some embodiments, the bispecific antibodies of the invention having the 1+1 format do not comprise CrossMAb technology, i.e. neither the anti-BCMA antibody nor the anti-CD3 antibody have the variable domains VL and VH or the constant domains CL and CH1 replaced by each other.

### EXEMPLARY EMBODIMENTS

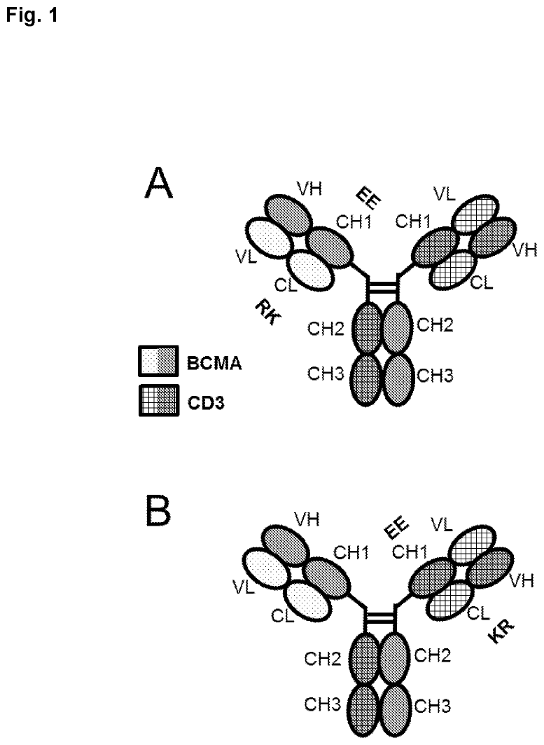

Exemplary embodiments are set out in FIGS. 1-3.

In one embodiment, the bispecific antibodies according to the invention are bivalent bispecific antibodies comprising one Fab fragment of an anti-CD3 antibody, one Fab fragment of an anti-BCMA antibody and one Fc part according to the format BCMA Fab-Fc-CD3 Fab. The anti-BCMA Fab fragment comprises the amino acid modifications set forth in Table 13. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 1A.

In one embodiment, the bispecific antibodies according to the invention are bivalent bispecific antibodies comprising one Fab fragment of an anti-CD3 antibody, one Fab fragment of an anti-BCMA antibody and one Fc part according to the format BCMA Fab-Fc-CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 13. This embodiment is illustrated in FIG. 1B.

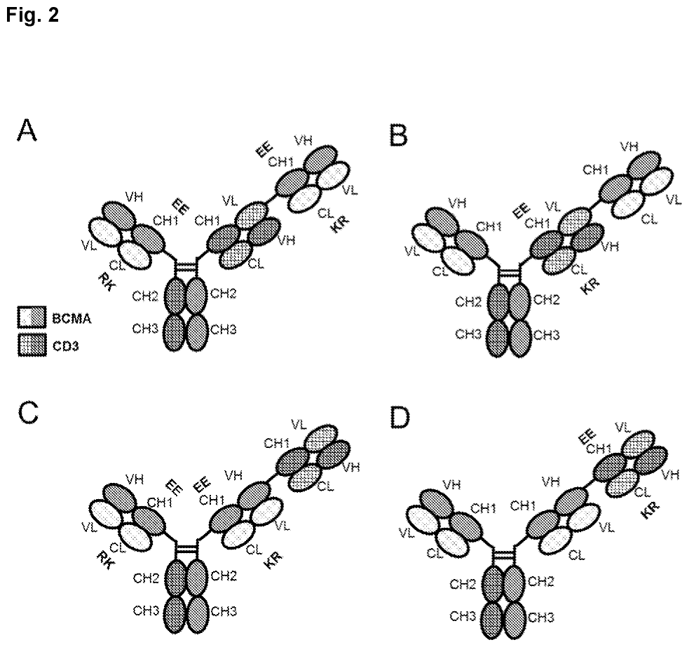

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab. Each anti-BCMA Fab fragment comprises the amino acid modifications set forth in Table 13. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 2A.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 13. This embodiment is illustrated in FIG. 2B.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-BCMA Fab-CD3 Fab. Each anti-BCMA Fab fragment comprises the amino acid modifications set forth in Table 13. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 2C.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-BCMA Fab-CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 13. This embodiment is illustrated in FIG. 2D.

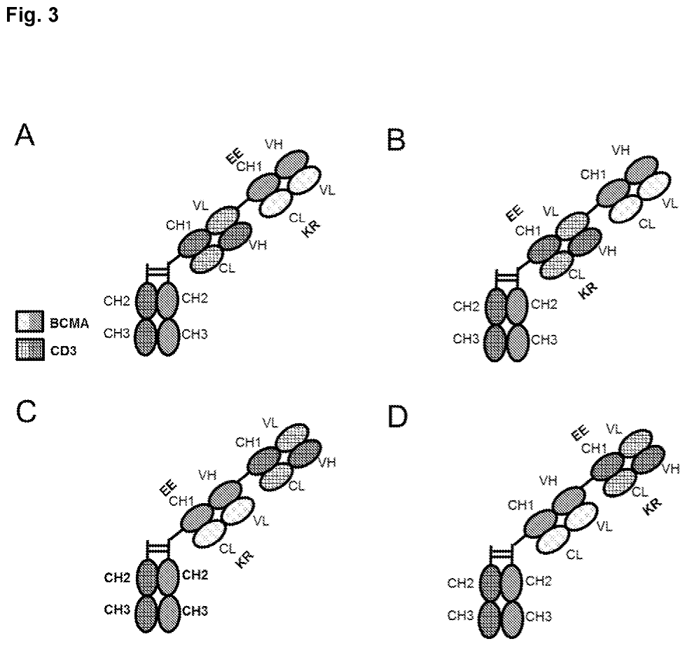

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-CD3 Fab-BCMA Fab. The anti-BCMA Fab fragment comprises the amino acid modifications set forth in Table 13. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 3A.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-CD3 Fab-BCMA Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 13. This embodiment is illustrated in FIG. 3B.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-BCMA Fab-CD3 Fab. The anti-BCMA Fab fragment comprises the amino acid modifications set forth in Table 13. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 3C.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-BCMA Fab-CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 13. This embodiment is illustrated in FIG. 3D.

In one embodiment, the antibodies illustrated in FIG. 2 additionally comprise the modifications set forth in Table 12.

In one aspect, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. Each anti-BCMA Fab fragment comprises a light chain and heavy chain, wherein the heavy chain comprises a CH1 domain which comprises the amino acid modifications K147E and K213E (numbered according to EU numbering) and wherein the light chain comprises a corresponding CL domain which comprises the amino acid modifications E123R and Q124K (numbered according to Kabat) (i.e. the modifications set forth in Table 13). The Fc part comprises a first Fc chain and a second Fc chain, wherein the first Fc chain comprises a first constant domain CH2 and a first constant domain CH3, and the second Fc chain comprises a second constant domain CH2 and a second constant domain CH3. The first Fc chain is bound at the N-terminus of the Fc to the C-terminus of the first anti-BCMA Fab, and the second Fc chain is bound at the N-terminus of the Fc to the C-terminus of the anti-CD3 Fab. The first CH3 domain comprises the modifications T366S, L368A, and Y407V (“hole modifications”) and the second CH3 domain comprises the modification T366W (“knob modification”) (numbered according to EU numbering) (i.e. the modifications set forth in Table 12). Additionally, both Fc chains further comprise the modifications L234A, L235A and P329G, and optionally D356E and L358M (numbered according to EU numbering). Optionally, the first CH3 domain further comprises the amino acid modification S354C, and the second CH3 domain further comprises the amino acid modification Y349C (numbered according to EU numbering) such that a disulphide bridge between both CH3 domains is formed.

In some embodiments, the anti-BCMA Fab fragment comprises a CDR3H region of SEQ ID NO: 17 and a CDR3L region of SEQ ID NO:20 and a CDR1H, CDR2H, CDR1L, and CDR2L region combination selected from the group of
a) CDR1H region of SEQ ID NO:21 and CDR2H region of SEQ ID NO:22, CDR1L region of SEQ ID NO:23, and CDR2L region of SEQ ID NO:24,
b) CDR1H region of SEQ ID NO:21 and CDR2H region of SEQ ID NO:22, CDR1L region of SEQ ID NO:25, and CDR2L region of SEQ ID NO:26,
c) CDR1H region of SEQ ID NO:21 and CDR2H region of SEQ ID NO:22, CDR1L region of SEQ ID NO:27, and CDR2L region of SEQ ID NO:28,
d) CDR1H region of SEQ ID NO:29 and CDR2H region of SEQ ID NO:30, CDR1L region of SEQ ID NO:31, and CDR2L region of SEQ ID NO:32,
e) CDR1H region of SEQ ID NO:34 and CDR2H region of SEQ ID NO:35, CDR1L region of SEQ ID NO:31, and CDR2L region of SEQ ID NO:32,
f) CDR1H region of SEQ ID NO:36 and CDR2H region of SEQ ID NO:37, CDR1L region of SEQ ID NO:31, and CDR2L region of SEQ ID NO:32, and
g) CDR1H region of SEQ ID NO:15 and CDR2H region of SEQ ID NO: 16, CDR1L region of SEQ ID NO:18, and CDR2L region of SEQ ID NO:19, and

the anti-CD3 Fab fragment comprises a CDR1H region of SEQ ID NO: 1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In some embodiments, the anti-BCMA Fab fragment comprises a VH and a VL selected from the group consisting of:
a) a VH region of SEQ ID NO: 10 and a VL region of SEQ ID NO: 12,
b) a VH region of SEQ ID NO:10 and a VL region of SEQ ID NO:13,
c) a VH region of SEQ ID NO: 10 and a VL region of SEQ ID NO: 14,
d) a VH region of SEQ ID NO:38 and a VL region of SEQ ID NO: 12,
e) a VH region of SEQ ID NO:39 and a VL region of SEQ ID NO: 12,
f) a VH region of SEQ ID NO:40 and a VL region of SEQ ID NO: 12, or
g) a VH region of SEQ ID NO:9 and a VL region of SEQ ID NO:11; and

the anti-CD3 Fab fragment comprises a VH region of SEQ ID NO:7 and a VL region of SEQ ID NO:8.

In further embodiments, the bispecific antibody according to the invention comprises the following SEQ ID NOs (as mentioned in Tables 14A and 15B below):
83A10-TCBcv: 45, 46, 47 (×2), 48 (FIG. 2A)
21-TCBcv: 48, 49, 50, 51 (×2) (FIG. 2A)
22-TCBcv: 48, 52, 53, 54 (×2) (FIG. 2A)
42-TCBcv: 48, 55, 56, 57 (×2) (FIG. 2A)

The term “83A10-TCBcv” as used herein refer to a bispecific antibody specifically binding to BCMA and CD3 as specified by its heavy and light chain combination of SEQ ID NO:45, SEQ ID NO:46, SEQ ID NO:47 (2×), and SEQ ID NO:48, and as shown in FIG. 2A and described in EP14179705.

The terms “21-TCBcv, 22-TCBcv, 42-TCBcv” as used herein refer to the respective bispecific antibodies of Mab21, as specified by its heavy and light chain combination of SEQ ID NO:48, SEQ ID NO:49, SEQ ID NO:50, and SEQ ID NO:51 (2×), Mab 22 as specified by its heavy and light chain combinations of SEQ ID NO:48, SEQ ID NO:52, SEQ ID NO:53, and SEQ ID NO:54 (2×), and Mab42 as specified by its heavy and light chain combination of SEQ ID NO:48, SEQ ID NO:55, SEQ ID NO:56, and SEQ ID NO:57-(2×), and as shown in FIG. 2A and described in WO 2017/021450.

In preferred embodiments, the bispecific antibody is 42-TCBcv.

Pharmaceutical Compositions

The multispecific, e.g. bispecific, antibodies of the invention can be administered to the patient as a pharmaceutical composition. Accordingly, the present invention also provides a pharmaceutical composition comprising the multispecific, e.g. bispecific, antibodies of the invention and a pharmaceutically acceptable excipient.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

Examples of suitable excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable excipients include: (1) Dulbecco's phosphate buffered saline, pH.about. 7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

A person skilled in the art would understand that the appropriate choice of excipient or excipients for use with multispecific, e.g. bispecific, antibodies of the invention would depend on the desired properties of the pharmaceutical composition.

Monotherapies and Combinations Therapies

In some embodiments, the treatment comprises the administration of the multispecific, e.g. bispecific, antibody of the invention to the patient as a monotherapy.

In some embodiments, the treatment comprises the administration of the multispecific, e.g. bispecific, antibody of the invention to the patient as a combination therapy, wherein the combination therapy comprises the administration of the multispecific, e.g. bispecific, antibody of the invention and one or more additional therapeutic agents. The term “combination therapy” is meant to encompass administration of the selected therapeutic agents to a single patient, and is intended to include treatments in which the agents are administered by the same or different route of administration or at the same or different time.

In some embodiments, the one or more additional therapeutic agents are selected from the group consisting of thalidomide and an immunotherapeutic derivative thereof, an anti-CD38 antibody, an anti-PD-1 antibody, an anti-PD-L1 antibody, a gamma secretase inhibitor (GSI), an anti-BCMA antibody drug conjugate and anti-BCMA CAR T-cell therapy.

The term “anti-CD38 antibody” as used herein relates to an antibody specifically binding to human CD38. In an embodiment of the invention the anti-CD38 antibody is daratumumab (US20150246123). In an embodiment of the invention the anti-CD38 antibody is isatuximab (SAR650984, U.S. Pat. No. 8,877,899). In an embodiment of the invention the anti-CD38 antibody is MOR202 (WO 2012041800). In an embodiment of the invention the anti-CD38 antibody is Ab79 (U.S. Pat. No. 8,362,211). In an embodiment of the invention the anti-CD38 antibody is Abi9 (U.S. Pat. No. 8,362,211). The dosage of such anti-CD38 antibody is performed according to the state of the art and described in the respective prescribing informations. E.g. Daratumumab dosage is usually 16 mg/kg (www.ema.eoropa.eu).

The term “thalidomide compound” or “thalidomide and an immunotherapeutic derivative” as used herein relates to 2-(2,6-dioxopiperidin-3-yl)-2,3-dihydro-1H-isoindole-1,3-dione and immunotherapeutic derivatives thereof. In an embodiment of the invention the thalidomide compound is selected from the group consisting of, but not limited to, thalidomide (CAS Registry Number 50-35-1), lenalidomide (CAS Registry Number 191732-72-6), pomalidomide (CAS Registry Number 19171-19-8), CC122 (CAS Registry Number 1398053-45-6) and CC-220 (CAS Registry Number 1323403-33-3) and the respective salts (preferably HCl salts 1:1). The chemical formula of CC-122 is 2,6-piperidinedione,3-(5-amino-2-methyl-4-oxo-3(4H-quinazolinyl), hydrochloride (1:1) and of CC-220 it is 2,6-piperidinedione, 3-[1,3-dihydro-4-[[4-(4-morpholinylmethyl)phenyl]methoxy]-1-oxo-2H-isoindol-2-yl]-, (3S)-, hydrochloride (1:1). Methods of preparing CC-220 are described, e.g., in US 20110196150, the entirety of which is incorporated herein by reference.

The dosage of thalidomide compounds is performed according to the state of the art and described in the respective prescribing informations. E.g. Revlimid® (lenalidomide) dosage is usually 25 mg once daily orally on days 1-21 of repeated 28- day cycles (www.revlimid.com) and POMALYST® (pomalidomide) dosage for the treatment of Multiple Myeloma is usually 4 mg per day taken orally on days 1-21 of repeated 28-day cycles (www.celgene.com). In one embodiment, 3-(5-amino-2-methyl-4-oxo-4H-quinazolin-3-yl)-piperidine-2,6-dione is administered in an amount of about 5 to about 50 mg per day.

In one embodiment, CC-122 and CC-220 are administered in an amount of about 5 to about 25 mg per day. In another embodiment, CC-122 and CC-220 are administered in an amount of about 5, 10, 15, 25, 30 or 50 mg per day. In another embodiment, 10 or 25 mg of CC-122 and CC-220 are administered per day. In one embodiment, CC-122 and CC-220 are administered twice per day.

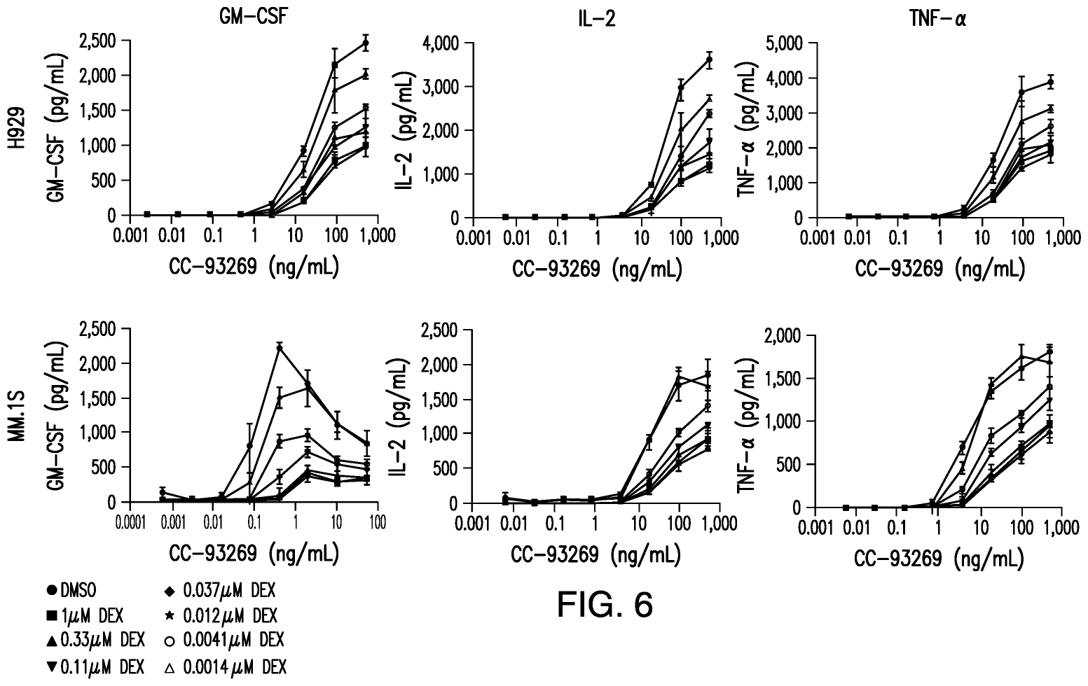

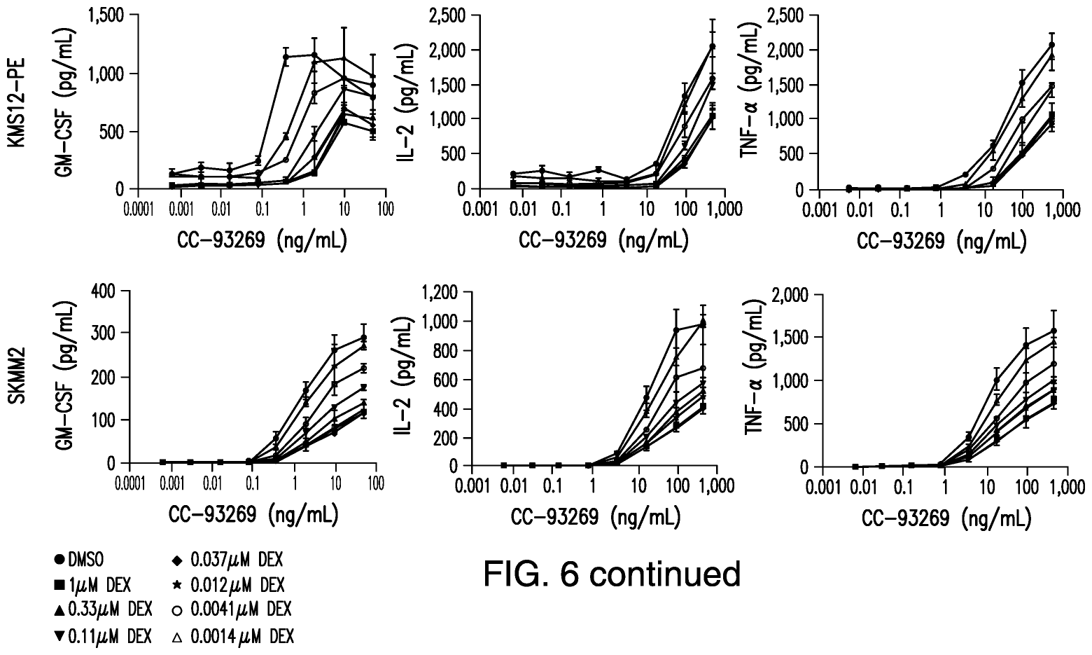

The term “anti-PD-1 antibody” as used herein relates to an antibody specifically binding to human PD-1. Such antibodies are e.g. described in WO2015026634 (MK-3475, pembrolizumab), U.S. Pat. Nos. 7,521,051, 8,008,449, and 8,354,509. Pembrolizumab (Keytruda®, MK-3475) is also described in WO 2009/114335, Poole, R. M. Drugs (2014) 74: 1973; Seiwert, T., et al., J. Clin. Oncol. 32, 5s (suppl; abstr 6011). In an embodiment of the invention the PD-1 antibody is MK-3475 (WHO Drug Information, Vol. 27, No. 2, pages 161-162 (2013)) and which comprises the heavy and light chain amino acid sequences shown in FIG. 6 of WO 2015026634 The amino acid sequence of pembrolizumab is described in WO2008156712 (light chain CDRs SEQ ID NOS:15, 16 and 17 and heavy chain CDRs SEQ ID NOS: 18, 19 and 20)., In an embodiment of the invention the PD-1 antibody is nivolumab (BMS-936558, MDX 1106; WHO Drug Information, Vol. 27, No. 1, pages 68-69 (2013), WO2006/121168 amino acid sequences shown in WO 2015026634). In an embodiment of the invention the PD-1 antibody is; pidilizumab (CT-011, also known as hBAT or hBAT-1; amino acid sequence see WO2003/099196; WO 2009/101611, Fried I. et al.; Neuro Oncol (2014) 16 (suppl 5): v111-v112.). In an embodiment of the invention the PD-1 antibody is MEDI-0680 (AMP-514, WO2010/027423, WO2010/027827, WO2010/027828, Hamid O. et al.; J Clin Oncol 33, 2015 (suppl; abstr TPS3087). In an embodiment of the invention the PD-1 antibody is PDR001 (Naing A. et al.; J Clin Oncol 34, 2016 (suppl; abstr 3060). In an embodiment of the invention the PD-1 antibody is REGN2810 (Papadopoulos K P et al.; J Clin Oncol 34, 2016 (suppl; abstr 3024). In an embodiment of the invention the PD-1 antibody is lambrolizumab (WO2008/156712). In an embodiment of the invention the PD-1 antibody is h409A1 1, h409A16 or h409A17, which are described in WO2008/156712. The dosage of such anti-PD-1 antibody is performed according to the state of the art and described in the respective prescribing informations. E.g. Keytruda® is administered usually in a concentration of 2 mg/kg body weight every three weeks (http://ec.europa.eu/health/documents).

The term “anti-PD-L1 antibody” as used herein relates to an antibody specifically binding to human PD-L1. Such antibodies are e.g. described in WO2015026634, WO2013/019906, WO2010/077634 and U.S. Pat. No. 8,383,796. In an embodiment of the invention the PD-L1 antibody is MPDL3280A (atezolizumab, YW243.55.S70, WO2010/077634, McDermott D F. Et al., JCO Mar. 10, 2016 vol. 34 no. 8 833-842). In an embodiment of the invention the PD-L1 antibody is MDX-1105 (BMS-936559, WO2007/005874, Patrick A. Ott P A et al., DOI: 10.1158/1078-0432, Clinical Cancer Research-13-0143). In an embodiment of the invention the PD-L1 antibody is MEDI4736 (durvalumab, WO 2016/040238 Gilbert J. et al., Journal for ImmunoTherapy of Cancer 20153 (Suppl 2):P152). In an embodiment of the invention the PD-L1 antibody is MSB001071 8C (avelumab, Disis M L. et al., Journal of Clinical Oncology, Vol 33, No 15_suppl (May 20 Supplement), 2015: 5509). In an embodiment of the invention the PD-L1 antibody is the anti-PD-L1 antibody comprising a VH sequence of SEQ ID NO: 16 and a VL sequence of SEQ ID NO: 17 as described in WO2016007235. The dosage of such anti-PD-L1 antibody is performed according to the state of the art and described in the respective prescribing informations. E.g. atezolizumab is administered usually in a concentration of 1200 mg as an intravenous infusion over 60 minutes every 3 weeks (www.accessdata.fda.gov).

The term “gamma secretase” as used herein refers to any protein or protein complex that exhibits gamma secretase activities including binding to a substrate having a gamma secretase cleavage sequence, and catalyzing the cleavage of the gamma secretase cleavage sequence, at a gamma secretase cleavage site, to produce substrate cleavage products. In one embodiment, gamma secretase is a protein complex comprising one or more of the following subunits: presenilin, nicastrin, gamma-secretase subunit APH-1, and gamma-secretase subunit PEN-2.

The term “gamma secretase inhibitor” or “GSI” as used herein refers to any molecule capable of inhibiting or reducing expression and/or function of gamma secretase. In certain embodiment, the GSI reduces expression and/or function of a subunit of gamma secretase (e.g., presenilin, nicastrin, APH-1, or PEN-2). Any form of a “gamma secretase inhibitor” such as a salt, a co-crystal, a crystalline form, a pro-drug, etc., is included within this term. In some embodiments, the GSI is selected from an antibody or antigen-binding fragment, a small molecule, a protein or peptide and a nucleic acid

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In the context of the present invention other examples and variations of the antibodies and methods described herein will be apparent to a person of skill in the art. Other examples and variations are within the scope of the invention, as set out in the appended claims.

All documents cited herein are each entirely incorporated by reference herein, including all data, tables, figures, and text presented in the cited documents.

TABLE 14A
Antibody sequences

| SEQ ID NO: | Name(s) | Amino acid sequences |
|---|---|---|
| 1 | CD3 CDR1H | TYAMN |
| 2 | CD3 CDR2H | RIRSKYNNYATYYADSVKG |
| 3 | CD3 CDR3H | HGNFGNSYVSWFAY |
| 4 | CD3 CDR1L | GSSTGAVTTSNYAN |
| 5 | CD3 CDR2L | GTNKRAP |
| 6 | CD3 CDR3L | ALWYSNLWV |
| 7 | CD3 VH | EVQLLESGGGLVQPGGSLRLSCAASGFTFSTYAMNWVR |
| | | QAPGKGLEWVSRIRSKYNNYATYYADSVKGRFTISRDD |
| | | SKNTLYLQMNSLRAEDTAVYYCVRHGNFGNSYVSWFA |
| | | YWGQGTLVTVSS |
| 8 | CD3 VL | QAVVTQEPSLTVSPGGTVTLTCGSSTGAVTTSNYANWV |
| | | QEKPGQAFRGLIGGTNKRAPGTPARFSGSLLGGKAALT |
| | | LSGAQPEDEAEYYCALWYSNLWVFGGGTKLTVL |
| 9 | 83A10 VH | EVQLLESGGGLVQPGGSLRLSCAASGFTFSSYAMSWVR |
| | | QAPGKGLEWVSAISGSGGSTYYADSVKGRFTISRDNSK |
| | | NTLYLQMNSLRAEDTAVYYCAKVLGWFDYWGQGTLV |
| | | TVSS |
| 10 | Mab21 VH | EVQLLESGGGLVQPGGSLRLSCAASGFTFSDNAMGWV |
| | Mab22 VH | RQAPGKGLEWVSAISGPGSSTYYADSVKGRFTISRDNSK |
| | Mab42 VH | NTLYLQMNSLRAEDTAVYYCAKVLGWFDYWGQGTLV |
| | | TVSS |
| 11 | 83A10 VL | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYGYPPDFTFGQGTKVEIK |
| 12 | Mab21 VL | EIVLTQSPGTLSLSPGERATLSCRASQSVSEYYLAWYQQ |
| | Mab27 VL | KPGQAPRLLIEHASTRATGIPDRFSGSGSGTDFTLTISRLE |
| | Mab33 VL | PEDFAVYYCQQYGYPPDFTFGQGTKVEIK |
| | Mab39 VL | |
| 13 | Mab22 VL | EIVLTQSPGTLSLSPGERATLSCRASQSVSSYYLAWYQQ |
| | | KPGQAPRLLISGAGSRATGIPDRFSGSGSGTDFTLTISRLE |
| | | PEDFAVYYCQQYGYPPDFTFGQGTKVEIK |
| 14 | Mab42 VL | EIVLTQSPGTLSLSPGERATLSCRASQSVSDEYLSWYQQ |
| | | KPGQAPRLLIHSASTRATGIPDRFSGSGSGTDFTLAISRLE |
| | | PEDFAVYYCQQYGYPPDFTFGQGTKVEIK |
| 15 | 83A10 CDR1H | SYAMS |
| 16 | 83A10 CDR2H | AISGSGGSTYYADSVKG |
| 17 | 83A10 CDR3H | VLGWFDY |
| | Mab21 CDR3H | |
| | Mab22 CDR3H | |
| | Mab42 CDR3H | |
| | Mab27 CDR3H | |
| | Mab33 CDR3H | |
| | Mab39 CDR3H | |
| 18 | 83A10 CDR1L | RASQSVSSSYLAW |
| 19 | 83A10 CDR2L | YGASSRAT |
| 20 | 83A10 CDR3L | QQYGYPPDFT |
| | Mab21 CDR3L | |
| | Mab22 CDR3L | |
| | Mab42 CDR3L | |
| 21 | Mab21 CDR1H | DNAMG |
| | Mab22 CDR1H | |
| | Mab42 CDR1H | |
| 22 | Mab21 CDR2H | AISGPGSSTYYADSVKG |
| | Mab22 CDR2H | |
| | Mab42 CDR2H | |
| 23 | Mab21 CDR1L | RASQSVSEYYLAW |
| 24 | Mab21 CDR2L | EHASTRAT |
| 25 | Mab22 CDR1L | RASQSVSSYYLAW |
| 26 | Mab22 CDR2L | SGAGSRAT |
| 27 | Mab42 CDR1L | RASQSVSDEYLSW |
| 28 | Mab42 CDR2L | HSASTRAT |
| 29 | Mab27 CDR1H | SAPMG |
| 30 | Mab27 CDR2H | AISYIGHTYYADSVKG |
| 31 | Mab27 CDR1L | RASQSVSEYYLA |
| | Mab33 CDR1L | |
| | Mab39 CDR1L | |
| 32 | Mab27 CDR2L | HASTRAT |
| | Mab33 CDR2L | |
| | Mab39 CDR2L | |
| 33 | Mab27 CDR3L | QQYGYPPDFT |
| | Mab33 CDR3L | |
| | Mab39 CDR3L | |
| 34 | Mab33 CDR1H | TNAMG |
| 35 | Mab33 CDR2H | AINRFGGSTYYADSVKG |
| 36 | Mab39 CDR1H | QNAMG |
| 37 | Mab39 CDR2H | AISPTGFSTYYADSVKG |
| 38 | Mab27 VH | EVQLLESGGGLVQPGGSLRLSCAASGFTFSSAPMGWVR |
| | | QAPGKGLEWVSAISYIGHTYYADSVKGRFTISRDNSKNT |
| | | LYLQMNSLRAEDTAVYYCAKVLGWFDYWGQGTLVTV |
| | | SS |
| 39 | Mab33 VH | EVQLLESGGGLVQPGGSLRLSCAASGFTFYTNAMGWV |
| | | RQAPGKGLEWVSAINRFGGSTYYADSVKGRFTISRDNS |
| | | KNTLYLQMNSLRAEDTAVYYCAKVLGWFDYWGQGTL |
| | | VTVSS |
| 40 | Mab39 VH | EVQLLESGGGLVQPGGSLRLSCAASGFTFTQNAMGWV |
| | | RQAPGKGLEWVSAISPTGFSTYYADSVKGRFTISRDNSK |
| | | NTLYLQMNSLRAEDTAVYYCAKVLGWFDYWGQGTLV |
| | | TVSS |
| 41 | 83A10 BCMA CH1 | ASTKGPSVFPLAPSSKSTSGGTAALGCLVEDYFPEPVTV |
| | Mab21 BCMA CH1 | SWNSGALTSGVHTFPAVLQSSGLYSLSSVVTVPSSSLGT |
| | Mab22 BCMA CH1 | QTYICNVNHKPSNTKVDEKVEPKSC |
| | Mab42 BCMA CH1 | |
| 42 | 83A10 BCMA CL | RTVAAPSVFIFPPSDRKLKSGTASVVCLLNNFYPREAKV |
| | Mab21 BCMA CL | QWKVDNALQSGNSQESVTEQDSKDSTYSLSSTLTLSKA |
| | Mab22 BCMA CL | DYEKHKVYACEVTHQGLSSPVTKSFNRGEC |
| | Mab42 BCMA CL | |
| 43 | CD3 CH1 | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSC |
| 44 | CD3 CL | ASVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| 45 | 83A10 knob HC | EVQLLESGGGLVQPGGSLRLSCAASGFTFSSYAMSWVR |
| | | QAPGKGLEWVSAISGSGGSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDGGGGSGGG |
| | | GSQAVVTQEPSLTVSPGGTVTLTCGSSTGAVTTSNYAN |
| | | WVQEKPGQAFRGLIGGTNKRAPGTPARFSGSLLGGKAA |
| | | LTLSGAQPEDEAEYYCALWYSNLWVFGGGTKLTVLSS |
| | | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VYTLPPCRDELTKNQVSLWCLVKGFYPSDIAVEWESNG |
| | | QPENNYKTTPPVLDSDGSFFLYSKLTVDKSRWQQGNVF |
| | | SCSVMHEALHNHYTQKSLSLSPGK |
| 46 | 83A10 hole HC | EVQLLESGGGLVQPGGSLRLSCAASGFTFSSYAMSWVR |
| | | QAPGKGLEWVSAISGSGGSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCP |
| | | APEAAGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALGAPIEKTISKAKGQPR |
| | | EPQVCTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 47 | 83A10 LC | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYGYPPDFTFGQGTKVEIKRTVAAPSVFI |
| | | FPPSDRKLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 48 | CD3 LC | EVQLLESGGGLVQPGGSLRLSCAASGFTFSTYAMNWVR |
| | | QAPGKGLEWVSRIRSKYNNYATYYADSVKGRFTISRDD |
| | | SKNTLYLQMNSLRAEDTAVYYCVRHGNFGNSYVSWFA |
| | | YWGQGTLVTVSSASVAAPSVFIFPPSDEQLKSGTASVVC |
| | | LLNNFYPREAKVQWKVDNALQSGNSQESVTEQDSKDS |
| | | TYSLSSTLTLSKADYEKHKVYACEVTHQGLSSPVTKSFN |
| | | RGEC |
| 49 | Mab21 knob HC | EVQLLESGGGLVQPGGSLRLSCAASGFTFSDNAMGWV |
| | | RQAPGKGLEWVSAISGPGSSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDGGGGSGGG |
| | | GSQAVVTQEPSLTVSPGGTVTLTCGSSTGAVTTSNYAN |
| | | WVQEKPGQAFRGLIGGTNKRAPGTPARFSGSLLGGKAA |
| | | LTLSGAQPEDEAEYYCALWYSNLWVFGGGTKLTVLSS |
| | | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VYTLPPCRDELTKNQVSLWCLVKGFYPSDIAVEWESNG |
| | | QPENNYKTTPPVLDSDGSFFLYSKLTVDKSRWQQGNVF |
| | | SCSVMHEALHNHYTQKSLSLSPGK |
| 50 | Mab21 hole HC | EVQLLESGGGLVQPGGSLRLSCAASGFTFSDNAMGWV |
| | | RQAPGKGLEWVSAISGPGSSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCP |
| | | APEAAGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALGAPIEKTISKAKGQPR |
| | | EPQVCTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 51 | Mab21 LC | EIVLTQSPGTLSLSPGERATLSCRASQSVSEYYLAWYQQ |
| | | KPGQAPRLLIEHASTRATGIPDRFSGSGSGTDFTLTISRLE |
| | | PEDFAVYYCQQYGYPPDFTFGQGTKVEIKRTVAAPSVFI |
| | | FPPSDRKLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 52 | Mab22 knob HC | EVQLLESGGGLVQPGGSLRLSCAASGFTFSDNAMGWV |
| | | RQAPGKGLEWVSAISGPGSSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDGGGGSGGG |
| | | GSQAVVTQEPSLTVSPGGTVTLTCGSSTGAVTTSNYAN |
| | | WVQEKPGQAFRGLIGGTNKRAPGTPARFSGSLLGGKAA |
| | | LTLSGAQPEDEAEYYCALWYSNLWVFGGGTKLTVLSS |
| | | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VYTLPPCRDELTKNQVSLWCLVKGFYPSDIAVEWESNG |
| | | QPENNYKTTPPVLDSDGSFFLYSKLTVDKSRWQQGNVF |
| | | SCSVMHEALHNHYTQKSLSLSPGK |
| 53 | Mab22 hole HC | EVQLLESGGGLVQPGGSLRLSCAASGFTFSDNAMGWV |
| | | RQAPGKGLEWVSAISGPGSSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCP |
| | | APEAAGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALGAPIEKTISKAKGQPR |
| | | EPQVCTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 54 | Mab22 LC | EIVLTQSPGTLSLSPGERATLSCRASQSVSSYYLAWYQQ |
| | | KPGQAPRLLISGAGSRATGIPDRFSGSGSGTDFTLTISRLE |
| | | PEDFAVYYCQQYGYPPDFTFGQGTKVEIKRTVAAPSVFI |
| | | FPPSDRKLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 55 | Mab42 knob HC | EVQLLESGGGLVQPGGSLRLSCAASGFTFSDNAMGWV |
| | | RQAPGKGLEWVSAISGPGSSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDGGGGSGGG |
| | | GSQAVVTQEPSLTVSPGGTVTLTCGSSTGAVTTSNYAN |
| | | WVQEKPGQAFRGLIGGTNKRAPGTPARFSGSLLGGKAA |
| | | LTLSGAQPEDEAEYYCALWYSNLWVFGGGTKLTVLSS |
| | | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VYTLPPCRDELTKNQVSLWCLVKGFYPSDIAVEWESNG |
| | | QPENNYKTTPPVLDSDGSFFLYSKLTVDKSRWQQGNVF |
| | | SCSVMHEALHNHYTQKSLSLSPGK |
| 56 | Mab42 hole HC | EVQLLESGGGLVQPGGSLRLSCAASGFTFSDNAMGWV |
| | | RQAPGKGLEWVSAISGPGSSTYYADSVKGRFTISRDNSK |
| | | NTLYLQMNSLRAEDTAVYYCAKVLGWFDYWGQGTLV |
| | | TVSSASTKGPSVFPLAPSSKSTSGGTAALGCLVEDYFPEP |
| | | VTVSWNSGALTSGVHTFPAVLQSSGLYSLSSVVTVPSSS |
| | | LGTQTYICNVNHKPSNTKVDEKVEPKSCDKTHTCPPCP |
| | | APEAAGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALGAPIEKTISKAKGQPR |
| | | EPQVCTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 57 | Mab42 LC | EIVLTQSPGTLSLSPGERATLSCRASQSVSDEYLSWYQQ |
| | | KPGQAPRLLIHSASTRATGIPDRFSGSGSGTDFTLAISRLE |
| | | PEDFAVYYCQQYGYPPDFTFGQGTKVEIKRTVAAPSVFI |
| | | FPPSDRKLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |

Remark: SEQ ID NO: 20 and SEQ ID NO: 33 are identical

TABLE 14B
Antibody sequences (short list)

| | SEQ ID NO: | | | | | | | |
|---|---|---|---|---|---|---|---|---|
| CD3 antibody | VH | VL | CDR1H | CDR2H | CDR3H | CDR1L | CDR2L | CDR3L |
| | SEQ ID NO: | | | | | | | |
| | 7 | 8 | 1 | 2 | 3 | 4 | 5 | 6 |
| BCMA antibody | VH | VL | CDR1H | CDR2H | CDR3H | CDR1L | CDR2L | CDR3L |
| 83A10 | 9 | 11 | 15 | 16 | 17 | 18 | 19 | 20 |
| Mab21 | 10 | 12 | 21 | 22 | 17 | 23 | 24 | 20 |
| Mab22 | 10 | 13 | 21 | 22 | 17 | 25 | 26 | 20 |
| Mab42 | 10 | 14 | 21 | 22 | 17 | 27 | 28 | 20 |
| Mab27 | 38 | 12 | 29 | 30 | 17 | 31 | 32 | 33 |
| Mab33 | 39 | 12 | 34 | 35 | 17 | 31 | 32 | 33 |
| Mab39 | 40 | 12 | 36 | 37 | 17 | 31 | 32 | 33 |

TABLE 15A
Additional constructs

| | SEQ ID NO: | | | |
|---|---|---|---|---|
| Fragment/Construct | 83A10 | Mab21 | Mab22 | Mab42 |
| BCMA CH1 | 41 | 41 | 41 | 41 |
| BCMA CL | 42 | 42 | 42 | 42 |
| CD3 CH1 | 43 | 43 | 43 | 43 |
| CD3 CL | 44 | 44 | 44 | 44 |

TABLE 15B
Additional constructs

| | SEQ ID NO: | | | |
|---|---|---|---|---|
| Construct | 83A10 | Mab21 | Mab22 | Mab42 |
| BCMA VH_CH1cv × CD3 | 45 | 49 | 52 | 55 |
| VL_CH1 Fc knob LALA PG | | | | |
| (knob HC) | | | | |
| BCMAcv HC hole LALA PG | 46 | 50 | 53 | 56 |
| (hole HC) | | | | |
| BCMAcv hum IgG1 LC | 47 | 51 | 54 | 57 |
| (BCMA LC) | | | | |
| CD3 VH_CL (CD3 LC) | 48 | 48 | 48 | 48 |

### EXAMPLES

The examples in this section are offered by way of illustration, and not by way of limitation.

### Example 1: Clinical Study of CC-93269 in Relapsed/Refractory Multiple Myeloma (RRMM): Cohorts 1-7

This study involved 19 RRMM patients who had received ≥3 prior regimens without prior BCMA-directed therapy. Prior treatments included autologous stem cell transplantation, allogenic stem cell transplantation, lenalidomide, pomalidomide, bortezomib, carfilzomib, and daratumumab (DARA). All patients had MM refractory to their last line of therapy.

CC-93269 (42-TCBcv), a bispecific antibody that specifically binds to BCMA bivalently and CD3 monovalently, was administered intravenously over 2 hours on Days 1, 8, 15, and 22 for Cycles 1-3; Days 1 and 15 for Cycles 4-6; and on Day 1 for Cycle 7 and beyond, all in 28-day cycles. 14 patients received a fixed dose of CC-93269 at each of the intervals: 0.15 mg (Cohort 1, n=1), 0.5 mg (Cohort 2, n=1), 1.5 mg (Cohort 3, n=1), 3 mg (Cohort 4, n=4), 6 mg (Cohort 5, n=3) or 10 mg (Cohort 6, n=4). 5 patients received a starting dose of 6 mg on Cycle 1 Day 1, followed by a dose of 10 mg on Cycle 1 Day 8 and thereafter (Cohort 7, n=5).

Efficacy:

Response was assessed using the International Myeloma Working Group (IMWG) Uniform Response Criteria (Rajkumar, 2011a; Kumar, 2016) at every cycle starting on Cycle 2 Day 1 and at the end of treatment. Minimal residual disease (MRD) was assessed by EuroFlow evaluation and NGS (next-generation sequencing) if evaluable. MRD negativity was reported only if a minimum sensitivity of <1 tumour cell in 10⁵ nucleated cells was achieved.

Of the 12 patients treated with ≥6 mg CC-93269 (Cohort 5 to 7), 10 patients achieved a partial response (PR) or better (overall response rate; 83.3%), including 7 (58.3%) with a very good partial response (VGPR) or better and 4 (33.3%) with a stringent complete response (sCR); 9 (75.0%) patients achieved MRD negativity (Table 16). The median time to response was 4.2 weeks (range 4.0-13.1). These data suggested that doses of ≥6 mg CC-93269 are clinically active.

No responses were observed in the 4 patients ofCohort 4 following consecutive doses of 3 mg (Table 16) suggesting that this dose is associated with suboptimal clinical activity.

Pharmacokinetic (PK) data at doses ≥6 mg demonstrated large increases in exposure after multiple doses of CC-93269, suggesting significant target-mediated drug disposition (TMDD) and clearance of BCMA-positive myeloma cells after the first dose. This phenomenon was not observed at 3 mg doses. These data suggests that a 3 mg dose will not be target saturating for the majority of subjects and provides an added margin of safety.

TABLE 16

| | Patients Treated | Patients Treated | |
|---|---|---|---|
| | with <6 mg | with ≥6 mg | All Patients |
| Efficacy, n (%) | (n = 7) | (n = 12) | (N =19) |
| Overall response | 0 (0) | 10 (83.3) | 10 (52.6) |
| (PR or better) | | | |
| VGPR or better | 0 (0) | 7 (58.3) | 7 (36.8) |
| MRD negative | NA | 9 (75.0) | NA |
| Best response | | | |
| Stringent complete | 0 (0) | 4 (33.3) | 4 (21.1) |
| response + complete | | | |
| response (sCR + CR) | | | |
| Very good partial | 0 (0) | 3 (25.0) | 3 (15.8) |
| response (VGPR) | | | |
| Partial response (PR) | 0 (0) | 3 (25.0) | 3 (15.8) |
| Minimal response | 0 (0) | 0 (0) | 0 (0) |
| (MR) | | | |
| Stable disease (SD) | 4 (57.1) | 0 (0) | 4 (21.1) |
| Progressive disease | 2 (28.6) | 1 (8.3) | 3 (15.8) |
| (PD) | | | |
| Not evaluable | 1 (14.3) | 1 (8.3) | 2 (10.5) |

Safety:

CRS prophylaxis was implemented with dexamethasone (up to 20 mg intravenous or equivalent) for the first dose and dose increases in patients receiving ≥6 mg. In Cohorts 1-4, dexamethasone was not used as a premedication.

Patients were monitored for clinical signs and symptoms associated with CRS (Lee, 2014) and CRS events were graded and treated according to the common CTCAE CRS grading scale (Lee, 2015).

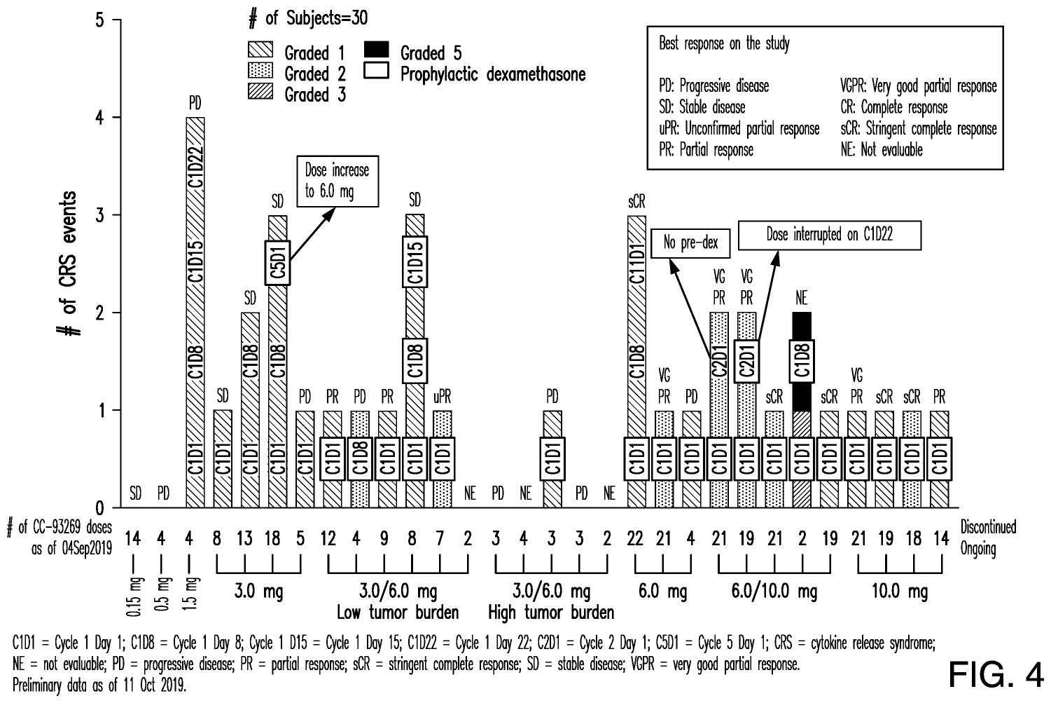

Administration of consecutive doses of ≤3 mg CC-93269 was found to be well-tolerated, despite a CRS prophylactic not being administered at this dose. Cytokine release syndrome with a maximum Grade 1 was observed at a fixed dose of ≤3 mg CC-93269 (FIG. 4).

In Cohort 6, receiving consecutive doses of 6 mg CC-93269, CRS with a maximum Grade 2 was observed, wherein all CRS events occurred in Cycle 1. One patient receiving 6 mg CC-93269 as first dose and 10 mg on Cycle 1 Day 8 (Cohort 7) died on the study in the setting of CRS. In this patient, Grade 3 CRS was observed after the first dose on Cycle 1 Day 1 of 6 mg, and Grade 5 CRS was observed after the second dose on Cycle 1 Day 8 of 10 mg (FIG. 4). It was noted that this patient had extensive extramedullary disease at enrolment, a concurrent documented infection (Clostridium difficile), and a suspected infection at the time of death.

Of 27 CRS events, 8 (29.6%) were managed with dexamethasone and 10 (37.0%) with tocilizumab.

### Example 2: Clinical Study of CC-93269 in Relapsed/Refractory Multiple Myeloma (RRMM): Cohorts 8-9

Cohorts 8 and 9 received a new dosing regimen with the goal of balancing clinical activity and the overall safety profile, particularly minimizing the risk of severe cytokine release syndrome (CRS) in the first cycle.

CC-93269 was administered intravenously over 2 hours on Days 1, 8, 15, and 22 for Cycles 1-3; Days 1 and 15 for Cycles 4-6; and on Day 1 for Cycle 7 and beyond, all in 28-day cycles. Patients received a starting dose of 3 mg on Cycle 1 Day 1, followed by a dose of 6 mg on Cycle 1 Day 8 and thereafter.

Cohorts 8 (n=6) and 9 (n=5) differ in baseline multiple myeloma tumor burden based on percentage of bone marrow plasma cells and/or the presence of extramedullary disease (Cohort 8 including subjects with ≤50% bone marrow plasma cells [biopsy or aspirate] and ≤5 extramedullary lesions and Cohort 9 including subjects with >50% bone marrow plasma cells or >5 extramedullary lesions). Patients with lower (Cohort 8) and higher (Cohort 9) tumour burden were enrolled to evaluate possible differences in tolerability based on the rationale that the only patient of Cohorts 1-7 to experience Grade ≥3 CRS had extensive extramedullary disease at enrolment.

Safety:

In Cohorts 8 and 9, all patients received dexamethasone (up to 20 mg intravenous or equivalent) as prophylaxis for CRS prior to the first dose, the second dose, any dose increase, and after dose interruption of more than 2 weeks occurring during Cycles 1 to 6. Patients were monitored, graded, and treated for CRS in the same way as for Cohorts 1-7 in Example 1.

Of 11 patients receiving the 3/6 mg dose regimen, only 6 (54.5%) experienced CRS compared with 12 of 12 subjects (100%) who received 6 mg, 6/10 mg, or 10 mg doses (Cohorts 5 to 7 of Example 1). In addition to the decreased frequency of CRS events, the severity of CRS in Cohorts 8 and 9 was generally decreased in comparison with Cohorts 5 to 7. All CRS events reported in Cohorts 8 and 9 were Grade 1 (n=6 of 8 [75%]) or Grade 2 (n=2 of 8 [25%]) in severity, and no Grade 3 or higher CRS events were reported. Grade 2 or higher events were reported in only 2 of 11 subjects (18.2%) receiving 3/6 mg compared with 6 of 12 subjects (50%) who received the 6 mg, 6/10 mg, or 10 mg doses (FIG. 4). All CRS events in Cohorts 8 and 9 were resolved with standard treatments, including tocilizumab and/or dexamethasone.

There was no increase observed in CRS frequency or severity in subjects with higher multiple myeloma tumor burden i.e. bone marrow plasma cell percentage or number of extramedullary lesions (Cohort 9) compared with lower tumor burden (Cohort 8).

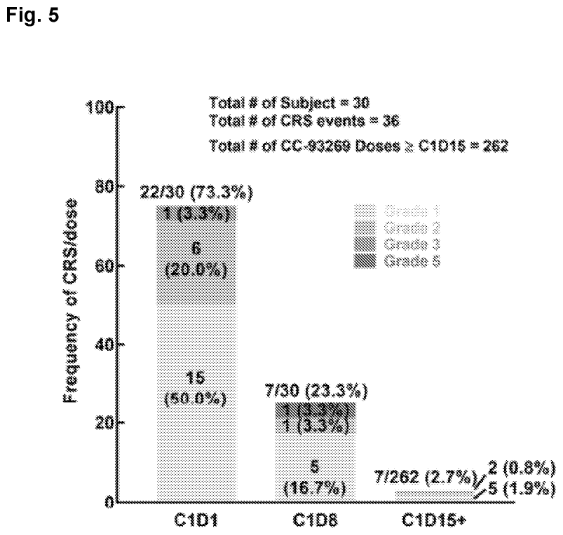

Across all cohorts 1 to 9, of Examples 1 and 2, the median time to first onset of CRS was 1 day, and the maximum time to first onset of CRS was 3 days. The majority of CRS events occurred with the first dose on C1D1 (73.3%), less frequently with the second dose on C1D8 (23.3%) and rarely with subsequent doses (2.7% wherein it was limited to Grade ≤2 events), suggesting that the risk of CRS decreases with continued dosing (FIG. 5).

Across all cohorts 1 to 9, of Examples 1 and 2, the median duration of a CRS event was 2 days (range 1 to 7 days, with only 4 of 36 events lasting more than 3 days).

Efficacy:

Response was assessed in the same manner as with Cohorts 1-7 of Example 1. Subjects of Cohorts 8 and 9 (receiving a maintenance dose of ≥6 mg further to a starting dose of 3 mg) demonstrated improved response compared with subjects of Cohorts 1 to 4 of Example 1 (receiving a maintenance dose of ≤3 mg).

The overall response rates in patients treated with up to 6 mg (3/6 mg and 6 mg) were lower than those treated with up to 10 mg (6/10 mg and 10 mg). Of the patients treated with up to 6 mg (Cohorts 5, 8, and 9), a total of 5 responses have been reported resulting in a preliminary overall response rate of 35.7%. Of the 9 subjects treated with a maintenance dose of 10 mg (Cohorts 6 and 7), a total of 8 responses have been reported resulting in a preliminary overall response rate of 88.9% (Table 17).

These data suggest that a dosing schedule starting with 3/6 mg may decrease CRS, but the higher maintenance dose of 10 mg may improve efficacy, thereby supporting a 3/6/10 mg regimen.

TABLE 17

| | Patients treated | Patients treated | Patients Treated |
|---|---|---|---|
| | with up to 3 mg | with up to 6 mg | with up to 10 mg |
| Efficacy, n (%) | (n = 7) | (n = 14) | (n = 9) |
| Overall response | 0 (0) | 5 (35.7) | 8 (88.9) |
| (PR or better) | | | |
| Best response | | | |
| Stringent complete | 0 (0) | 1 (7.1) | 4 (44.4) |
| response + | | | |
| complete response | | | |
| (sCR + CR) | | | |
| Very good partial | 0 (0) | 1 (7.1) | 3 (33.3) |
| response (VGPR) | | | |
| Partial response | 0 (0) | 3 (21.4) | 1 (11.1) |
| (PR) | | | |
| Minimal response | 0 (0) | 0 (0) | 0 |
| (MR) | | | |
| Stable disease (SD) | 4 (57.1) | 1 (7.1) | 0 |
| Progressive disease | 2 (28.6) | 5 (35.7) | 0 |
| (PD) | | | |
| Not evaluable | 1 (14.3) | 3 (21.4) | 1 (11.1) |

### Example 3: Effect of Dexamethasone on CC-93269-Mediated Cytokine Release, T-Cell Activation, T-Cell Proliferation and Redirected Lysis of MM Cell Lines

Four BCMA-expressing tumour cell lines purchased from ATCC (Manassa, Va.) (Multiple Myeloma cell lines: BCMA high-H-929 (catalog number CRL-9068), BCMA mid-SKMM2 and -MM.1S (catalog number ACC-430) and BCMA low-KMS12-PE (catalog number ACC-606)) were co-cultured with T-cells from 3 independent healthy donors (Bloodworks Northwest, Seattle, Wash.), at a E:T ratio of 1:1, i.e. 1 T cell: 1 myeloma cell. Co-cultures were co-treated with clinically relevant doses of dexamethasone (DEX: 0.0014 μM-1 μM), or control dimethyl sulfoxide (DMSO), and CC-93269 at various concentrations (6.4-500 ng/mL; 0.033-2.6 nM) for 72 hours.

Dexamethasone lowered CC-93269-mediated release of IL-2 (by 48.3-74.1%), GM-CSF (by 47.5-67.8%) and TNF-α (by 46.3-61.8%), as measured using Milliplex human Cytokine/Chemokine magnetic bead multiplex assay (Millipore Sigma, Temecula, Mass.) and according to the manufacturer's recommendations. Percent reduction was determined at the highest concentration of CC-93269 tested comparing co-treatment of 1 μM DEX to the DMSO control. Cytokine levels were calculated according to a standard curve, ranging from 2.29 to 7500 pg/mL, using the ForeCyt software (Intellicyt). The effect of dexamethasone on CC-93269-induced cytokine secretion was determined by calculating percent inhibition of 1 μM dexamethasone compared to the DMSO control and the highest concentration of CC-93269 tested, according to the following formula: Percent inhibition=100×([cytokine concentration, DMSO control]−[cytokine secretion, 1 μM dexamethasone])/[cytokine concentration, DMSO control].

FIG. 6 illustrates cytokine secretion from co-cultures with donor T cells from one healthy donor; results from all 3 independent healthy donors are summarised in Table 18.

TABLE 18

| | % Reduction following 1 μM DEX on CC-93269-induced cytokine secretion | | | | | |
|---|---|---|---|---|---|---|
| Target cell | GM-CSF | | IL-2 | | TNF-α | |
| line | Mean | SD | Mean | SD | Mean | SD |
| H929 | 67.8 | 8.1 | 74.1 | 8.6 | 61.8 | 9.6 |
| MM1S | 66.7 | 6.5 | 57.6 | 8.6 | 50.3 | 4.7 |
| SKMM2 | 56.1 | 5.5 | 56.5 | 3.6 | 46.3 | 9.5 |
| KMS12-PE | 47.5 | 6.2 | 48.3 | 2.2 | 53.9 | 11.8 |

Dexamethasone minimally affected CC-93269-induced CD4 and CD8 T cell activation, proliferation, and redirected lysis of BCMA+ tumour cell lines (e.g. Multiple Myeloma cell lines).

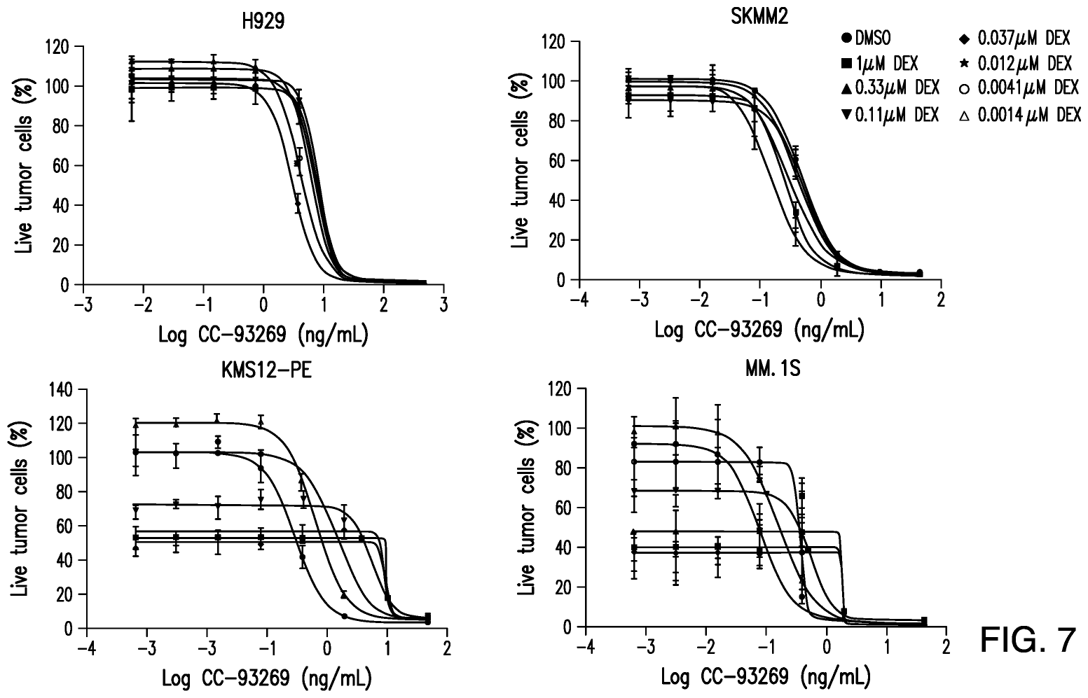

FIG. 7 illustrates CC-93269-induced redirected lysis of BCMA+ tumour cell lines following co-culture with donor T cells from one healthy donor; results from all 3 independent healthy donors are summarised in Table 19. In 96-well round bottom plates and a final volume of 100 μL, 1×10⁴ CellTrace Violet-labeled T-cells were plated with 1×10⁴ CellTrace CFSE-labeled target cells, in the presence of varying concentrations of CC-93269 in combination with varying concentrations of dexamethasone (7-point dose response). The cocultures were incubated at 37° C. with 5% CO₂. After 72 hours of incubation, culture supernatants were harvested and stored at −80° C. The cells were labeled with Live/Dead Fixable Aqua Dead Cell Stain, according to the manufacturer's instructions for 30 minutes at RT. Subsequently, cells were stained with antibodies against the following cell surface markers: CD2, CD3, CD4, CD8, CD69, CD25, HLA-DR, and CD154 in a final volume of 50 μL of flow staining buffer. After staining for cell surface molecules, the cells were washed once with 150 μL flow staining buffer, resuspended in 50 μL of BD Cytofix fixation buffer, and incubated for 30 minutes at RT. Cells were washed with 150 μL of flow staining buffer and resuspended in a final volume of 50 μL of flow staining buffer for analysis on an IQUE Screener plus instrument (Intellicyt, Albuquerque, N. Mex.). One-hundred percent viability was determined by the absolute cell count of live tumour cells at no addition of CC-93269 or dexamethasone; an absolute cell count of 0 live cells was used to define 100% killing of tumour cells. IC50 denotes the concentration of CC-93269 that inhibits a response halfway between the baseline and maximum after a specified exposure time. The IC50 values were calculated using non-linear regression analysis, sigmoidal dose-response using GraphPad Prism (version 5.0) software.

TABLE 19

| | IC50 (ng/ml) | | | | % live tumour cells | | | |
|---|---|---|---|---|---|---|---|---|
| Target | DMSO | | 1 μM Dex | | DMSO | | 1 μM Dex | |
| cell line | Mean | SD | Mean | SD | Mean | SD | Mean | SD |
| H929 | 3.42 | 2.76 | 6.52 | 3.32 | 1.61 | 0.97 | 2.11 | 2.22 |
| MM1S | 0.21 | 0.24 | 3.31 | 3.83 | 1.5 | 0.79 | 0.98 | 0.27 |
| SKMM2 | 0.09 | 0.08 | 0.34 | 0.32 | 2.68 | 0.01 | 3.5 | 0.24 |
| KMS12-PE | 0.2 | 0.13 | 3.61 | 2.48 | 3.71 | 1.09 | 5.8 | 2.66 |

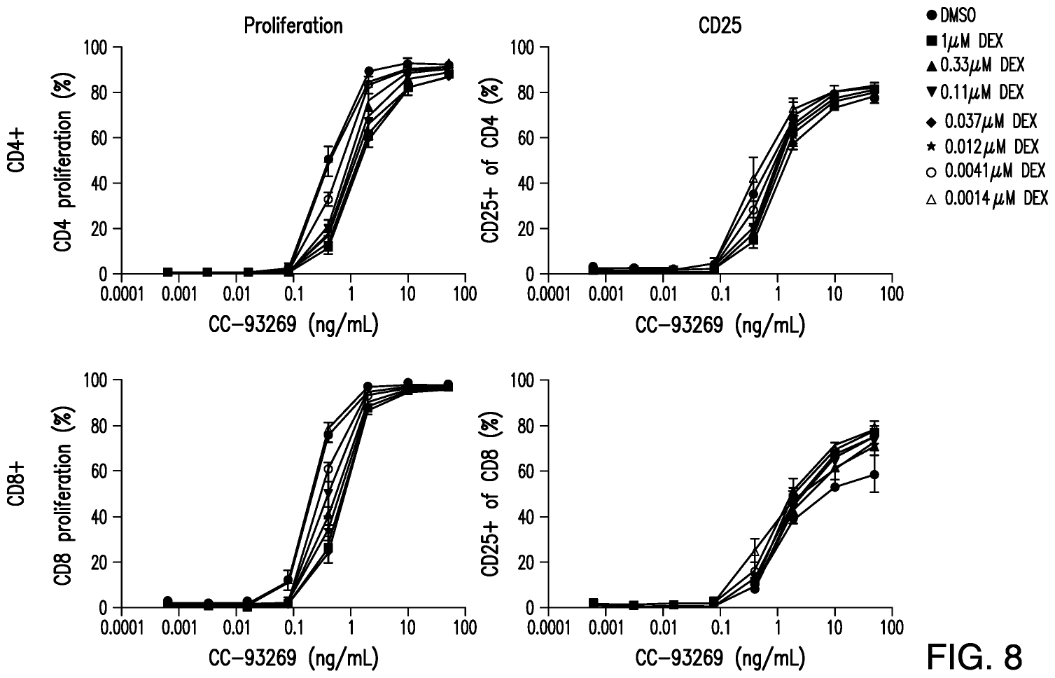

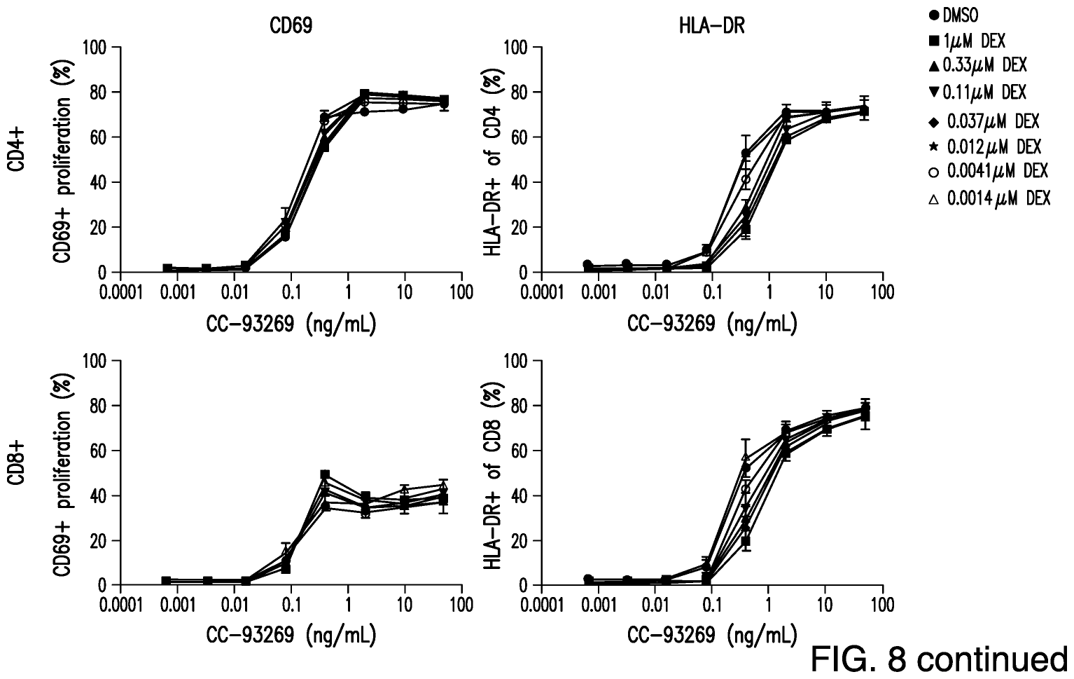

FIG. 8 illustrates CC-93269-induced T cell activation and proliferation following co-culture with donor T cells from one healthy donor. Proliferating CD4+ and CD8+ T-cells in response to increasing amount of CC-93269 was graphed as mean±standard deviation (SD) of triplicate samples of the percentage of CD4+ and CD8+ T-cells that showed dilution of CellTrace Violet following coculture of healthy donor T-cells with tumor cell lines, compared to T-cell only cultures. The percentage of CD4+ and CD8+ T-cells expressing activation markers CD69, CD25, HLA-DR, and CD154 in response to increasing amount of CC-93269 was graphed as mean±SD of triplicate samples of the percentage of CD4+ and CD8+ T-cells expressing the activation markers following coculture of healthy donor T-cells with tumor cell lines, compared to T-cell only cultures. Both the proliferation data and the expression of activation markers was analyzed using ForCyt software (Intellicyt).

Although dexamethasone co-treatment had minimal suppressive impact on CC-93269-mediated cytotoxic activity, it strongly suppressed cytokine secretion, indicating a potential benefit for clinical management of cytokine release syndrome.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

1. A multispecific antibody that binds to BCMA and CD3, for use in treating a disorder associated with BCMA expression in a patient, wherein the treatment comprises the administration of the multispecific antibody in a dosing regimen comprising:
(i) a starting phase, wherein one or more starting doses of the multispecific antibody are administered to the patient; and
(ii) a maintenance phase, wherein a first maintenance dose of the multispecific antibody is administered to the patient, optionally followed by at least one additional maintenance dose of the multispecific antibody,
wherein each maintenance dose is greater than the one or more starting doses.

2. The multispecific antibody for use according to claim 1, wherein the multispecific antibody is a bispecific antibody, optionally wherein the bispecific antibody is a trivalent bispecific antibody comprising two Fab fragments of an anti-BCMA antibody, one Fab fragment of an anti-CD3 antibody, and one Fc portion, wherein the bispecific antibody is in the format BCMA Fab-Fc-CD3 Fab-BCMA Fab.

3. The multispecific antibody for use according to claim 1 or 2, wherein the anti-BCMA antibody, or antigen binding fragment thereof, comprises a VH and a VL selected from the group consisting of:
a) a VH region of SEQ ID NO:10 and a VL region of SEQ ID NO:12,
b) a VH region of SEQ ID NO:10 and a VL region of SEQ ID NO: 13,
c) a VH region of SEQ ID NO:10 and a VL region of SEQ ID NO: 14,
d) a VH region of SEQ ID NO:38 and a VL region of SEQ ID NO: 12,
e) a VH region of SEQ ID NO:39 and a VL region of SEQ ID NO: 12,
f) a VH region of SEQ ID NO:40 and a VL region of SEQ ID NO: 12, or
g) a VH region of SEQ ID NO:9 and a VL region of SEQ ID NO: 11.

4. The multispecific antibody for use according to any one of claims 1-3, wherein the anti-CD3 antibody, or antigen binding fragment thereof, comprises a VH region of SEQ ID NO:7 and a VL region of SEQ ID NO:8.

5. The multispecific antibody for use according to claim 2, wherein the bispecific antibody comprises heavy and light chain polypeptides SEQ ID NO:48, SEQ ID NO:55, SEQ ID NO:56, and SEQ ID NO:57 (×2).

6. The multispecific antibody for use according to any one of claims 1-5, wherein the starting phase comprises a single fixed dose, optionally wherein the single fixed dose is about 1.5 mg to 4.5 mg, e.g. about 3 mg.

7. The multispecific antibody for use according to any one of claims 1-6, wherein the first maintenance dose is a fixed dose of about 4.5 mg to 7.5 mg, e.g. about 6 mg.

8. The multispecific antibody for use according to any one of claims 1-5, wherein the starting phase comprises a single fixed dose, optionally wherein the single fixed dose is about 4.5 mg to 7.5 mg, e.g. about 6 mg.

9. The multispecific antibody for use according to any one of claims 1-6 and 8, wherein the first maintenance dose is a fixed dose of about 8.5 mg to 11.5 mg, e.g. about 10 mg.

10. The multispecific antibody for use according to any one of claims 1-9 wherein the at least one additional maintenance dose is the same as the first maintenance dose.

11. The multispecific antibody for use according to any one of claims 1-9, wherein the at least one additional maintenance dose is greater than the first maintenance dose.

12. The multispecific antibody for use according to any one of claims 1-7, wherein the at least one additional maintenance dose is a fixed dose of about 8.5 mg to 11.5 mg, e.g. about 10 mg.

13. The multispecific antibody for use according to any one of claims 1-12, wherein the patient has developed, or is at risk of developing, an adverse event associated with the administration of the multispecific antibody, and wherein the treatment further comprises the administration of:
a) a steroid, e.g. a corticosteroid;
b) an antagonist of a cytokine receptor or cytokine selected from among GM-CSF, IL-10, IL-10R, IL-6, IL-6 receptor (IL-6R), IFNy, IFNGR, IL-2, IL-2R/CD25, MCP-1, CCR2, CCR4, MIPIβ, CCR5, TNFalpha, TNFR1, IL-1, and IL-1Ralpha/IL-1beta, wherein the antagonist is selected from an antibody or antigen-binding fragment, a small molecule, a protein or peptide and a nucleic acid;
c) a molecule that decreases the regulatory T cell (Treg) population, e.g. cyclophosphamide;
d) an antipyretic, analgesics and/or antibiotics; and/or
e) seizure prophylaxis, e.g. levetiracetam.

14. The multispecific antibody for use according to any one of claims 1-11, wherein the disorder associated with BCMA expression is a BCMA-expressing B-cell cancer, such as multiple myeloma.

15. The multispecific antibody for use according to any one of claims 1-12, wherein the multispecific antibody is administered intravenously.
